(19) United States
(12) Patent Application Publication
(10) Pub. No.: US 2022/0119757 A1
(43) Pub. Date: Apr. 21, 2022

(54) AUTOMATED BIOMANUFACTURING SYSTEMS, FACILITIES, AND PROCESSES

(71) Applicants: Just-Evotec Biologics, Inc., Seattle, WA (US); Merck Sharp & Dohme Corp., Rahway, NJ (US)

(72) Inventors: Michael Wayne Vandiver, Bothell, WA (US); Eva Fan Gefroh, Newcastle, WA (US); Rebecca Eileen McCoy, Seattle, WA (US); Robert James Piper, JR., Issaquah, WA (US); Mark A. Brower, Bound Brook, NJ (US); Nuno J. Dos Santos Pinto, Newark, NJ (US); William N. Napoli, Somerville, NJ (US); Rachel Y. Straughn, Seattle, WA (US); Lisa A. Connell-Crowley, Seattle, WA (US); Megan J. McClure, Seattle, WA (US)

(73) Assignees: Just-Evotec Biologics, Inc., Seattle, WA (US); Merck Sharp & Dohme Corp., Rahway, NJ (US)

(21) Appl. No.: 17/427,873

(22) PCT Filed: Feb. 16, 2020
(86) PCT No.: PCT/US2020/018463

§ 371 Date: Aug. 2, 2021

Related U.S. Application Data
(60) Provisional application No. 62/806,448, filed on Feb. 15, 2019.

(51) Int. Cl. C12M 1/36; C12M 1/00; C12P 21/02

(52) U.S. Cl. CPC C12M 41/48; C12M 23/28; C12P 21/02; C12M 29/04; C12M 47/12; C12M 29/10

(57) ABSTRACT

Disclosed are a process and an automated facility for manufacturing a purified protein of interest. The protein of interest can be a recombinant or naturally occurring protein and/or a therapeutic or other medically useful protein. For example, the disclosed process and automated facility are useful for manufacturing a purified protein drug substance.

This is a U.S. national phase application under 35 U.S.C. § 371 of United States Patent Cooperation Treaty Application No. PCT/US2020/018463, filed Feb. 16, 2020, which claims priority from U.S. Provisional Patent Application Ser. No. 62/806,448, filed in the United States Patent and Trademark Office on Feb. 15, 2019, and which incorporates by reference those PCT and Provisional applications in their entireties.

### BACKGROUND OF THE INVENTION

### 1. Field of the Invention

This invention relates to the field of automated manufacturing facilities and processes for the production of therapeutic proteins.

### 2. Discussion of the Related Art

The biopharmaceutical industry is undergoing major changes, prompted in part by the surge in approvals of new biotherapeutics, higher protein expression rates and increased pressure from the biosimilars market. (Levine et al., Efficient, flexible facilities for the 21st century, BioProcess International 10(11):20-30 (2012)).

An expected surge in the pharmaceutical market share of biologics (from 11% in 2002 to around 20% in 2017), coupled with the need for affordable medicine access in developing regions of the world requires the development of fast, sustainable and cost-effective manufacturing methods. (Walsh, Biopharmaceutical benchmarks 2014, Nature biotechnology 32(10):992-1002 (2014)).

Consequently, there is a need to find biologics manufacturing technology alternatives to traditional batch processing platforms to capitalize on key advantages such as higher throughput, operational flexibility and cost savings, as well as footprint reduction reduced environmental impact. Bioprocessing plants designed to contain a continuous manufacturing process with an integrated upstream and downstream, would allow for rapid facility turnaround, product and capacity flexibility, and lower costs of manufacturing compared to batch culture processing. (See, e.g., Farid et al., Evaluating the economic and operational feasibility of continuous processes for monoclonal antibodies, Continuous Processing in Pharmaceutical Manufacturing pp. 433-456 (2015); Kelley, Industrialization of mAb production technology: the bioprocessing industry at a crossroads, mAbs 1(5):443-452 (2009); Croughan et al., The future of industrial bioprocessing: Batch or continuous?, Biotechnology and Bioengineering 112:648-651 (2015); Pollock et al., Fed-batch and perfusion culture processes: Economic, environmental, and operational feasibility under uncertainty, Biotechnology and Bioengineering 110(1):206-219 (2013)).

The advent of continuous perfusion technologies has supported greater progress in connecting the upstream process equipment in order to operate in a continuous mode. This processing strategy has been valuable for several companies over the past 25 years, helping them to overcome stability problems associated with their products. (Konstantinov et al., White paper on continuous bioprocessing, Journal of Pharmaceutical Sciences 104(3):813-820 (2015)).

Modern cell lines and media have been engineered to target higher cell densities, especially when contrasted with fed-batch processing, with some cultures achieving viable cell densities greater than 100 million cells/mL. (Clincke et al., Very high density of chinese hamster ovary cells in perfusion by alternating tangential flow or tangential flow filtration in wave Bioreactor™—part ii: Applications for antibody production and cryopreservation, Biotechnology Progress 29(3):768-777 (2013)). As a result of this, there has been a shift in the typical biologics manufacturing facility bottleneck from the production bioreactors (upstream processes) to the purification trains (downstream processes), and in particular the chromatography columns due to their dimensional limitations. Purifying large product batch sizes generated by the rising quantities of protein from current production cell lines is not a trivial challenge. (Chon et al., Advances in the production and downstream processing of antibodies, New Biotechnology 28(5):458-463 (2011)). Thus, the problem of integration of upstream biologics manufacturing processes with downstream processes, continues to trouble the biologics manufacturing industry.

Warikoo et al. reported the integration of a continuous capture chromatography step downstream of the production bioreactor, resulting in column size and buffer utilization reductions. (Warikoo et al., Integrated continuous production of recombinant therapeutic proteins, Biotechnology and Bioengineering 109(12):3018-3029. (2012)).

Godawat et al. demonstrated that end-to-end continuous bioprocessing is feasible, but still faces several challenges, including developing robust viral clearance and automation strategies that ensure high product quality. (Godawat et al., End-to-end integrated fully continuous production of recombinant monoclonal antibodies, Journal of Biotechnology 213:13-19 (2015)).

The present invention provides solutions to these challenges and meets the need for automated biologics manufacturing technology alternatives to traditional batch processing platforms.

### SUMMARY OF THE INVENTION

The present invention relates to automated facilities and methods useful in manufacturing a purified protein of interest, such as but not limited to, a therapeutic or other medically useful protein. There are many challenges that are faced in maintaining a perfusion culture of long duration with continuous capture of the protein product. These include the high volume of culture medium that is consumed and the high volume of fluid waste generated from permeate prior to the start of product collection and from the flow through of the capture column during product recovery. With the need to keep a sterile boundary for the waste line it can be prohibitive to collect waste in closed bag systems due to high cost of consumables and labor. There is an increased risk of contamination with long duration perfusion culture and a larger sterile boundary to maintain, including during the continuous capture operation, all in the presence of rich growth medium. Other challenges include maintaining a high viability culture for a long duration and managing discrepant flow rates between connected unit operations, e.g., between a perfusion bioreactor connected to a first chromatography system, connected to a viral inactivation system, connected to a second chromatography system, connected to an optional third chromatography system and/or a viral filtration system, connected to an ultrafiltration/diafiltration system, etc.

The inventive automated facility and process for manufacturing a purified protein of interest (such as but not limited to, a therapeutic or other medically useful protein) meet these and other challenges. In one aspect, the invention encompasses culturing mammalian cells in one or more single-use perfusion bioreactors comprising a liquid culture medium under conditions that allow the cells to secrete the protein into the liquid culture medium for a production cultivation period of at least 10 days, wherein, periodically or continuously, during the production cultivation period, fresh sterile liquid culture medium is added into the one or more perfusion bioreactors, to maintain a constant culture volume in each of the perfusion bioreactor(s), in direct relation to volumes of the culture that are continuously or periodically removed from each of the perfusion bioreactor(s) as volumes of permeate or cell bleed, and wherein the removed volumes of permeate are automatically and fluidly fed from the one or more single-use perfusion bioreactor(s) into a single-use surge vessel and thence into a first chromatography system, whereby the protein is collected in a protein isolate fraction.

In another aspect, the invention encompasses the use of a plurality of different concentrated culture medium component solutions and an aqueous diluent mixed contemporaneously and delivered to the perfusion bioreactor(s), as needed. In another aspect, the invention encompasses closed processing using gamma irradiated or autoclaved ready-to-use disposables, disposable aseptic connectors, tubing welders, and use of chemical cold sterilants on columns. In another aspect, the invention encompasses effective automation and coordinated flow rates between fluidly connected and continuous unit operations, such as viral inactivation and various chromatography systems.

In one embodiment, the present invention relates to an automated facility for manufacturing a purified protein of interest. The purified protein can be a recombinant or naturally occurring protein. The automated facility is controlled by a process automation system (PAS) and includes:

(a) one or more single-use perfusion bioreactors capable of containing a liquid culture medium under conditions that allow cultured cells to secrete the protein into the liquid culture medium for a production cultivation period of at least 10 days; wherein the single-use perfusion bioreactor(s) are adapted to receive fresh sterile liquid culture medium fluidly into each of the perfusion bioreactor(s) in direct relation to volumes of conditioned culture medium that are continuously or periodically removed from each of the perfusion bioreactor(s) as volumes of permeate or cell bleed during the production cultivation period;

(b) a first single-use surge vessel (SUSV1) into which said removed volumes of permeate are automatically and fluidly fed from the one or more single-use perfusion bioreactor(s); and

(c) a first chromatography system, adapted to automatically and fluidly receive cell-free permeate from the SUSV1, whereby the protein is captured in a protein isolate fraction.

The inventive automated facility cam further include:

(d) a low pH or detergent viral inactivation system and, if needed, a neutralization system, adapted to automatically and fluidly receive the protein isolate fraction from the first chromatography system, whereby a virally inactivated product pool comprising the protein is obtained; and

(e) a holding vessel or a second single-use surge vessel, adapted for receiving the virally inactivated product pool.

In some embodiments, the automated facility cam further include:

(f) a second chromatography system adapted to fluidly receive from the holding vessel or the second single-use surge vessel the virally inactivated product pool, whereby a purified product pool comprising the protein is obtained;

(g) an optional third chromatography system and/or a viral filtration system adapted to fluidly receive the purified product pool comprising the protein from the second chromatography system, whereby a virus-free filtrate comprising the protein is obtained; and

(h) an ultrafiltration/diafiltration system adapted to fluidly receive the virus-free filtrate from the second chromatography system or from the third chromatography system and/or the viral filtration system, whereby the purified protein of interest is obtained.

In some embodiments the automated facility for manufacturing a purified protein of interest also includes a plurality of reservoirs, each adapted for containing a concentrated medium component solution or aqueous diluent, and each reservoir being fluidly connected to the perfusion bioreactor(s) directly, or indirectly via an optional mixing vessel, which is adapted for receiving from the plurality of reservoirs the concentrated culture medium component solutions and aqueous diluent at predetermined ratios and contemporaneously mixing them, the optional mixing vessel being fluidly connected directly to the perfusion bioreactor(s).

The invention is also directed to a process for manufacturing a purified protein of interest, which can be a recombinant or naturally occurring protein. The process includes the step of:

(a) culturing mammalian cells in one or more single-use perfusion bioreactors comprising a liquid culture medium under conditions that allow the cells to secrete the protein into the liquid culture medium for a production cultivation period of at least 10 days, wherein, periodically or continuously, during the production cultivation period, fresh sterile liquid culture medium is added into the one or more perfusion bioreactors, being mixed contemporaneously from a plurality of different concentrated medium component solutions and an aqueous diluent, to maintain a constant culture volume in each of the perfusion bioreactor(s), in direct relation to volumes of the culture that are continuously or periodically removed from each of the perfusion bioreactor(s) as volumes of permeate or cell bleed, and wherein the removed volumes of permeate are automatically and fluidly fed from the one or more single-use perfusion bioreactor(s) into a single-use surge vessel and thence into a first chromatography system, whereby the protein is collected in a protein isolate fraction.

The inventive process can further include the step of:

(b) switching the protein isolate fraction into a low pH or detergent viral inactivation system and, if needed, a neutralization system, to obtain a virally inactivated product pool comprising the protein.

In addition, the process can include the further polishing steps of:

(c) introducing the virally inactivated product pool into a second chromatography system to obtain a purified product pool comprising the protein, wherein introducing the virally inactivated product pool into the second chromatography system;

(d) switching the purified product pool comprising the protein into an optional third chromatography system and/or a viral filtration system to obtain a virus-free filtrate comprising the protein; and

(e) switching the virus-free filtrate into an ultrafiltration/diafiltration system to obtain a composition comprising the purified protein of interest.

In a more particular aspect, the present invention relates to an automated facility for manufacturing a purified protein drug substance, i.e., a purified protein of interest for therapeutic or other medical purposes (e.g., prophylactic or diagnostic purposes). The facility includes:

(a) one or more single-use perfusion bioreactors capable of containing a liquid culture medium under conditions that allow cultured mammalian cells to secrete the protein of interest into the medium for a production cultivation period of at least 10 days; wherein the single-use perfusion bioreactor(s) are adapted to receive fresh sterile liquid culture medium fluidly into each of the perfusion bioreactor(s) in direct relation to volumes of conditioned culture medium that are continuously or periodically removed from each of the perfusion bioreactor(s) as volumes of permeate or cell bleed during the production cultivation period, wherein a plurality of reservoirs, each adapted for containing a concentrated medium component solution or aqueous diluent, are fluidly connected to the perfusion bioreactor(s) directly, or indirectly via an optional mixing vessel adapted for receiving from the plurality of reservoirs the concentrated culture medium component solutions and aqueous diluent at predetermined ratios and contemporaneously mixing them, the optional mixing vessel being fluidly connected directly to the perfusion bioreactor(s);

(b) a first single-use surge vessel (SUSV1) into which said removed volumes of permeate (which is free of cells), are automatically and fluidly fed from the one or more single-use perfusion bioreactor(s);

(c) a first chromatography system, adapted to automatically and fluidly receive cell-free permeate from the SUSV1, whereby the protein is captured in a protein isolate fraction;

(d) a low pH or detergent viral inactivation system and, if needed, a neutralization system, adapted to automatically and fluidly receive the protein isolate fraction from the first chromatography system, whereby a virally inactivated product pool comprising the protein is obtained;

(e) a holding vessel or a single-use surge vessel, adapted for receiving the virally inactivated product pool;

(f) a second chromatography system adapted to fluidly receive from the holding vessel or single-use surge vessel the virally inactivated product pool, whereby a purified product pool comprising the protein is obtained;

(g) an optional third chromatography system and/or a viral filtration system adapted to fluidly receive the purified product pool comprising the protein from the second chromatography system, whereby a virus-free filtrate comprising the protein is obtained; and

(h) an ultrafiltration/diafiltration system adapted to fluidly receive the virus-free filtrate from the second chromatography system or from the third chromatography system and/or the viral filtration system, whereby the purified protein drug substance is obtained. Operation of the automated facility is controlled by a process automation system (PAS).

In another more particular aspect, the invention is directed to a process for manufacturing a purified protein drug substance, i.e., a purified protein of interest for therapeutic or other medical purposes (e.g., prophylactic or diagnostic purposes). The purified protein drug substance can be a recombinant or naturally occurring protein. The process involves the steps of:

(a) culturing mammalian cells in one or more single-use perfusion bioreactors comprising a liquid culture medium under conditions that allow the cells to secrete the protein into the medium for a production cultivation period of at least 10 days, wherein, periodically or continuously, during the production cultivation period, fresh sterile liquid culture medium is added into the one or more perfusion bioreactors, to maintain a constant culture volume in each of the perfusion bioreactor(s), in direct relation to volumes of the culture that are continuously or periodically removed from each of the perfusion bioreactor(s) as volumes of permeate or cell bleed, and wherein the removed volumes of permeate are automatically and fluidly fed from the one or more single-use perfusion bioreactor(s) into a single-use surge vessel and thence into a first chromatography system, whereby the protein is collected in a protein isolate fraction;

(b) switching the protein isolate fraction into a low pH or detergent viral inactivation system and, if needed, a neutralization system, to obtain a virally inactivated product pool comprising the protein;

(c) introducing the virally inactivated product pool into a second chromatography system to obtain a purified product pool comprising the protein;

(d) switching the purified product pool comprising the protein into an optional third chromatography system and/or a viral filtration system to obtain a virus-free filtrate comprising the protein; and

(e) switching the virus-free filtrate into an ultrafiltration/diafiltration system to obtain the purified drug substance comprising the protein of interest.

In some embodiments of the a process for manufacturing a purified protein drug substance, the fresh sterile liquid culture medium is mixed contemporaneously from a plurality of different concentrated medium component solutions and an aqueous diluent, before being added into the one or more perfusion bioreactors to maintain a constant culture volume in each of the perfusion bioreactor(s). The foregoing summary is not intended to define every aspect of the invention, and additional aspects are described in other sections, such as the Detailed Description of Embodiments. The entire document is intended to be related as a unified disclosure, and it should be understood that all combinations of features described herein are contemplated, even if the combination of features are not found together in the same sentence, or paragraph, or section of this document.

In addition to the foregoing, the invention includes, as an additional aspect, all embodiments of the invention narrower in scope in any way than the variations defined by specific paragraphs above. For example, certain aspects of the invention that are described as a genus, and it should be understood that every member of a genus is, individually, an aspect of the invention. Also, aspects described as a genus or selecting a member of a genus, should be understood to embrace combinations of two or more members of the genus. Although the applicant(s) invented the full scope of the invention described herein, the applicants do not intend to claim subject matter described in the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE DRAWINGS

FIG. 1A shows a schematic partial process flow diagram of an embodiment of the inventive process showing a plurality of single use reservoirs fluidly connected to a single-use perfusion bioreactor at 500-L scale (bioreactor here designated “500 L SUB”), each reservoir holding a different sterile concentrated culture medium component (the reservoirs shown are designated here with their contents, respectively: “50% glucose”; “Cys/Tyr Stock”; and “600 L Tote 7.5×Conc.”) or aqueous diluent (“1 kL Tote WFI”). “AF”=antifoam, used to minimize foaming in bioreactor; “Base”=sodium carbonate added via automation to maintain bioreactor pH.

FIG. 1B shows a schematic partial process flow diagram of a semi-continuous format embodiment of the inventive process from a 500-L single use bioreactor (“SUB” and “Batch Unit (A)”) to a perfusion system (“Perfusion Skid” and “Batch Unit (A1)”), to a single-use surge vessel (SUSV; “Non-Batch Unit (B1)”, labeled “200 L Portable Mixer”), to a simulated moving bed (SMB) first chromatography system (“Batch Unit (B),” here represented as a single-use, multi-column chromatography system on a cart labeled “SMB Chrom. System”), to elution collection vessels (“Non-Batch Unit (B3)” and “Non-Batch Unit (B4),” each labeled “100L Portable Mixer”) for collecting the protein isolate fraction. In the embodiment shown in FIG. 1B, upstream to the SUSV, an optional filtration system (“Non-Batch Unit (B2),” labeled “Filter Bank”) guards the first chromatography system from particulates; and an optional heat exchanger cools down the permeate material to room temperature (RT), or in some embodiments, to 4° C. or another desired temperature, before introduction to the first chromatography system, depending on the components of chromatography system and stability needs of the protein molecule. In other embodiments (not shown here in FIG. 1B), the protein isolate fraction can be fluidly fed into a second single-use surge vessel (SUSV2), or into at least two automatically switchable alternate single-use collection vessels (SUCV1 and SUCV2), or directly and continuously into viral inactivation system (e.g., a low pH or detergent viral inactivation system). In the schematic of the embodiment shown here in FIG. 1B, a single-use air break assembly (see, also, FIG. 2) is employed to send permeate to waste at the start of perfusion operation (downstream of the SUB and perfusion skid) before the first chromatography system and to drain flow-through waste downstream of the first chromatography system. An additional unit operation between the Perfusion Bank and the Filter Bank (as shown in FIG. 1B) can optionally be included for single-pass tangential flow filtration (SPTFF) to concentrate the perfusion permeate before it flows further downstream toward the first chromatography system.

FIG. 1C shows a schematic partial process flow diagram of an embodiment of the inventive process in which two portable mixers (each shown here as 500-L volume) function as alternating SUCV1 and SUCV2, respectively, operating as part of the viral inactivation system (“viral inactivation skid”), or fluidly feeding thereto, which different embodiments are also represented schematically in FIG. 20A, FIG. 20C, and FIG. 20D. In this FIG. 1C, the SUSVs are shown as 500-L portable mixers large enough to contain the entire pool. Alternatively, in continuous or semi-continuous operation embodiments, any of the SUSVs shown (i.e., SUSV2, SUSV3, or SUSV4) can instead be a different convenient volume (e.g., 50-L, 75-L, or 100-L), or optional. For example, the single-use surge vessel shown before the UF/DF system skid (i.e., SUSV4) can optionally be eliminated in favor of using the UF/DF skid recirculating tank as the surge vessel instead (see, e.g., FIG. 20E). In other embodiments, the single use surge vessel between the second chromatography system and the third chromatography system (i.e., SUSV2) or between the second chromatography system and the viral filtration system (i.e., SUSV2) can be eliminated in favor of running two or more purification or “polishing” steps in tandem (see, e.g., FIG. 20F).

FIG. 1D shows a schematic partial process flow diagram of an embodiment of the inventive process in which two portable mixers (each 500-L volume) function as alternating SUCV1 and SUCV2, respectively, operating as part of the viral inactivation system (“viral inactivation skid”), or fluidly feeding thereto. In the embodiment shown in FIG. 1D, downstream of viral inactivation system (e.g., a low pH viral inactivation system and neutralization system) and depth filtration, the process proceeds in a batch-wise manner with holding vessels between steps or operations (shown as HV1, HV2, HV3, and HV4). However, in other embodiments, any of HV2, HV3, or HV4 can be replaced by surge vessels, or eliminated entirely, in favor of uninterrupted flow between steps or operations, under automated control.

FIG. 2 illustrates schematically an embodiment of a single-use air break assembly. Shown are: (A) connection to waste outlet line of the system, (B) vent filter to introduce air break into flowing liquid, (C) sections of larger and smaller tubing to maintain air break, (D) connection to drain. Tubing drawn with cross-hatching in FIG. 2 (e.g., C) represents braided tubing, but other non-braided tubing of appropriate diameter can be employed instead.

FIG. 3 shows a schematic representation of an embodiment of the SUSV1 (“SUSV”) volume control. The volume limits shown in FIG. 3, upon which a control action is taken, are merely exemplary and can vary based on system component volume and flow rate capacities.

FIG. 4 shows a schematic partial process set-up of chromatography resin and column housing sanitization with a suitable chemical sanitant, e.g., peracetic acid (in FIG. 4 designated, “PAA”) is shown for one embodiment. In this embodiment, a single-use, multi-column continuous chromatography system, for example, a simulated moving bed (SMB) chromatography system is here designated “BioSMB” to represent a Cadence™ BioSMB® PD system (Pall Life Sciences), but other suitable single-use multi-column continuous capture chromatography systems can be employed instead. In this embodiment, the “Aseptic Connector A” can be a AseptiQuik® G connector (Colder Products Company), or the like; “Aseptic Connector B” can be a Kleenpak® Genderless Connector (Pall Biotech), or the like; tubing can be size 73 silicone tubing, or the like; the closed bags for waste can be 10-L single-use bags, or another convenient volume.

FIG. 5 shows schematically various hardware and software components of an embodiment of the inventive automated facility for manufacturing a purified protein of interest, such as a purified protein drug substance that enable communication of data between the different components of the system. However, each of the components shown in the gray boxes in the perfusion system and continuous chromatography system skids, i.e., the Filter Bank, Feed Tank A and Feed Tank B, and Collection Tank A and Collection Tank B are entirely optional as a component of such a skid (batch unit). In other embodiments of the invention, any of these components can be optionally present in a non-batch unit configuration instead, or absent, as desired for the particular manufacturing purpose.

FIG. 6 shows viable cell density for 500-L bioreactor runs and corresponding 2-L comparator satellite bioreactors. The 2-L comparator satellite bioreactors are designated, respectively, “Run 1 R17”, “Run 2 R14,” and “Run 3 R21.”

FIG. 7 shows viability for 500-L bioreactor runs and corresponding 2-L satellite bioreactors.

FIG. 8 shows cell bleed rates for 500-L bioreactor runs and corresponding 2-L satellite bioreactors.

FIG. 9 shows permeate productivity for 500-L bioreactor runs and corresponding 2-L satellite bioreactors.

FIG. 10 shows 500-L single-use bioreactor (SUB) culture volume control using water for injection (WFI) on demand. SUB level (right scale, upper plot) is shown in kilograms; WFI time flow rate (left scale, lower plot and steps) is shown in mL/min. Step changes in WFI flow rate correspond to ramp up in perfusion rate from 0.5 to 1.0, to 2.0 vvd.

FIG. 11 shows representative data comparing osmolality in a 500-L SUB to 2-L satellites (Run 3 shown).

FIG. 12 shows representative data comparing CO₂ in a 500-L SUB to 2-L satellites (Run 3 shown).

FIG. 13 shows representative data comparing base usage in a 500-L SUB to 2-L satellites (Run 3). The 500-L SUB data is shown as the base usage in mL/day normalized to the working volume of the 2-L bioreactors (1.5-L culture volume).

FIG. 14 shows representative data comparing the specific lactate production rate of a 500-L SUB to 2-L satellites (Run 3 shown).

FIG. 15 shows representative data comparing the specific glucose consumption rate of a 500-L single-use perfusion bioreactor to 2-L satellites (“Run 3 R21” and “Run 3 R22”).

FIG. 16A-B shows representative elution profiles of absorbance at 280 nm (A280; Y-axis) profiles of Protein A affinity chromatography for each of three separate Protein A columns (designated in FIG. 16A-B: “Col 1,” “Col 2,” and “Col 3”) on the BioSMB (shown as one elution cycle per day for 17 days; minutes; FIG. 16A), and their respective elution column volumes (CVs; FIG. 16B) for every elution cycle (Run 2 shown). In FIG. 16B, the thick solid plot line represents the Col 1 data (middle plot at Day 17); the thin solid plot line represents the Col 3 data (top plot at Day 17); and the hatched plot line represents the Col 2 data (bottom plot at Day 17).

FIG. 17 shows representative Protein A step yield data (elution yield), shown as the combined daily pool of elution cycles (Run 2 shown). Cumulative elution cycles (“Cumul EL cyc”) are also shown.

FIG. 18 shows representative process related impurities in the combined daily neutralized elution pools (Run 2 shown). HCP=host cell proteins as measured by ELISA assay, DNA=host cell DNA as measured by qPCR assay, LPrA=leached Protein A as measured by ELISA assay.

FIG. 19A-B shows representative SUSV1 culture volume control and a multi-column continuous capture simulated moving bed (SMB) first chromatography system (here designated “BioSMB) load flow rate (Run 2 shown). In FIG. 19A, the load flow rate (right y-axis scale, L/hr) is shown in the upper stepped plot, and the pressure (left y-axis scale, bar) is shown in the jagged lower plot; flow rate was varied ±10% to maintain SUSV control range. In FIG. 19B, the volume (measured as weight, kg) contained in the single-use surge vessel (SUSV) is shown; setpoint was 100 kg, and control range was 70 kg to 130 kg, with an assumed density of 1 kg/L.

FIG. 20A shows a schematic partial process flow diagram of an embodiment of the inventive process in which two alternating single-use collecting vessels (SUCV1 and SUCV2) operate in an alternating manual batch format as the structures where viral inactivation (and neutralization, if needed) is conducted. For example, acidification and neutralization can be conducted alternately in SUCV1 and SUCV2.

FIG. 20B shows a schematic partial process flow diagram of an embodiment of the inventive process in which a single-use surge vessel (shown as SUSV2) is intervening between the first chromatography system, e.g., a simulated moving bed (SMB) chromatography system (designated “BioSMB”) and the viral inactivation/neutralization skid containing the viral inactivation system and neutralization system in an uninterrupted flow or continuous format. In another continuous flow embodiment, SUSV2 can be the vessel in which viral inactivation (and if needed, neutralization) occurs.

FIG. 20C shows a schematic partial process flow diagram of an embodiment of the inventive process in which two alternating single-use collecting vessels (SUCV1 and SUCV2) feed into the viral inactivation/neutralization skid containing the viral inactivation (“VI”) system and neutralization (“Neut”) system in a batch format. The viral inactivation/neutralization systems can be configured with a single tank or two alternating tanks. In the embodiment shown in FIG. 20C, upstream to the SUSV (SUSV1), an optional (“opt”) filter bank guards the first chromatography system from particulates; and an optional heat exchanger (“Heat Exch (opt)”) cools down the permeate material to room temperature (RT), or in some embodiments, to 4° C. or another desired temperature, before introduction to the first chromatography system (designated “BioSMB”), depending on the components of chromatography system and stability needs of the protein molecule.

FIG. 20D shows a schematic partial process flow diagram of an embodiment of the inventive process in which two alternating single-use collecting vessels (SUCV1 and SUCV2) operate as part of, rather than merely feeding into, the viral inactivation/neutralization skid containing the viral inactivation system and neutralization system in an automated batch format. Acidification and neutralization processes are conducted alternately in the tanks of SUCV1 and SUCV2.

FIG. 20E shows a schematic partial process flow diagram of an embodiment of the inventive process in an uninterrupted or continuous flow format. In this embodiment, single-use surge vessels (shown as “Vessel”) are situated between process steps/operations, e.g., upstream to the second chromatography system (shown as “Chrom 2”), the optional third chromatography system (shown as “Chrom 3”), the viral filtration system (shown as “VF”), and, optionally (“*opt”), before the ultrafiltration/diafiltration (“UF/DF”) system, because the recirculating tank of the UF/DF skid can be used as a surge vessel instead. As illustrated in FIG. 20E, optional (“opt”) in-line conditioning of the pH and/or conditioning of the conductivity load of the outflow from each operation before the next operation can be automatically conducted, as needed. In other alternative embodiments, pH conditioning and/or conditioning of the conductivity load of the outflow from each operation can occur in one or more of the “vessels” or SUSVs (i.e., in-vessel conditioning) that are illustrated between the operations in FIG. 20E.

FIG. 20F-G show a schematic partial process flow diagram of an embodiment of the inventive process in an uninterrupted or continuous flow format in which two or more steps/operations are run in tandem without intervening single-use surge vessels, e.g., (in FIG. 20F) upstream to the second chromatography system (shown as “Chrom 2”), the optional third chromatography system (shown as “Chrom 3”), and/or the viral filtration system (shown as “VF”); or, e.g., (in FIG. 20G) the viral filtration system (shown as “VF”), inline depth filtration (shown in FIG. 20G as “Inline DF; also known as ILDF), and single pass tangential flow filtration (shown as “SPTFF”). From single pass tangential flow filtration the flow can be continuous to UF/DF, or can be collected in a holding vessel for batch application of UF/DF. When the inventive process involves switching the virus-free filtrate into an ultrafiltration/diafiltration system to obtain a composition comprising the purified protein of interest, It is preferred that the operating pressure of the SPTFF step is controlled in a range of about 0.25 psi to about 60 psi (or about 0.25 psi to about 45 psi; or about 0.25 psi to about 30 psi; or about 0.25 psi to about 15 psi; or about 0.25 psi to about 5 psi), and/or that the operating pressure of the ILDF step is controlled in a range of about 0.25 psi to about 60 psi (or about 0.25 psi to about 45 psi; or about 0.25 psi to about 30 psi; or about 0.25 psi to about 15 psi; or about 0.25 psi to about 5 psi).

FIG. 21 shows a comparison of high molecular weight species (HMW), as measured by size exclusion high performance liquid chromatography (SE-HPLC) post-Protein A chromatography protein isolate fraction (“PrAEL HMW”) and the low pH viral inactivated and neutralized virally inactivated product pool (“VI/Neut Pool HMW”).

FIG. 22 shows a schematic representation of a continuous embodiment of the inventive process for manufacturing a purified protein of interest, or a purified protein drug substance, from single-use perfusion bioreactor (“SUB”) to final formulation step comprising two-stages of single-pass tangential flow filtration (“SPTFF”) and in-line diafiltration (“ILDF”) modules. The first chromatography system was a Protein A affinity chromatography capture step performed using a Cadence™ BioSMB® PD system (Pall; designated “BioSMB”); a low pH viral inactivation system (“2-Tank VI”) was included in the process; a second chromatography system included ionic exchange chromatography (“IEX”). Single-use surge vessels (“SUSV”) are shown employed between unit operations.

FIG. 23 shows a schematic representation of the depth filter cart of the example illustrated in FIG. 22 and its post-use flush system. The cart in this embodiment was comprised of two filter trains, each with a depth filter (designated here, “DF-1” and “DF-2”) each followed by a sterile filter (designated here, respectively, “SF-1” and “SF-2”). The differential pressure was monitored across each filter with pressure transducers (designated here as “P1,” “P2,” “P3,” and “P4”). At a specified differential pressure limit, the filter train can be switched using automated valves (two triangles pointing to each other with the tips of the inner points touching represent two-way valves). Fouled filters can be replaced and flushed for later re-use while the new filters are in operation.

FIG. 24 shows a detailed schematic representation of the SPTFF and ILDF systems in an exemplary continuous format embodiment, as described in Example 5. The differential pressure was monitored across each filter with pressure transducers (designated here as “P1,” “P2,” “P3,” and “P4”). Optional “Break Tank” indicates an optional surge vessel. At a specified differential pressure limit, the filter train can be switched using automated valves (two triangles pointing to each other with the tips of the inner points touching represent two-way valves; three triangles pointing to each other with the tips of the inner points touching represent three-way valves). Fouled filters can be replaced and flushed for later re-use while the new filters are in operation.

### DETAILED DESCRIPTION OF EMBODIMENTS

The section headings used herein are for organizational purposes only and are not to be construed as limiting the subject matter described.

### Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular. Thus, as used in this specification and the appended claims, the singular forms “a”, “an” and “the” include plural referents unless the context clearly indicates otherwise. For example, reference to “a protein” includes one protein or a plurality of proteins; reference to “a bioreactor” includes one bioreactor or a plurality of bioreactors.

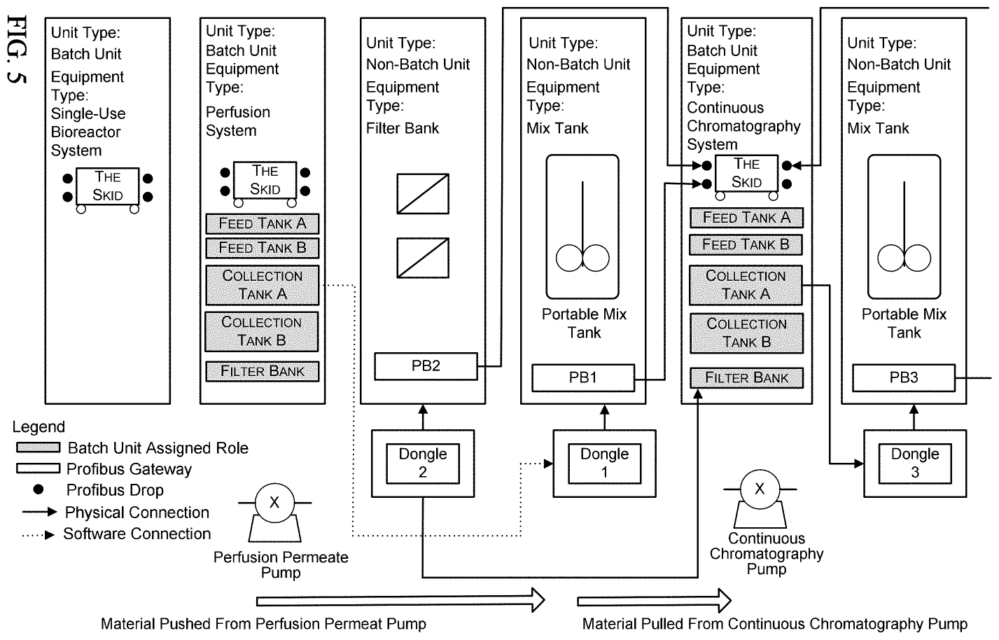

The present invention is directed to an integrated, continuous or semi-continuous, and automated process for manufacturing a purified protein of interest (for example, but not limited to, a purified protein drug substance). The inventive process is performed under aseptic operational conditions and involves automation-controlled regulation of chromatography system flow rates. (See, e.g., FIG. 5).

The various steps of the process can be performed within the automated facility, either in a single cleanroom or a plurality of separate modular cleanrooms, which can, optionally, be automation-controlled.

The term “integrated,” in connection with a process for manufacturing a purified protein of interest (e.g., but not limited to, a protein drug substance), means that one or more upstream steps and/or downstream steps in the manufacturing process are performed under common or coordinated control, based on programmed commands as modified by current sensory feedback of defined parameters in relation to set-points or flow rates. The term “coordinated” means that two or more operations, steps, processes, components, or systems, are controlled, regulated, or scheduled in a relationship that will ensure efficiency or harmony of their functioning toward a single purpose.

“Upstream” processes include, but are not limited to, e.g., culturing the recombinant host cells; removing cells from the permeate; and fluidly feeding volumes of cell-free permeate from the one or more perfusion bioreactor(s) into a single-use surge vessel. “Downstream” process steps include, but are not limited to, e.g., product capture and purification in a first chromatography system; switching the protein isolate fraction into a viral inactivation system, wherein the viral inactivation system is a low pH or detergent viral inactivation system and, if needed (e.g., in low pH viral inactivation system embodiments), a neutralization system; introducing the virally inactivated product pool into a second chromatography system; switching the purified product pool into a third chromatography system and/or a viral filtration system; and/or switching virus-free filtrate into an ultrafiltration/diafiltration system.

“Virally inactivated product pool” includes, protein product-containing material obtained by operation of the viral inactivation system (and if needed the neutralization system). For purpose of the invention, “virally inactivated product pool” encompasses such material obtained by operation of the viral inactivation system (and if needed the neutralization system), and subsequently filtered by (optional) depth filtration to yield a filtered virally inactivated product pool (FVIP), before further downstream processing.

A “continuous” format of a manufacturing process or system means a processing modality wherein a perfusion bioreactor is fluidly connected to a continuous capture chromatography step (e.g., processing by a first chromatography system) in an uninterrupted flow coming from the bioreactor (directly or indirectly via intervening unit operations) to the first chromatography system, which is followed by, and fluidly connected in an uninterrupted flow to, a downstream viral inactivation step, and optionally, in an uninterrupted flow to depth filtration. Further downstream product purification steps (e.g., a second chromatography system, an optional third chromatography system, viral filtration, and processing by ultrafiltration/diafiltration) are fluidly connected, all in an uninterrupted flow to the afore-mentioned upstream processing steps and successively to each other, with optional intervening surge vessels.

A “semi-continuous” format of a manufacturing process means a processing modality wherein a perfusion bioreactor is fluidly connected to a continuous capture chromatography step (e.g., processing by a first chromatography system) in an uninterrupted flow, and to processing by a viral inactivation system, and optionally in an uninterrupted flow to depth filtration, and storage of virally inactivated product pool in a holding vessel (HV1). Temporary storage of the virally inactivated product pool in the holding vessel is subsequently followed by one or more batch downstream processing step(s), which step(s) can be successively fluidly connected to each other in an uninterrupted flow, e.g., a second chromatography system, an optional third chromatography system, and processing by ultrafiltration/diafiltration, with optional intervening surge vessels or holding vessels (i.e., holding vessels if there are two or more batch steps or operations), as the case may be.

A “perfusion bioreactor” is a bioreactor for culturing cells in which equivalent volumes of culture medium can be added and removed from the reactor while the cells are retained in the bioreactor. A perfusion bioreactor includes a bioreactor and an operably attached perfusion system, which provides a steady source of fresh nutrient medium and removal of cell waste products. The bioreactor and the perfusion system of the perfusion bioreactor can be separate mechanical units that operate in coordination. Numerous commercially available examples include, but are not limited to, a variety of Xcellerex® brand single-use bioreactors (SUBs; GE Healthcare Life Sciences) and KrosFlo® brand perfusion flow-path assemblies and systems (Spectrum; Repligen), which bioreactors and perfusion systems can be suitably combined into a perfusion bioreactor by the skilled practitioner. Alternatively, the bioreactor and the perfusion system can be assembled into a single mechanical unit, for example, but not limited to, a 3D Biotek brand perfusion bioreactor (Sigma-Aldrich). Secreted protein products in the bioreactor can be continuously harvested by microfiltration during the process of removing medium via the perfusion system, the protein of interest thus being isolated in a microfilter permeate exiting the perfusion system.

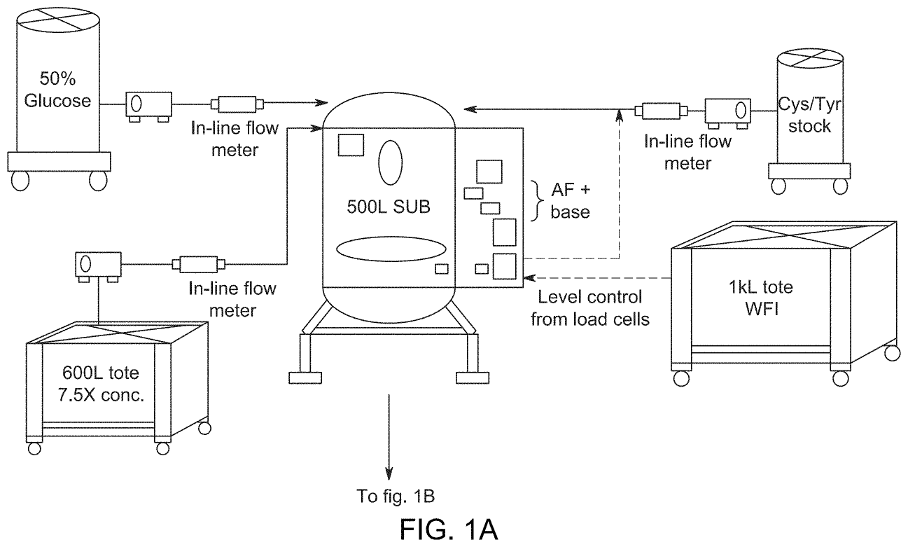

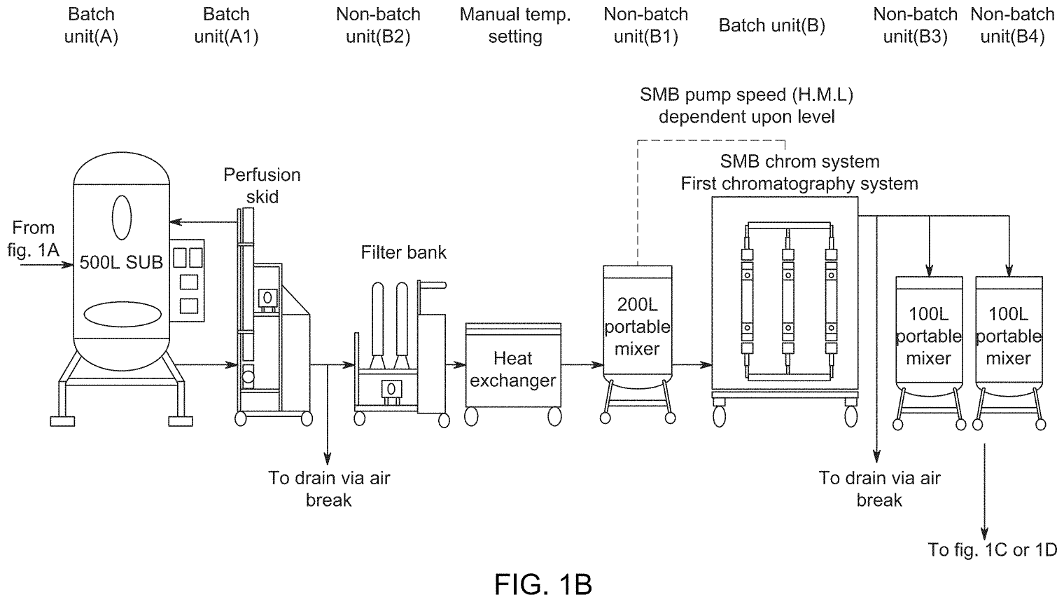

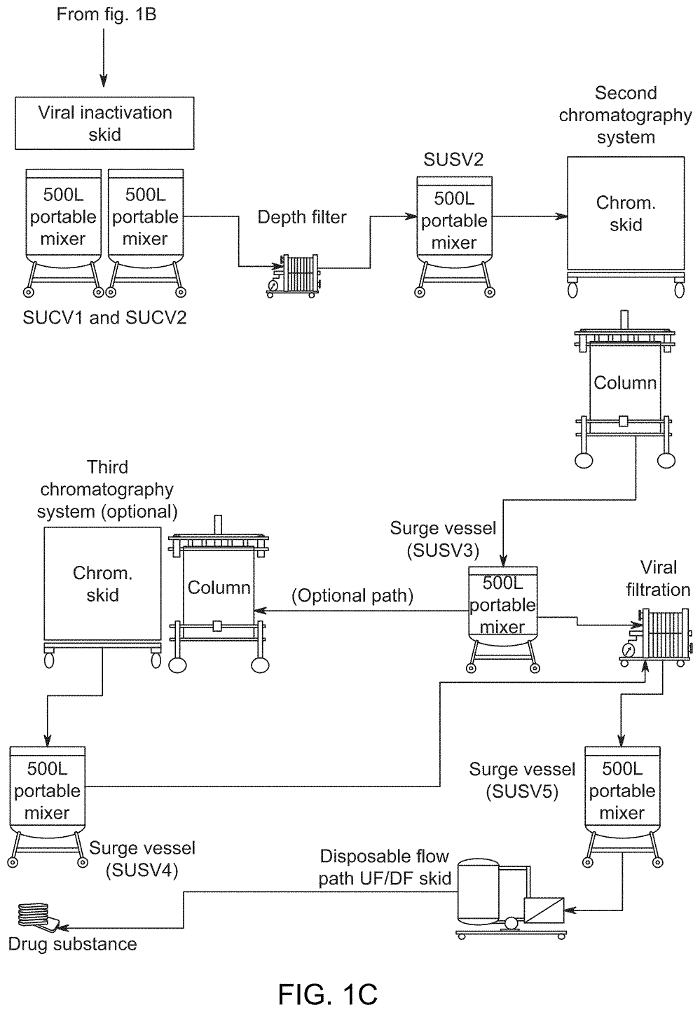

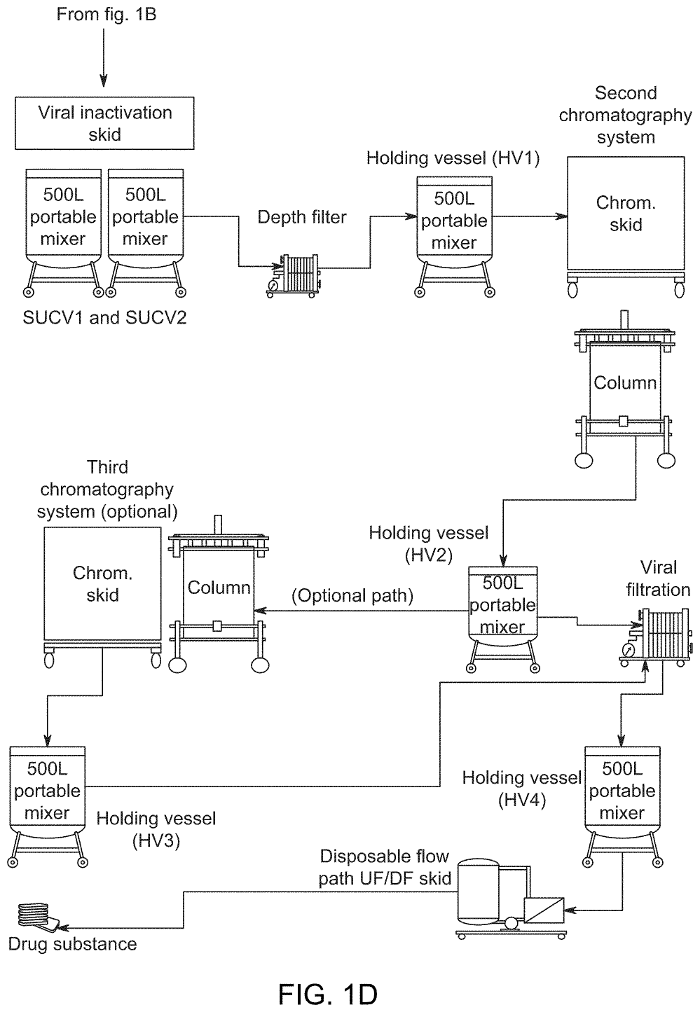

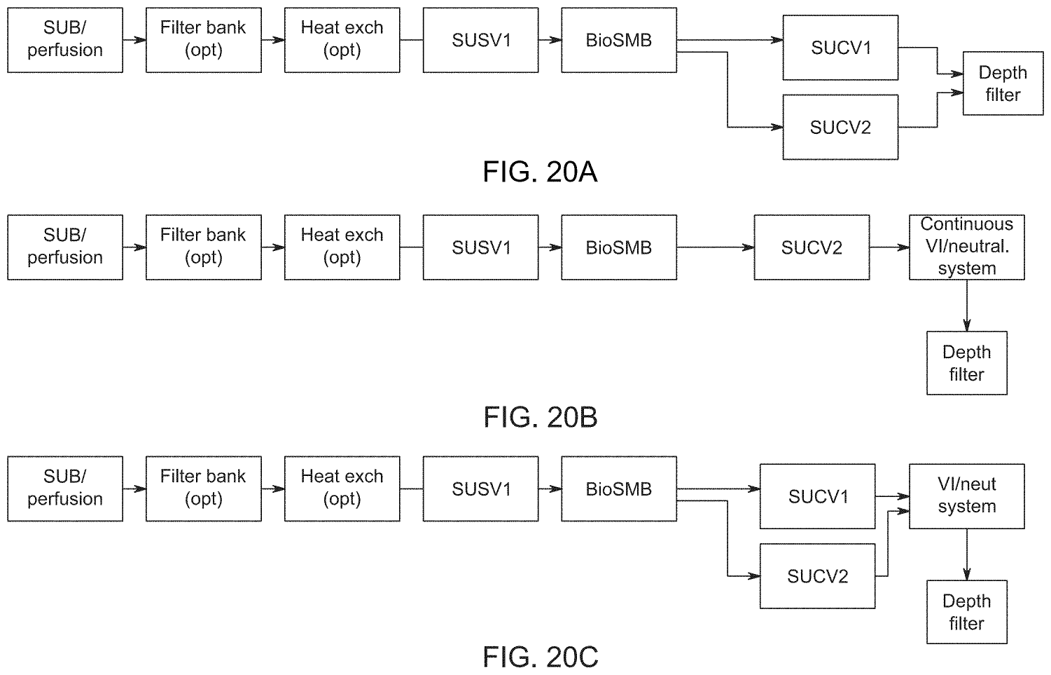

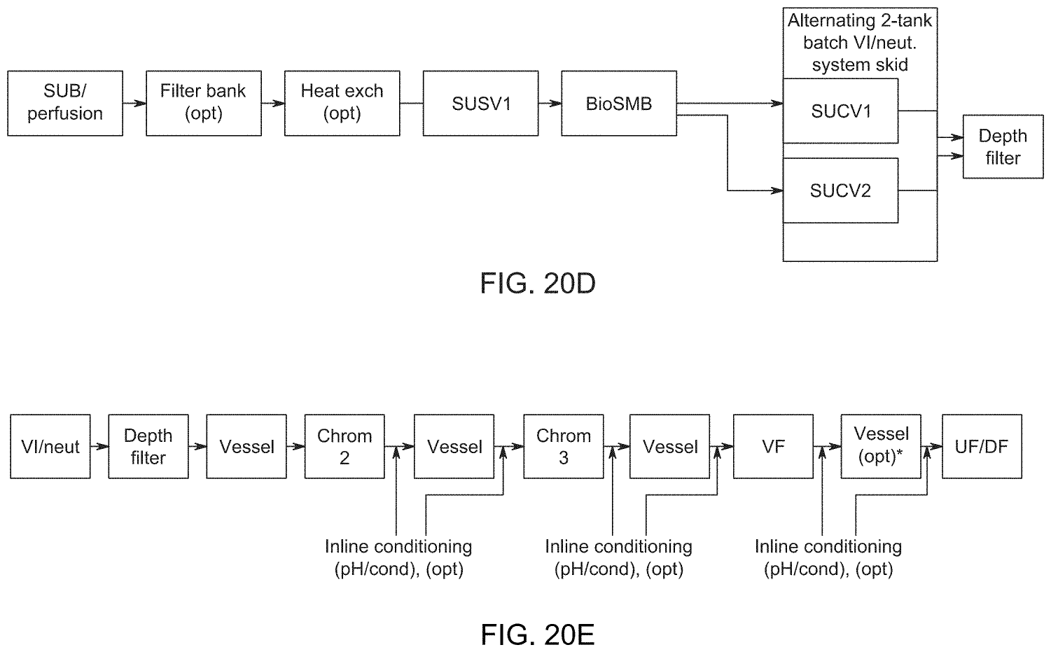

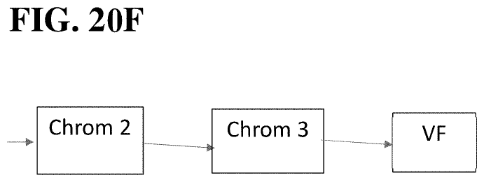

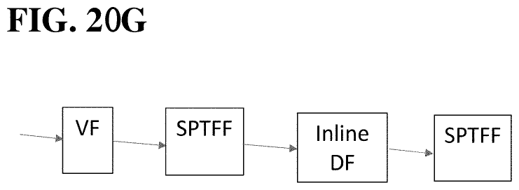

A step of a manufacturing process or a system within an automated manufacturing facility is performed “fluidly,” or is “fluidly connected” to, or “fluidly receives” material from, another step of the manufacturing process or from another system, when material containing the protein of interest flows by pipe, tubing, or other closed conduit between steps or systems without manual loading or unloading. A step of a manufacturing process or a system within an automated manufacturing facility is commonly called a “unit operation.” A unit operation configured to communicate (e.g., by hard-wiring or wireless connection) with an OPC server is called a “batch unit” or “skid.” Typically, but not necessarily, the single-use bioreactor(s), perfusion system, first chromatography system, second chromatography system, optional third chromatography system, and ultrafiltration/diafiltration system are configured as “skids.” (See, e.g., FIGS. 1B-D). The viral inactivation system, and if needed the neutralization system, can also be a skid in some embodiments. (See, e.g., FIG. 20D). A unit operation that is controlled not via hard-wiring, but rather via a dongle and/or a Profibus device, or similar digital information storage device and electronic hardware connector(s), is called a “non-batch unit.” For convenience and flexibility, filter banks, heat exchangers, surge vessels, feed tanks, reservoirs, holding vessels, collection vessels or collection tanks (e.g., an elution collection vessel), and portables mixers and other mixing vessels, when optionally present, are typically configured as non-batch units, although in some embodiments unit operations such as these may also be included in a “skid,” involving control via hard-wiring or wireless connection. (See, e.g., FIG. 5 and FIG. 20D).

The terms “automated,” “automation-controlled,” or “automatically,” are used interchangeably, in connection with a manufacturing process or facility, and refer to computer-control of the implementation or performance of one or more process steps or the operation of a component or system of a manufacturing facility, optionally, with attendant feed-back regulation of the process step or operation. Typically, a computerized controller receives digital signals from detectors of the physical or chemical parameter to be controlled and issues responsive digital instructions to a system or subsystem.

The term “therapeutic protein” means a pharmacologically active protein applicable to the prevention, treatment, or cure of a disease or condition of human beings. Examples of therapeutic proteins include, but are not limited to, monoclonal antibodies, recombinant forms of a native protein (e.g., a receptor, ligand, hormone, enzyme or cytokine), fusion proteins, peptibodies, and/or a monomer domain binding proteins, e.g., based on a domain selected from LDL receptor A-domain, thrombospondin domain, thyroglobulin domain, trefoil/PD domain, VEGF binding domain, EGF domain, Anato domain, Notch/LNR domain, DSL domain, integrin beta domain, and Ca-EGF domain. The preceding are merely exemplary, and a therapeutic protein can comprise any clinically relevant polypeptide target moiety or polypeptide ligand. The term “derivative,” when used in connection with therapeutic proteins of interest, refers to proteins that are covalently modified by conjugation to therapeutic or diagnostic agents, labeling (e.g., with radionuclides or various enzymes), covalent polymer attachment such as PEGylation (derivatization with polyethylene glycol) and insertion or substitution of natural or non-natural amino acids.

A “drug substance” is an active pharmaceutical ingredient (API) intended to furnish pharmacologic activity or other direct effect in the diagnosis, cure, mitigation, treatment, or prevention of disease or to affect the structure or any function of the body. A drug substance can be further formulated, or re-formulated, with buffers, carriers, and/or excipients, and the drug substance can further dosed in a drug product configuration suitable and/or approved for clinical use.

The term “purify” or “purifying” a desired protein means increasing the degree of purity of the desired protein from a composition or solution comprising the protein of interest (i.e., the “POI,” e.g., a therapeutic or other medically useful protein) and one or more contaminants by removing (completely or partially) at least one contaminant from the composition or solution. An “isolated” protein is one that has been identified and separated from one or more components of its natural environment or of a culture medium in which it has been secreted by a producing cell. In some embodiments, the isolated protein is substantially free from proteins or polypeptides or other contaminants that are found in its natural or culture medium environment that would interfere with its therapeutic, diagnostic, prophylactic, research or other use. “Contaminant” components of its natural environment or medium are materials that would interfere with industrial, research, therapeutic, prophylactic, or diagnostic or uses for the protein of interest, and may include enzymes, hormones, and other proteinaceous or nonproteinaceous (e.g., polynucleotides, lipids, carbohydrates) solutes. Typically, an “isolated protein” or, interchangeably, “protein isolate,” constitutes at least about 5%, at least about 10%, at least about 25%, or at least about 50% of a given sample. In some embodiments, the isolated protein of interest will be “purified”: (1) to greater than 95% by weight of protein, and most preferably, more than 99% by weight, or (2) to homogeneity by SDS-PAGE, or other suitable technique, under reducing or nonreducing conditions, optionally using a stain, e.g., Coomassie blue or silver stain. An isolated naturally occurring antibody includes the antibody in situ within recombinant cells since at least one component of the protein's natural environment will not be present. Typically, however, the isolated or purified protein of interest (e.g., a purified protein drug substance) will be prepared by at least one purification step.

A protein of interest, such as a therapeutic or other medically useful protein, for purposes of the present invention, whether it includes a variant or parental antibody amino acid sequence, is typically produced by recombinant expression technology, although it can also be a naturally occurring protein.

“Polypeptide” and “protein” are used interchangeably herein and include a molecular chain of two or more amino acids linked covalently through peptide bonds. The terms do not refer to a specific length of the product. Thus, “peptides,” and “oligopeptides,” are included within the definition of polypeptide. The terms include post-translational modifications of the polypeptide, for example, glycosylations, acetylations, phosphorylations and the like. In addition, protein fragments, analogs, mutated or variant proteins, fusion proteins and the like are included within the meaning of polypeptide. The terms also include molecules in which one or more amino acid analogs or non-canonical or unnatural amino acids are included as can be expressed recombinantly using known protein engineering techniques. In addition, proteins can be derivatized as described herein and by other well-known organic chemistry techniques.

The term peptide or protein “analog” refers to a polypeptide having a sequence that differs from a peptide sequence existing in nature by at least one amino acid residue substitution, internal addition, or internal deletion of at least one amino acid, and/or amino- or carboxy-terminal end truncations, or additions). An “internal deletion” refers to absence of an amino acid from a sequence existing in nature at a position other than the N- or C-terminus. Likewise, an “internal addition” refers to presence of an amino acid in a sequence existing in nature at a position other than the N- or C-terminus.

A “variant” of a polypeptide (e.g., of an immunoglobulin, or an antibody, or a fusion protein) comprises an amino acid sequence wherein one or more amino acid residues are inserted into, deleted from and/or substituted into the amino acid sequence relative to another polypeptide reference sequence. Variants can include variants of fusion proteins.

The term “fusion protein” indicates that the protein includes polypeptide components derived from more than one parental protein or polypeptide. Typically, a fusion protein is expressed from a “fusion gene” in which a nucleotide sequence encoding a polypeptide sequence from one protein is appended in frame with, and optionally separated by a linker from, a nucleotide sequence encoding a polypeptide sequence from a different protein. The fusion gene can then be expressed by a recombinant host cell as a single protein. Fusion proteins incorporating an antibody or an antigen-binding portion thereof are known.

The inventive process involves culturing mammalian cells, e.g., recombinant host cells, capable of producing a secreted protein of interest. A “secreted” protein refers to those proteins capable of being directed to the endoplasmic reticulum (ER), secretory vesicles, or the extracellular space as a result of a secretory signal peptide sequence, as well as those proteins released into the extracellular space without necessarily containing a signal sequence. If the secreted protein is released into the extracellular space, the secreted protein can undergo extracellular processing to produce a “mature” protein. Release into the extracellular space can occur by many mechanisms, including exocytosis and proteolytic cleavage. In some other embodiments, the antibody protein of interest can be synthesized by the host cell as a secreted protein, which can then be further purified from the extracellular space and/or medium.

As used herein “soluble” when in reference to a protein produced by recombinant DNA technology in a host cell is a protein that exists in aqueous solution; if the protein contains a twin-arginine signal amino acid sequence the soluble protein is exported to the periplasmic space in gram negative bacterial hosts, or is secreted into the culture medium by eukaryotic host cells capable of secretion (i.e., “protein-secreting” cells, e.g., protein-secreting mammalian cells), or by bacterial host possessing the appropriate genes (e.g., the kil gene). Thus, a soluble protein is a protein which is not found in an inclusion body inside the host cell. Alternatively, depending on the context, a soluble protein is a protein which is not found integrated in cellular membranes, or, in vitro, is dissolved, or is capable of being dissolved in an aqueous buffer under physiological conditions without forming significant amounts of insoluble aggregates (i.e., forms aggregates less than 10%, and typically less than about 5%, of total protein) when it is suspended without other proteins in an aqueous buffer of interest under physiological conditions, such buffer not containing an ionic detergent or chaotropic agent, such as sodium dodecyl sulfate (SDS), urea, guanidinium hydrochloride, or lithium perchlorate. In contrast, an insoluble protein is one which exists in denatured form inside cytoplasmic granules (called an inclusion body) in the host cell, or again depending on the context, an insoluble protein is one which is present in cell membranes, including but not limited to, cytoplasmic membranes, mitochondrial membranes, chloroplast membranes, endoplasmic reticulum membranes, etc., or in an in vitro aqueous buffer under physiological conditions forms significant amounts of insoluble aggregates (i.e., forms aggregates equal to or more than about 10% of total protein) when it is suspended without other proteins (at physiologically compatible temperature) in an aqueous buffer of interest under physiological conditions, such buffer not containing an ionic detergent or chaotropic agent, such as sodium dodecyl sulfate (SDS), urea, guanidinium hydrochloride, or lithium perchlorate.

A “stable” formulation of a protein is one in which the protein therein essentially retains its physical stability and/or chemical stability and/or biological activity upon processing (e.g., ultrafiltration, diafiltration, other filtering steps, vial filling), transportation, and/or storage of the antibody drug substance and/or drug product. Together, the physical, chemical and biological stability of the protein in a formulation embody the “stability” of the protein formulation, which is specific to the conditions under which the formulated drug product (DP) is stored. For instance, a drug product stored at subzero temperatures would be expected to have no significant change in either chemical, physical or biological activity while a drug product stored at 40° C. would be expected to have changes in its physical, chemical and biological activity with the degree of change dependent on the time of storage for the drug substance or drug product. The configuration of the protein formulation can also influence the rate of change. For instance, aggregate formation is highly influenced by protein concentration with higher rates of aggregation observed with higher protein concentration. Excipients are also known to affect stability of the drug product with, for example, addition of salt increasing the rate of aggregation for some proteins while other excipients such as sucrose are known to decrease the rate of aggregation during storage. Instability is also greatly influenced by pH giving rise to both higher and lower rates of degradation depending on the type of modification and pH dependence.

Various analytical techniques for measuring protein stability are available in the art and are reviewed, e.g., in Wang, W. (1999), Instability, stabilization and formulation of liquid protein pharmaceuticals, Int J Pharm 185:129-188. Stability can be measured at a selected temperature for a selected time period. For rapid screening, for example, the formulation may be kept at 40° C. for 2 weeks to 1 month, at which time stability is measured. Where the formulation is to be stored at 2-8° C., generally the formulation should be stable at 30° C. for at least 1 month, or 40° C. for at least a week, and/or stable at 2-8° C. for at least two years.

A protein “retains its physical stability” in a formulation if it shows minimal signs of changes to the secondary and/or tertiary structure (i.e., intrinsic structure), or aggregation, and/or precipitation and/or denaturation upon visual examination of color and/or clarity, or as measured by UV light scattering or by size exclusion chromatography, or other suitable methods. Physical instability of a protein, i.e., loss of physical stability, can be caused by oligomerization resulting in dimer and higher order aggregates, subvisible, and visible particle formation, and precipitation. The degree of physical degradation can be ascertained using varying techniques depending on the type of degradant of interest. Dimers and higher order soluble aggregates can be quantified using size exclusion chromatography, while subvisible particles may be quantified using light scattering, light obscuration or other suitable techniques.

A protein “retains its chemical stability” in a formulation, if the chemical stability at a given time is such that covalent bonds are not made or broken, resulting in changes to the primary structure of the protein component. Changes to the primary structure may result in modifications of the secondary and/or tertiary and/or quaternary structure of the protein and may result in formation of aggregates or reversal of aggregates already formed. Typical chemical modifications can include isomerization, deamidation, N-terminal cyclization, backbone hydrolysis, methionine oxidation, tryptophan oxidation, histidine oxidation, beta-elimination, disulfide formation, disulfide scrambling, disulfide cleavage, and other changes resulting in changes to the primary structure including D-amino acid formation. Chemical instability, i.e., loss of chemical stability, may be interrogated by a variety of techniques including ion-exchange chromatography, capillary isoelectric focusing, analysis of peptide digests and multiple types of mass spectrometric techniques. Chemical stability can be assessed by detecting and quantifying chemically altered forms of the protein. Chemical alteration may involve size modification (e.g. clipping) which can be evaluated using size exclusion chromatography, SDS-PAGE and/or matrix-assisted laser desorption ionization/time-of-flight mass spectrometry (MALDI/TOF MS), for example. Other types of chemical alteration include charge alteration (e.g. occurring as a result of deamidation) which can be evaluated by charge-based methods, such as, but not limited to, ion-exchange chromatography, capillary isoelectric focusing, or peptide mapping.

Loss of physical and/or chemical stability may result in changes to biological activity as either an increase or decrease of a biological activity of interest, depending on the modification and the protein being modified. A protein “retains its biological activity” in a formulation, if the biological activity of the protein at a given time is within about 30% of the biological activity exhibited at the time the formulation was prepared. Activity is considered decreased if the activity is less than 70% of its starting value. Biological assays may include both in vivo and in vitro based assays such as ligand binding, potency, cell proliferation or other surrogate measure of its biopharmaceutical activity.

The term “naturally occurring,” where it occurs in the specification in connection with biological materials such as polypeptides, nucleic acids, host cells, and the like, refers to materials which are found in nature.

The term “recombinant” indicates that the material (e.g., a nucleic acid or a polypeptide) has been artificially or synthetically (i.e., non-naturally) altered by human intervention. The alteration can be performed on the material within, or removed from, its natural environment or state. For example, a “recombinant nucleic acid” is one that is made by recombining nucleic acids, e.g., during cloning, DNA shuffling or other well known molecular biological procedures. Examples of such molecular biological procedures are found in Maniatis et al., Molecular Cloning. A Laboratory Manual. Cold Spring Harbor Laboratory, Cold Spring Harbor, N.Y. (1982). A “recombinant DNA molecule,” is comprised of segments of DNA joined together by means of such molecular biological techniques.

The term “recombinant protein” or “recombinant polypeptide” as used herein refers to a protein molecule, e.g., a protein of interest, which is expressed using a recombinant DNA molecule. A “recombinant host cell” is a cell that contains and/or expresses a recombinant nucleic acid.

The term “control sequence” or “control signal” refers to a polynucleotide sequence that can, in a particular host cell, affect the expression and processing of coding sequences to which it is ligated. The nature of such control sequences may depend upon the host organism. In particular embodiments, control sequences for prokaryotes may include a promoter, a ribosomal binding site, and a transcription termination sequence. Control sequences for eukaryotes may include promoters comprising one or a plurality of recognition sites for transcription factors, transcription enhancer sequences or elements, polyadenylation sites, and transcription termination sequences. Control sequences can include leader sequences and/or fusion partner sequences. Promoters and enhancers consist of short arrays of DNA that interact specifically with cellular proteins involved in transcription (Maniatis, et al., Science 236:1237 (1987)). Promoter and enhancer elements have been isolated from a variety of eukaryotic sources including genes in yeast, insect and mammalian cells and viruses (analogous control elements, i.e., promoters, are also found in prokaryotes). The selection of a particular promoter and enhancer depends on what cell type is to be used to express the protein of interest. Some eukaryotic promoters and enhancers have a broad host range while others are functional in a limited subset of cell types (for review see Voss, et al., Trends Biochem. Sci., 11:287 (1986) and Maniatis, et al., Science 236:1237 (1987)).

A “promoter” is a region of DNA including a site at which RNA polymerase binds to initiate transcription of messenger RNA by one or more downstream structural genes. Promoters are located near the transcription start sites of genes, on the same strand and upstream on the DNA (towards the 5′ region of the sense strand). Promoters are typically about 100-1000 bp in length.

An “enhancer” is a short (50-1500 bp) region of DNA that can be bound with one or more activator proteins (transcription factors) to activate transcription of a gene.

The terms “in operable combination”, “in operable order” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced. For example, a control sequence in a vector that is “operably linked” to a protein coding sequence is ligated thereto so that expression of the protein coding sequence is achieved under conditions compatible with the transcriptional activity of the control sequences.

The term “polynucleotide” or “nucleic acid” includes both single-stranded and double-stranded nucleotide polymers containing two or more nucleotide residues. The nucleotide residues comprising the polynucleotide can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phosphoraniladate and phosphoroamidate.

The term “oligonucleotide” means a polynucleotide comprising 200 or fewer nucleotide residues. In some embodiments, oligonucleotides are 10 to 60 bases in length. In other embodiments, oligonucleotides are 12, 13, 14, 15, 16, 17, 18, 19, or 20 to 40 nucleotides in length. Oligonucleotides may be single stranded or double stranded, e.g., for use in the construction of a mutant gene. Oligonucleotides may be sense or antisense oligonucleotides. An oligonucleotide can include a label, including a radiolabel, a fluorescent label, a hapten or an antigenic label, for detection assays. Oligonucleotides may be used, for example, as PCR primers, cloning primers or hybridization probes.

A “polynucleotide sequence” or “nucleotide sequence” or “nucleic acid sequence,” as used interchangeably herein, is the primary sequence of nucleotide residues in a polynucleotide, including of an oligonucleotide, a DNA, and RNA, a nucleic acid, or a character string representing the primary sequence of nucleotide residues, depending on context. From any specified polynucleotide sequence, either the given nucleic acid or the complementary polynucleotide sequence can be determined. Included are DNA or RNA of genomic or synthetic origin which may be single- or double-stranded, and represent the sense or antisense strand. Unless specified otherwise, the left-hand end of any single-stranded polynucleotide sequence discussed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences;” sequence regions on the DNA strand having the same sequence as the RNA transcript that are 3′ to the 3′ end of the RNA transcript are referred to as “downstream sequences.”

As used herein, an “isolated nucleic acid molecule” or “isolated nucleic acid sequence” is a nucleic acid molecule that is either (1) identified and separated from at least one contaminant nucleic acid molecule with which it is ordinarily associated in the natural source of the nucleic acid or (2) cloned, amplified, tagged, or otherwise distinguished from background nucleic acids such that the sequence of the nucleic acid of interest can be determined. An isolated nucleic acid molecule is other than in the form or setting in which it is found in nature. However, an isolated nucleic acid molecule includes a nucleic acid molecule contained in cells that ordinarily express the immunoglobulin (e.g., antibody) where, for example, the nucleic acid molecule is in a chromosomal location different from that of natural cells.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of ribonucleotides along the mRNA chain, and also determines the order of amino acids along the polypeptide (protein) chain. The DNA sequence thus codes for the RNA sequence and for the amino acid sequence.

The term “gene” is used broadly to refer to any nucleic acid associated with a biological function. Genes typically include coding sequences and/or the regulatory sequences required for expression of such coding sequences. The term “gene” applies to a specific genomic or recombinant sequence, as well as to a cDNA or mRNA encoded by that sequence. Genes also include non-expressed nucleic acid segments that, for example, form recognition sequences for other proteins. Non-expressed regulatory sequences including transcriptional control elements to which regulatory proteins, such as transcription factors, bind, resulting in transcription of adjacent or nearby sequences.

“Expression of a gene” or “expression of a nucleic acid” means transcription of DNA into RNA (optionally including modification of the RNA, e.g., splicing), translation of RNA into a polypeptide (possibly including subsequent post-translational modification of the polypeptide), or both transcription and translation, as indicated by the context.

An expression cassette is a typical feature of recombinant expression technology. The expression cassette includes a gene encoding a protein of interest, e.g., a gene encoding an antibody sequence, such as an immunoglobulin light chain and/or heavy chain sequence. A eukaryotic “expression cassette” refers to the part of an expression vector that enables production of protein in a eukaryotic cell, such as a mammalian cell. It includes a promoter, operable in a eukaryotic cell, for mRNA transcription, one or more gene(s) encoding protein(s) of interest and a mRNA termination and processing signal. An expression cassette can usefully include among the coding sequences, a gene useful as a selective marker. In the expression cassette promoter is operably linked 5′ to an open reading frame encoding an exogenous protein of interest; and a polyadenylation site is operably linked 3′ to the open reading frame. Other suitable control sequences can also be included as long as the expression cassette remains operable. The open reading frame can optionally include a coding sequence for more than one protein of interest.

As used herein the term “coding region” or “coding sequence” when used in reference to a structural gene refers to the nucleotide sequences which encode the amino acids found in the nascent polypeptide as a result of translation of an mRNA molecule. The coding region is bounded, in eukaryotes, on the 5′ side by the nucleotide triplet “ATG” which encodes the initiator methionine and on the 3′ side by one of the three triplets which specify stop codons (i.e., TAA, TAG, TGA).

Recombinant expression technology typically involves the use of a recombinant expression vector comprising an expression cassette and a mammalian host cell comprising the recombinant expression vector with the expression cassette or at least the expression cassette, which may for example, be integrated into the host cell genome.

The term “vector” means any molecule or entity (e.g., nucleic acid, plasmid, bacteriophage or virus) used to transfer protein coding information into a host cell.

The term “expression vector” or “expression construct” as used herein refers to a recombinant DNA molecule containing a desired coding sequence and appropriate nucleic acid control sequences necessary for the expression of the operably linked coding sequence in a particular host cell. An expression vector can include, but is not limited to, sequences that affect or control transcription, translation, and, if introns are present, affect RNA splicing of a coding region operably linked thereto. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired. Such techniques are well known in the art. (See, e.g., Goodey, Andrew R.; et al., Peptide and DNA sequences, U.S. Pat. No. 5,302,697; Weiner et al., Compositions and methods for protein secretion, U.S. Pat. Nos. 6,022,952 and 6,335,178; Uemura et al., Protein expression vector and utilization thereof, U.S. Pat. No. 7,029,909; Ruben et al., 27 human secreted proteins, US 2003/0104400 A1). For expression of multi-subunit proteins of interest, separate expression vectors in suitable numbers and proportions, each containing a coding sequence for each of the different subunit monomers, can be used to transform a host cell. In other embodiments, a single expression vector can be used to express the different subunits of the protein of interest.

The term “host cell” means a cell that has been transformed, or is capable of being transformed, with a nucleic acid and thereby expresses a gene or coding sequence of interest. The term includes the progeny of the parent cell, whether or not the progeny is identical in morphology or in genetic make-up to the original parent cell, so long as the gene of interest is present. Any of a large number of available and well-known host cells may be used in the practice of this invention to obtain antibody variants, although mammalian host cells capable of post-translationally glycosylating antibodies are preferred. The selection of a particular host is dependent upon a number of factors recognized by the art. These include, for example, compatibility with the chosen expression vector, toxicity of the peptides encoded by the DNA molecule, rate of transformation, ease of recovery of the peptides, expression characteristics, bio-safety and costs. A balance of these factors must be struck with the understanding that not all hosts may be equally effective for the expression of a particular DNA sequence. Modifications can be made at the DNA level, as well. The peptide-encoding DNA sequence may be changed to codons more compatible with the chosen host cell. Codons can be substituted to eliminate restriction sites or to include silent restriction sites, which may aid in processing of the DNA in the selected host cell. Next, the transformed host is cultured and purified. Host cells may be cultured under conventional fermentation conditions so that the desired compounds are expressed. Such fermentation conditions are well known in the art.

Within these general guidelines, microbial host cells in culture, such as bacteria (such as Escherichia coli sp.), and yeast cell lines (e.g., Saccharomyces, Pichia, Schizosaccharomyces, Kluyveromyces) and other fungal cells, algal or algal-like cells, insect cells, plant cells, that have been modified to incorporate humanized glycosylation pathways, can also be used to produce fully functional glycosylated antibody. However, mammalian (including human) host cells, e.g., CHO cells and HEK-293 cells, are particularly useful in the inventive process.

Examples of useful mammalian host cell lines are Chinese hamster ovary cells, including CHO-K1 cells (e.g., ATCC CCL61), CHO-S, DXB-11, DG-44, and Chinese hamster ovary cells/-DHFR (CHO, Urlaub et al, Proc. Natl. Acad. Sci. USA 77: 4216 (1980)); monkey kidney CV1 line transformed by SV40 (COS-7, ATCC CRL 1651); human embryonic kidney line (293 or 293 cells subcloned for growth in suspension culture (Graham et al, J. Gen Virol. 36: 59 (1977)); baby hamster kidney cells (BHK, ATCC CCL 10); mouse Sertoli cells (TM4, Mather, Biol. Reprod. 23: 243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TRI cells (Mather et al., Annals N.Y Acad. Sci. 383: 44-68 (1982)); MRC 5 cells or FS4 cells; or mammalian myeloma cells, e.g., NSO or sp2/0 mouse myeloma cells.

“Cell,” “cell line,” and “cell culture” are often used interchangeably and all such designations herein include cellular progeny. For example, a cell “derived” from a CHO cell is a cellular progeny of a Chinese Hamster Ovary cell, which may be removed from the original primary cell parent by any number of generations, and which can also include a transformant progeny cell. Transformants and transformed cells include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included.

Host cells are transformed or transfected with the above-described nucleic acids or vectors for production of polypeptides (including antigen binding proteins, such as antibodies) and are cultured in conventional nutrient media modified as appropriate for inducing promoters, selecting transformants, or amplifying the genes encoding the desired sequences. In addition, novel vectors and transfected cell lines with multiple copies of transcription units separated by a selective marker are particularly useful for the expression of polypeptides, such as antibodies.

The term “transfection” means the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al., 1981, Gene 13:197. Such techniques can be used to introduce one or more exogenous DNA moieties into suitable host cells.

The term “transformation” refers to a change in a cell's genetic characteristics, and a cell has been transformed when it has been modified to contain new DNA or RNA. For example, a cell is transformed where it is genetically modified from its native state by introducing new genetic material via transfection, transduction, or other techniques. Following transfection or transduction, the transforming DNA may recombine with that of the cell by physically integrating into a chromosome of the cell, or may be maintained transiently as an episomal element without being replicated, or may replicate independently as a plasmid. A cell is considered to have been “stably transformed” when the transforming DNA is replicated with the division of the cell.

The inventive process involves culturing mammalian cells in one or more single-use perfusion bioreactors comprising a liquid culture medium under conditions that allow the cells to secrete the protein of interest into the medium for a production cultivation period of at least 10 days.

Mammalian cells, such as CHO and BHK cells, are generally cultured as suspension cultures. That is to say, the cells are suspended in a liquid cell culture medium, rather than adhering to a solid support. Another useful mode of production is a hollow fiber bioreactor with an adherent cell line. Porous microcarriers can be suitable and are available commercially, sold under brands, such as Cytoline®, Cytopore® or Cytodex® (GE Healthcare Biosciences).

A “cell culture” means the extracellular culture medium (fresh or conditioned) and the mammalian cells cultured therein.

“Cell culture medium” or “culture medium,” used interchangeably herein, is a sterile aqueous medium suitable for growth of cells, and preferably animal cells, more preferably mammalian cells (e.g., CHO cells), in in vitro cell culture. “Feed medium” is fresh cell culture medium added to a cell culture after inoculation of the cells into the cell culture medium and cell growth has been commenced.

The term “production cultivation period” means the period during which protein-secreting mammalian cells are kept under incubation conditions in the bioreactor(s) which physiologically permit the continued production of the protein of interest. In some embodiments, expression of the protein can be constitutive; in other embodiments, expression of the protein can be engineered to be inducible (e.g., TetO-regulated expression). With such inducible expression, the production cultivation period includes only the period of cultivation in the bioreactor(s) when the inducer molecule (e.g., tetracycline, doxycycline, or other tetracycline analog) is present in the culture medium in sufficient quantities to induce expression of the protein of interest. For purposes of the claimed method, the production cultivation period is at least 10 days, or more, or at least 20 days, or more, e.g., 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more; or 10-20 days, or more, or 20-30 days, or more, or 30-45 days, or more, or 45-60 days, or more, or 60-75 days, or more.

During the production cultivation period, fresh sterile liquid culture medium is automatically added into the one or more perfusion bioreactors, mixed contemporaneously from a plurality of different concentrated medium component solutions and an aqueous diluent. The phrase “mixed contemporaneously” means that the concentrated medium components and diluent are mixed together to make fresh culture medium, only within a few seconds or minutes (≤2 minutes) of when needed to replace volumes of medium that are removed from each of the perfusion bioreactor(s), either as volumes of permeate or cell bleed. A bioreactor has a characteristic mixing time, based on bioreactor and impeller design, and the agitation rate. For example, The Xcellerex® XDR 500-L SUB has blend time(s) from 30-55 seconds at agitation rates of 95-150 rpm. Shorter blend times are also possible by increasing agitation. A “permeate” is a volume of conditioned cell culture medium which has been filtered by microfiltration to remove all cells and contains the protein of interest. The conditioned medium upstream of the cell-removing microfilter(s), is called the “retentate,” and the conditioned medium downstream of the microfilter(s) is the “permeate,” which emerges from the perfusion system of the perfusion bioreactor and is ready for further processing, e.g., by the first chromatography system. A “cell bleed” is a volume of cell culture, including some cells and culture medium, which is voided from the bioreactor(s) to waste and/or for analysis. The fresh culture medium is added to the bioreactor(s) periodically or continuously, depending on whether the removal of volumes of cell culture from the bioreactor(s) occurs intermittently (i.e., “periodically”) or continuously.

In some embodiments of the inventive process (and facility), the fresh sterile liquid culture medium is added to the one or more perfusion bioreactors, by injecting the plurality of different concentrated component solutions at fixed ratios to one another, directly into the perfusion bioreactor(s), while an aqueous diluent (a suitable buffer or water) is also added at varied ratio(s) relative to the plurality of different concentrated medium component solutions, to maintain a constant culture volume in each perfusion bioreactor(s) (i.e., to account for the volume of permeate or cell bleed that is being removed from each perfusion bioreactor). In other embodiments, the fresh sterile liquid culture medium is added to the one or more perfusion bioreactors, by injecting the plurality of different concentrated component solutions and the aqueous diluent (a suitable buffer or water) at fixed ratios relative to one another, directly into the perfusion bioreactor(s), to maintain a constant culture volume in each perfusion bioreactor(s). In still other embodiments, the fresh sterile liquid culture medium is added to the one or more perfusion bioreactors, by injecting the plurality of different concentrated component solutions and the aqueous diluent (a suitable buffer or water), at fixed ratios relative to one another, into a mixing chamber wherein fresh sterile liquid culture medium is mixed contemporaneously (in a sterile mixing vessel fluidly connected to the bioreactor(s)) before being added to each perfusion bioreactor(s) to maintain a constant culture volume.

The particular ratios at which the medium components and the diluent are suitably mixed will vary depending on the culture medium recipe used and the concentrations of the concentrated medium components stocks used, and the appropriate ratios can be conveniently calculated by the skilled practitioner.

In accordance with the invention, sub-surface addition of the different concentrated medium component solutions and aqueous diluent is preferably avoided. Delivery of all medium component solutions and aqueous diluent on demand, through separate ports, can be accomplished manually (e.g., by pre-set pumping flow rates for with periodic adjustments, as needed), or automatically (e.g., by using a ratio-controlled pumping skid and automation to maintain the culture volume in the perfusion bioreactor).

The term “buffer” or “buffered solution” refers to solutions which resist changes in pH by the action of its conjugate acid-base range. Examples of useful buffers include acetate, MES, citrate, Tris, bis-tris, histidine, arginine, succinate, citrate, glutamate, and lactate, or a combination of two or more of these, or other mineral acid or organic acid buffers; phosphate is another example of a useful buffer. Salts containing sodium, ammonium, and potassium cations are often used in making a buffered solution.

A “domain” or “region” (used interchangeably herein) of a polynucleotide is any portion of the entire polynucleotide, up to and including the complete polynucleotide, but typically comprising less than the complete polynucleotide. A domain can, but need not, fold independently (e.g., DNA hairpin folding) of the rest of the polynucleotide chain and/or be correlated with a particular biological, biochemical, or structural function or location, such as a coding region or a regulatory region.

A “domain” or “region” (used interchangeably herein) of a protein is any portion of the entire protein, up to and including the complete protein, but typically comprising less than the complete protein. A domain can, but need not, fold independently of the rest of the protein chain and/or be correlated with a particular biological, biochemical, or structural function or location (e.g., a ligand binding domain, or a cytosolic, transmembrane or extracellular domain).

Quantification of the protein of interest, is often useful or necessary to track production and yield, or appropriately formulate the protein or drug substance for further processing or storage. An antibody that specifically binds a domain of the protein of interest, particularly a specific monoclonal antibody, can therefore be useful for these purposes.

The term “antibody”, or interchangeably “Ab”, is used in the broadest sense and includes fully assembled antibodies, monoclonal antibodies (including human, humanized or chimeric antibodies), polyclonal antibodies, multispecific antibodies (e.g., bispecific antibodies), and antibody fragments that can bind antigen (e.g., Fab, Fab′, F(ab′)2, Fv, single chain antibodies, diabodies), comprising complementarity determining regions (CDRs) of the foregoing as long as they exhibit the desired biological activity. Multimers or aggregates of intact molecules and/or fragments, including chemically derivatized antibodies, are contemplated. Antibodies of any isotype class or subclass, including IgG, IgM, IgD, IgA, and IgE, IgG1, IgG2, IgG3, IgG4, IgA1 and IgA2, or any allotype, are contemplated. Different isotypes have different effector functions; for example, IgG1 and IgG3 isotypes have antibody-dependent cellular cytotoxicity (ADCC) activity.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies that are antigen binding proteins are highly specific binders, being directed against an individual antigenic site or epitope, in contrast to polyclonal antibody preparations that typically include different antibodies directed against different epitopes. Nonlimiting examples of monoclonal antibodies include murine, rabbit, rat, chicken, chimeric, humanized, or human antibodies, fully assembled antibodies, multispecific antibodies (including bispecific antibodies), antibody fragments that can bind an antigen (including, Fab, Fab′, F(ab)₂, Fv, single chain antibodies, diabodies), maxibodies, nanobodies, and recombinant peptides comprising CDRs of the foregoing as long as they exhibit the desired biological activity, or variants or derivatives thereof.

The modifier “monoclonal” indicates the character of the antibody as being obtained from a substantially homogeneous population of antibodies, and is not to be construed as requiring production of the antibody by any particular method. For example, monoclonal antibodies may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The term “immunoglobulin” encompasses full or partial antibodies comprising two dimerized heavy chains (HC), each covalently linked to a light chain (LC); a single undimerized immunoglobulin heavy chain and covalently linked light chain (HC+LC), or a chimeric immunoglobulin (light chain+heavy chain)-Fc heterotrimer (a so-called “hemibody”), or a fusion protein comprising a dimerized or undimerized Fc domain, e.g. a peptibody. An “immunoglobulin” is a protein, but is not necessarily an antigen binding protein, e.g., a carrier antibody which is covalently linked to a clinically relevant target-binding moiety. On the other hand, an immunoglobulin can be designed to be bispecific or polyspecific binders of multiple clinically relevant targets. The term “peptibody” refers to a fusion protein molecule comprising an antibody Fc domain (i.e., at least the C_{H}2 and C_{H}3 antibody domains) that excludes antibody C_{H}1, CL, VH, and VL domains as well as Fab and F(ab)₂, wherein the Fc domain is attached to one or more peptides, preferably a pharmacologically active peptide. The production of peptibodies is generally described in PCT publication WO00/24782.

In an “antibody”, each tetramer is composed of two identical pairs of polypeptide chains, each pair having one “light” chain of about 220 amino acids (about 25 kDa) and one “heavy” chain of about 440 amino acids (about 50-70 kDa). The amino-terminal portion of each chain includes a “variable” (“V”) region of about 100 to 110 or more amino acids primarily responsible for antigen recognition. The carboxy-terminal portion of each chain defines a constant region primarily responsible for effector function. The variable region differs among different antibodies. The constant region is the same among different antibodies. Within the variable region of each heavy or light chain, there are three hypervariable subregions that help determine the antibody's specificity for antigen in the case of an antibody that is an antigen binding protein. The variable domain residues between the hypervariable regions are called the framework residues and generally are somewhat homologous among different antibodies. Immunoglobulins can be assigned to different classes depending on the amino acid sequence of the constant domain of their heavy chains. Human light chains are classified as kappa (.kappa.) and lambda (.lamda.) light chains. Within light and heavy chains, the variable and constant regions are joined by a “J” region of about 12 or more amino acids, with the heavy chain also including a “D” region of about 10 more amino acids. See generally, Fundamental Immunology, Ch. 7 (Paul, W., ed., 2nd ed. Raven Press, N.Y. (1989)). An “antibody” also encompasses a recombinantly made antibody, and antibodies that are glycosylated or lacking glycosylation.

The term “light chain” or “immunoglobulin light chain” includes a full-length light chain and fragments thereof having sufficient variable region sequence to confer binding specificity. A full-length light chain includes a variable region domain, V_{L}, and a constant region domain, C_{L}. The variable region domain of the light chain is at the amino-terminus of the polypeptide. Light chains include kappa chains and lambda chains.

The term “heavy chain” or “immunoglobulin heavy chain” includes a full-length heavy chain and fragments thereof having sufficient variable region sequence to confer binding specificity. A full-length heavy chain includes a variable region domain, V_{H}, and three constant region domains, C_{H1}, C_{H2}, and C_{H3}. The V_{H} domain is at the amino-terminus of the polypeptide, and the C_{H} domains are at the carboxyl-terminus, with the C_{H3} being closest to the carboxy-terminus of the polypeptide. Heavy chains are classified as mu (μ), delta (δ), gamma (γ), alpha (α), and epsilon (ε), and define the antibody's isotype as IgM, IgD, IgG, IgA, and IgE, respectively. Heavy chains may be of any isotype, including IgG (including IgG1, IgG2, IgG3 and IgG4 subtypes), IgA (including IgA1 and IgA2 subtypes), IgM and IgE. Several of these may be further divided into subclasses or isotypes, e.g. IgG1, IgG2, IgG3, IgG4, IgA1 and IgA2. Different IgG isotypes may have different effector functions (mediated by the Fc region), such as antibody-dependent cellular cytotoxicity (ADCC) and complement-dependent cytotoxicity (CDC). In ADCC, the Fc region of an antibody binds to Fc receptors (Fc.gamma.Rs) on the surface of immune effector cells such as natural killers and macrophages, leading to the phagocytosis or lysis of the targeted cells. In CDC, the antibodies kill the targeted cells by triggering the complement cascade at the cell surface.

An “Fc region”, or used interchangeably herein, “Fc domain” or “immunoglobulin Fc domain”, contains two heavy chain fragments, which in a full antibody comprise the C_{H1} and C_{H2} domains of the antibody. The two heavy chain fragments are held together by two or more disulfide bonds and by hydrophobic interactions of the C_{H3} domains.

The term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

For a detailed description of the structure and generation of antibodies, see Roth, D. B., and Craig, N. L., Cell, 94:411-414 (1998), herein incorporated by reference in its entirety. Briefly, the process for generating DNA encoding the heavy and light chain immunoglobulin sequences occurs primarily in developing B-cells. Prior to the rearranging and joining of various immunoglobulin gene segments, the V, D, J and constant (C) gene segments are found generally in relatively close proximity on a single chromosome. During B-cell-differentiation, one of each of the appropriate family members of the V, D, J (or only V and J in the case of light chain genes) gene segments are recombined to form functionally rearranged variable regions of the heavy and light immunoglobulin genes. This gene segment rearrangement process appears to be sequential. First, heavy chain D-to-J joints are made, followed by heavy chain V-to-DJ joints and light chain V-to-J joints. In addition to the rearrangement of V, D and J segments, further diversity is generated in the primary repertoire of immunoglobulin heavy and light chains by way of variable recombination at the locations where the V and J segments in the light chain are joined and where the D and J segments of the heavy chain are joined. Such variation in the light chain typically occurs within the last codon of the V gene segment and the first codon of the J segment. Similar imprecision in joining occurs on the heavy chain chromosome between the D and J_{H} segments and may extend over as many as 10 nucleotides. Furthermore, several nucleotides may be inserted between the D and J_{H} and between the V_{H} and D gene segments which are not encoded by genomic DNA. The addition of these nucleotides is known as N-region diversity. The net effect of such rearrangements in the variable region gene segments and the variable recombination which may occur during such joining is the production of a primary antibody repertoire.

The term “hypervariable” region refers to the amino acid residues of an antibody which are responsible for antigen-binding. The hypervariable region comprises amino acid residues from a complementarity determining region or CDR (i.e., residues 24-34 (L1), 50-56 (L2) and 89-97 (L3) in the light chain variable domain and 31-35 (H1), 50-65 (H2) and 95-102 (H3) in the heavy chain variable domain as described by Kabat et al., Sequences of Proteins of Immunological Interest, th Ed. Public Health Service, National Institutes of Health, Bethesda, Md. (1991)). Even a single CDR may recognize and bind antigen, although with a lower affinity than the entire antigen binding site containing all of the CDRs.

An alternative definition of residues from a hypervariable “loop” is described by Chothia et al., J. Mol. Biol. 196: 901-917 (1987) as residues 26-32 (L1), 50-52 (L2) and 91-96 (L3) in the light chain variable domain and 26-32 (H1), 53-55 (H2) and 96-101 (H3) in the heavy chain variable domain.

“Framework” or “FR” residues are those variable region residues other than the hypervariable region residues.

The protein of interest can also be or include one or more antibody fragments. “Antibody fragments” comprise a portion of an intact full length antibody, preferably the antigen binding or variable region of the intact antibody. Examples of antibody fragments include Fab, Fab′, F(ab′)2, and Fv fragments; diabodies; linear antibodies (Zapata et al., Protein Eng., 8(10):1057-1062 (1995)); single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment which contains the constant region. The Fab fragment contains all of the variable domain, as well as the constant domain of the light chain and the first constant domain (CH1) of the heavy chain. The Fc fragment displays carbohydrates and is responsible for many antibody effector functions (such as binding complement and cell receptors), that distinguish one class of antibody from another.

Pepsin treatment yields an F(ab′)2 fragment that has two “Single-chain Fv” or “scFv” antibody fragments comprising the V_{H} and V_{L} domains of antibody, wherein these domains are present in a single polypeptide chain. Fab fragments differ from Fab′ fragments by the inclusion of a few additional residues at the carboxy terminus of the heavy chain CH1 domain including one or more cysteines from the antibody hinge region. Preferably, the Fv polypeptide further comprises a polypeptide linker between the VH and VL domains that enables the Fv to form the desired structure for antigen binding. For a review of scFv see Pluckthun in The Pharmacology of Monoclonal Antibodies, vol. 113, Rosenburg and Moore eds., Springer-Verlag, New York, pp. 269-315 (1994).

A “Fab fragment” is comprised of one light chain and the C_{H1} and variable regions of one heavy chain. The heavy chain of a Fab molecule cannot form a disulfide bond with another heavy chain molecule.

A “Fab′ fragment” contains one light chain and a portion of one heavy chain that contains the V_{H} domain and the C_{H1} domain and also the region between the C_{H1} and C_{H2} domains, such that an interchain disulfide bond can be formed between the two heavy chains of two Fab′ fragments to form an F(ab′)₂ molecule.

A “F(ab′)₂ fragment” contains two light chains and two heavy chains containing a portion of the constant region between the C_{H1} and C_{H2} domains, such that an interchain disulfide bond is formed between the two heavy chains. A F(ab′)₂ fragment thus is composed of two Fab′ fragments that are held together by a disulfide bond between the two heavy chains.

“Fv” is the minimum antibody fragment that contains a complete antigen recognition and binding site. This region consists of a dimer of one heavy- and one light-chain variable domain in tight, non-covalent association. It is in this configuration that the three CDRs of each variable domain interact to define an antigen binding site on the surface of the VH VL dimer. A single variable domain (or half of an Fv comprising only three CDRs specific for an antigen) has the ability to recognize and bind antigen, although at a lower affinity than the entire binding site.

“Single-chain antibodies” are Fv molecules in which the heavy and light chain variable regions have been connected by a flexible linker to form a single polypeptide chain, which forms an antigen-binding region. Single chain antibodies are discussed in detail in International Patent Application Publication No. WO 88/01649 and U.S. Pat. Nos. 4,946,778 and 5,260,203, the disclosures of which are incorporated by reference in their entireties.

“Single-chain Fv” or “scFv” antibody fragments comprise the V_{H} and V_{L} domains of antibody, wherein these domains are present in a single polypeptide chain, and optionally comprising a polypeptide linker between the V_{H} and V_{L} domains that enables the Fv to form the desired structure for antigen binding (Bird et al., Science 242:423-426, 1988, and Huston et al., Proc. Nati. Acad. Sci. USA 85:5879-5883, 1988). An “Fd” fragment consists of the V_{H} and C_{H1} domains.

The term “diabodies” refers to small antibody fragments with two antigen-binding sites, which fragments comprise a heavy-chain variable domain (V_{H}) connected to a light-chain variable domain (V_{L}) in the same polypeptide chain (V_{H} V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

A “domain antibody” is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

The term “antigen binding protein” (ABP) includes antibodies or antibody fragments, as defined herein, that specifically bind a target ligand or antigen of interest.

In general, an antigen binding protein, e.g., a protein of interest, such as an immunoglobulin protein, or an antibody or antibody fragment, “specifically binds” to a target ligand or antigen of interest when it has a significantly higher binding affinity for, and consequently is capable of distinguishing, that target ligand or antigen, compared to its affinity for other unrelated proteins, under similar binding assay conditions. Typically, an antigen binding protein is said to “specifically bind” its target antigen when the dissociation constant (K_{D}) is 10⁻⁸ M or lower. The antigen binding protein specifically binds antigen with “high affinity” when the K_{D} is 10⁻⁹ M or lower, and with “very high affinity” when the K_{D} is 10⁻¹⁰ M or lower.

“Antigen binding region” or “antigen binding site” means a portion of a protein that specifically binds a specified target ligand or antigen. For example, that portion of an antigen binding protein that contains the amino acid residues that interact with a target ligand or an antigen and confer on the antigen binding protein its specificity and affinity for the antigen is referred to as “antigen binding region.” In an antibody, an antigen binding region typically includes one or more “complementary binding regions” (“CDRs”). Certain antigen binding regions also include one or more “framework” regions (“FRs”). A “CDR” is an amino acid sequence that contributes to antigen binding specificity and affinity. “Framework” regions can aid in maintaining the proper conformation of the CDRs to promote binding between the antigen binding region and an antigen. In a traditional antibody, the CDRs are embedded within a framework in the heavy and light chain variable region where they constitute the regions responsible for antigen binding and recognition. A variable region of an immunoglobulin antigen binding protein comprises at least three heavy or light chain CDRs, see, supra (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991, supra; see also Chothia and Lesk, 1987, supra).

The term “target” or “antigen” refers to a molecule or a portion of a molecule capable of being bound by a selective binding agent, such as an antigen binding protein (including, e.g., an antibody or immunologically functional fragment of an antibody), and additionally capable of being used in an animal to produce antibodies capable of binding to that antigen. An antigen may possess one or more epitopes that are capable of interacting with different antigen binding proteins, e.g., with antibodies.

The term “epitope” is the portion of a target molecule that is bound by an antigen binding protein (for example, an antibody or antibody fragment). The term includes any determinant capable of specifically binding to an antigen binding protein, such as an antibody or to a T-cell receptor. An epitope can be contiguous or non-contiguous (e.g., in a single-chain polypeptide, amino acid residues that are not contiguous to one another in the polypeptide sequence but that within the context of the molecule are bound by the antigen binding protein). In certain embodiments, epitopes may be mimetic in that they comprise a three-dimensional structure that is similar to an epitope used to generate the antigen binding protein, yet comprise none or only some of the amino acid residues found in that epitope used to generate the antigen binding protein. Most often, epitopes reside on proteins, but in some instances may reside on other kinds of molecules, such as nucleic acids. Epitope determinants may include chemically active surface groupings of molecules such as amino acids, sugar side chains, phosphoryl or sulfonyl groups, and may have specific three dimensional structural characteristics, and/or specific charge characteristics. Generally, antigen binding proteins specific for a particular target will preferentially recognize an epitope on the target in a complex mixture of proteins and/or macromolecules.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) must be addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073. For example, sequence identity can be determined by standard methods that are commonly used to compare the similarity in position of the amino acids of two polypeptides. Using a computer program such as BLAST or FASTA, two polypeptide or two polynucleotide sequences are aligned for optimal matching of their respective residues (either along the full length of one or both sequences, or along a pre-determined portion of one or both sequences). The programs provide a default opening penalty and a default gap penalty, and a scoring matrix such as PAM 250 (a standard scoring matrix; see Dayhoff et al., in Atlas of Protein Sequence and Structure, vol. 5, supp. 3 (1978)) can be used in conjunction with the computer program. For example, the percent identity can then be calculated as: the total number of identical matches multiplied by 100 and then divided by the sum of the length of the longer sequence within the matched span and the number of gaps introduced into the longer sequences in order to align the two sequences. In calculating percent identity, the sequences being compared are aligned in a way that gives the largest match between the sequences.

The GCG program package is a computer program that can be used to determine percent identity, which package includes GAP (Devereux et al., 1984, Nucl. Acid Res. 12:387; Genetics Computer Group, University of Wisconsin, Madison, Wis.). The computer algorithm GAP is used to align the two polypeptides or two polynucleotides for which the percent sequence identity is to be determined. The sequences are aligned for optimal matching of their respective amino acid or nucleotide (the “matched span”, as determined by the algorithm). A gap opening penalty (which is calculated as 3.times. the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm.

Recommended parameters for determining percent identity for polypeptides or nucleotide sequences using the GAP program include the following:

Algorithm: Needleman et al., 1970, J. Mol. Biol. 48:443-453;

Comparison matrix: BLOSUM 62 from Henikoff et al., 1992, supra;

Gap Penalty: 12 (but with no penalty for end gaps)

Gap Length Penalty: 4

Threshold of Similarity: 0

Certain alignment schemes for aligning two amino acid sequences may result in matching of only a short region of the two sequences, and this small aligned region may have very high sequence identity even though there is no significant relationship between the two full-length sequences. Accordingly, the selected alignment method (GAP program) can be adjusted if so desired to result in an alignment that spans at least 50 contiguous amino acids of the target polypeptide.

The term “modification” when used in connection with proteins of interest, include, but are not limited to, one or more amino acid changes (including substitutions, insertions or deletions); chemical modifications; covalent modification by conjugation to therapeutic or diagnostic agents; labeling (e.g., with radionuclides or various enzymes); covalent polymer attachment such as PEGylation (derivatization with polyethylene glycol) and insertion or substitution by chemical synthesis of non-natural amino acids. By methods known to the skilled artisan, proteins, can be “engineered” or modified for improved target affinity, selectivity, stability, and/or manufacturability before the coding sequence of the “engineered” protein is included in the expression cassette.

The term “switching,” or “switch,” used herein interchangeably), with respect to a protein isolate fraction, a purified product pool, a virus-free filtrate, or another pool, fraction, eluate, or resultant liquid outflow from a process step or facility component, means to direct, shunt, steer, stream, or convey that outflow fluidly into a subsequent process step or facility component. Such switching can be under the automatic control and regulation of a computer and/or robotic mechanism(s) (e.g., valves or pumps), or can be manually controlled and regulated.

The term “switchable” in connection with collection vessels, surge vessels, holding vessels, mixing vessels, tanks, bags, conduits, pipe, tubing, or other conveyance, into or out of, or by which liquids can flow, means that such flow can be switched, directed, shunted, steered, streamed, or conveyed fluidly to a different vessel, tank, conduit, pipe, tubing, or conveyance. Such switching can be under the automatic control and regulation of a computer and/or robotic mechanism(s) (e.g., valves or pumps), or can be manually controlled and regulated.

The term “surge vessel” means a storage reservoir, mixing vessel, feed tank, or collection vessel (or interchangeably, a “collection tank”), at the downstream end of a conduit, feeder, dam, pipe, or tubing, to absorb discrepant flow rates between two fluidly connected unit operations, e.g., the flow rate of a permeate coming from a bioreactor and the flow rate of a first chromatography system under automated control in continuous or semi-continuous format process embodiments of the invention. The surge vessel absorbs changes or differences in flow rates by allowing the volume to surge within pre-set volume range limits between the fluidly connected unit operations (see, e.g., FIG. 3). For purposes of the invention, surges vessels typically contain up to 50-650 L in volume; in semi-continuous process embodiments, 100-L to 650-L vessels are most useful, while in continuous process embodiments, 50-L to 200-L vessels are usually sufficient. In some embodiments of the invention, operations downstream of the viral inactivation system/neutralization system involve batch-wise processing of the virally inactivated product pool (which can optionally also be filtered by depth filtration to yield a filtered virally inactivated product pool (FVIP)); in such embodiments, the virally inactivated product pool is collected in a collection vessel, and in subsequent batch-wise steps or operations, the purified product pool or virus-free filtrate can optionally be collected in other collection vessels between steps. In such discrete operation, batch-wise, or batch mode, processing, the collection vessel(s) or interchangeably “collection tank(s),” from one step (which in certain embodiments may also be deemed a “feed tank(s)” for the subsequent step) lack the automated controls of a surge vessel, and although the collection vessel (or feed tank) may physically resemble a surge vessel, for purposes of the invention such a collection vessel (or interchangeably, “collection tank”) or feed tank, is called a “holding vessel” or, interchangeably an “HV” (e.g., HV1, HV2, HV3, HV4, or HV5). A “holding vessel” can be a single-use holding vessel (SUHV), distinct from a single-use collection vessel (SUCV, e.g., SUCV1 or SUCV2) in a continuous or semi-continuous format set of manufacturing process steps or operations.

A “chromatography system” is an arrangement of at least one enclosed chromatography matrix, with closed conduit hardware (e.g., pipes or tubing) for fluid ingress and egress from the at least one chromatography matrix. The chromatography system involves one or more pumps and/or valves to automatically or manually control the fluid flow rate and pressure. The first, second and third chromatography systems of the inventive process and facility can incorporate chromatography matrices of various sorts, which the skilled practitioner knows how to select and use in sequence, as appropriate for the protein of interest. Encompassed within the term “matrix” are resins, beads, nanoparticles, nanofibers, hydrogels, membranes (e.g., membrane adsorbers (MAs)), and monoliths, or any other physical matrix, bearing a relevant covalently bound chromatographic ligand (e.g., Protein A, Protein G, or other affinity chromatographic ligand, such as a target ligand, a charged moiety, or a hydrophobic moiety, etc.) for purposes of the inventive method. The matrix to which the affinity target ligand is attached is most often agarose, but other matrices are available. For example, mechanically stable matrices such as controlled pore glass, methacrylate (e.g., in Amsphere™ A3 resin; JRS Life Sciences), or poly(styrenedivinyl)benzene allow for greater stability, faster flow rates and shorter processing times than can be achieved with agarose. Where the protein comprises a CH3 immunoglobulin domain, the Bakerbond ABX™ resin (J. T. Baker, Phillipsburg, N.J.) can be useful for purification. An affinity chromatography matrix may be placed or packed into a column useful for the purification of proteins. Loading of the cell-free cell culture fraction onto the affinity chromatography matrix, e.g., in the first chromatography system, preferably occurs at about neutral pH.

The term “to bind” or “binding” a molecule to Protein A, or a Protein A matrix, or another (different) affinity chromatography matrix, means exposing the molecule to the affinity chromatography ligand covalently bound to a solid substrate (e.g., a resin), under appropriate conditions (e.g., pH and selected salt/buffer composition), such that the molecule of interest is reversibly immobilized in, or on, the affinity chromatography ligand by virtue of its binding affinity under those conditions, regardless of the physical mechanism of affinity that may be involved. (See, e.g., Jendeberg, L. et al., The Mechanism of Binding Staphylococcal Protein A to Immunoglobin G Does Not Involve Helix Unwinding, Biochemistry 35(1): 22-31 (1996); Nelson, J. T. et al., Mechanism of Immobilized Protein A Binding to Immunoglobulin G on Nanosensor Array Surfaces, Anal. Chem., 87(16):8186-8193 (2015)).

The term “to bind” or “binding” a molecule to an ion exchange matrix (e.g., a CEX matrix, such as a CEX resin or membrane adsorber, or an AEX matrix, such as an AEX resin or membrane adsorber), means exposing the molecule to the ion exchange matrix under appropriate conditions (e.g., pH and selected salt/buffer composition) such that the molecule is reversibly immobilized in, or on, the ion exchange matrix by virtue of ionic interactions between the molecule and a charged group or charged groups (i.e., charged ligands) of the ion exchange matrix.

The term “loading buffer” or “equilibrium buffer” refers to the buffer, and salt or salts, which is mixed with a protein preparation (e.g., a batch or perfusion cell culture permeate or filtrate, or an eluant pool containing the protein of interest) for loading the protein preparation onto a Protein A matrix or other affinity chromatography matrix, or onto an ion exchange matrix (e.g., a CEX matrix or AEX matrix), or onto a hydrophobic interaction chromatography (HIC) matrix, as the case may be. This buffer is also used to equilibrate the chromatography matrix before loading, and to wash after loading the protein.

The term “wash buffer” is used herein to refer to the buffer that is passed over a Protein A matrix or another affinity chromatography matrix, or ion exchange matrix (e.g., a CEX matrix or AEX matrix), or a hydrophobic interaction chromatography (HIC) matrix, as the case may be, following loading of a protein preparation and prior to elution or after flow-through of the protein of interest. The wash buffer may serve to remove one or more contaminants without substantial elution of the desired protein or can be used to wash out a non-binding protein.

The term “elution buffer” or “eluant” refers to the buffer used to elute the protein of interest reversibly bound to a matrix. As used herein, the term “solution” refers to either a buffered or a non-buffered solution, including water.

The term “elution pool” or “eluant pool” means the material eluted from a matrix, which material includes the recombinant protein of interest.

The term “loading,” with respect to a Protein A matrix or other affinity chromatography matrix, or an ion exchange matrix (e.g., a CEX matrix), or a hydrophobic interaction chromatography (HIC) matrix, means loading a protein preparation (e.g., a batch or perfusion cell culture permeate or filtrate, or an eluant pool containing the protein of interest) onto the Protein A matrix or another affinity chromatography matrix, or the ion exchange matrix, or the HIC matrix.

The term “washing,” with respect to a Protein A matrix or other affinity chromatography matrix, or an ion exchange matrix (e.g., a CEX matrix or AEX matrix), or a HIC matrix, means passing an appropriate buffer through or over the Protein A matrix or ion exchange matrix or HIC matrix or other chromatographic matrix, as the case may be.

The term “eluting” a molecule (e.g. a desired recombinant protein or contaminant) from a Protein A matrix or another affinity chromatography matrix, or an ion exchange matrix (e.g., a CEX matrix or AEX matrix), or an HIC matrix, means removing the molecule from such material, typically by passing an elution buffer over the chromatography matrix.

The terms “single-use” or “single use” component(s), used interchangeably, means that a particular aseptic production line component, i.e., an aseptic piece of equipment, used in the inventive automated facility or in performing the inventive process is constructed or configured to be employed for a single production run (but may be re-used if quality and aseptic sanitation can be assured for multiple runs). The single-use component can then be disposed of and replaced for subsequent production runs by a another single-use component of the same or modified configuration without the need for cleaning and sanitization of the component between production runs. Examples of single-use components that can be employed in the present invention include, but are not limited to, a perfusion bioreactor, the first chromatography system, the second chromatography system, the third chromatography system, the low pH or detergent viral inactivation system, the neutralization system, the viral filtration system, or the ultrafiltration/diafiltration system. Such single-use components can be constructed or obtained commercially, for example, but not limited to the following:

Single-use bioreactors: XCellerex® XDR single-use bioreactor bags (e.g., 500-L, 1000-L, or 2000-L volumes; GE Healthcare Life Sciences); BIOSTAT STR® stirred tank single-use bioreactor systems (e.g., 500-L to 2000-L volumes; Sartorius Stedim Biotech); HyPerforma Single-Use Bioreactors (e.g., 50-L, 100-L, 200-L, 500-L, 1000-L and 2000-L volumes; Thermo Fisher Scientific); Allegro™ Single-Use Stirred Tank Bioreactors (e.g., 500-L to 2000-L volumes; Pall); Millipore Mobius® Single-use Bioreactors (e.g., 500-L to 2000-L volumes; MilliporeSigma),) 50-L Rocking Bioreactor bags, including, but not limited to, Wave Bioreactor® Bag (GE Healthcare Life Sciences) or RIM Bio Rocker Bags; or mixer bags sold commercially by Pall or Sartorius (e.g., 100-L, 200-L, 650-L, 1000-L or 2000-L volumes);

Single-use perfusion systems: Spectrum Krosflo® Hollow Fiber Systems or Repligen Alternating Tangential Flow (ATF-6 and 10) Systems;

Single-use heat exchangers: Thermo Scientific™ DHX™ Heat Exchanger with a Thermo Scientific™ ThermoFlex™ Recirculating Chiller, and Thermo Scientific™ DHX™ Bag Assembly;

Single-use filter assembly systems containing filters (various membrane and pore sizes from MilliporeSigma or Sartorius Stedim Biotech), silicone and/or c-flex tubing, and aseptic connectors (from Pall, Colder, GE Healthcare Life Sciences, Sartorius Stedim Biotech);

Single-use transfer lines of various dimensions, lengths, and configurations using disposable aseptic connectors, silicone and/or c-flex type tubing are commercially available from Thermo Fisher Scientific (ASI) or Advantapure;

Single-use medium component solution or aqueous diluent (e.g., buffer) solution tote storage bags are sold commercially by Advanced Scientifics, Inc. (ASI; Thermo Fisher Scientific), MilliporeSigma, Sartorius, or RIM Bio;

Single-use viral inactivation systems: Cadence® Virus Inactivation System manifolds (Pall Life Sciences), FlexAct® for low pH Virus Inactivation (“VI”; Sartorius); Single-use chromatography systems: Cadence™ BioSMB® PD (Pall Life Sciences); Allegro™ Single Use Chromatography (Pall Biotech); Mobius® FlexReady Chromatography (MilliporeSigma); ÄKTA™ Ready Single Use Chromatography (GE Healthcare Life Sciences); or Sartobind® IEX membrane adsorbers (Sartorius Stedim Biotech);

Single-use viral filtration systems: Allegro™ MVP Single Use System Manifolds (Pall Biotech); Mobius® FlexReady for Viral Filtration (MilliporeSigma); FlexAct® for Viral Filtration (Sartorius), Planova™ Single-Use Virus Filtration (SU-VFS; Asahi Kasei Bioprocess America, Inc.), or Viresolve® Pro Virus Filtration (MilliporeSigma);

Single-use UF/DF systems: Allegro™ Single Use Tangential Flow Filtration System (Pall Biotech); Mobius® FlexReady TFF System (MilliporeSigma); FlexAct® for UF/DF (Sartorius); ÄKTA™ Readyflux single use filtration (GE Healthcare Life Sciences); and

Single-use aseptic connectors: AseptiQuik® connectors (Colder Products Company), Kleenpak® Presto Sterile Connector (Pall Biotech); Lynx® ST Connector (MilliporeSigma).

The term “filter bank” or “filter assembly system”, used interchangeably refers to an apparatus that includes multiple filter assemblies with each filter assembly including at least one filter. A filter included in a filter assembly can be a single-use filter and replaced after a period of time and/or after an amount of use. A filter bank can be a portable piece of equipment. For example, a filter bank can be disposed on a filtration cart that can be moved to various locations in an automated facility. The filters included in a filter bank can include a filtration system comprising a depth filter, a 0.2 micrometer filter, a membrane filter, a 20 nanometer (nm) filter, a viral filtration device, an ultrafiltration device, a diafiltration device, or combinations thereof. A filter bank can be configured such that while material is flowing through at least one filter of the filter bank, another filter of the filter bank remains unused. In various embodiments, a filter bank can be coupled to a diverter valve or other flow control device to control the flow of material to the filters included in the filter bank. The diverter valve or flow control device can be pneumatically controlled.

The foregoing are merely exemplary, and not an exhaustive list, of single-use systems and connectors that are available to the skilled practitioner of the present invention.

Proteins of Interest

The protein of interest to be manufactured using the present invention can be any industrially or medically useful protein, such as, but not limited to, a pharmacologically active protein or peptide.

For example, the protein of interest can be a mimetic or agonist peptide. The terms “-mimetic peptide,” “peptide mimetic,” and “-agonist peptide” refer to a peptide or protein having biological activity comparable to a naturally occurring protein of interest. These terms further include peptides that indirectly mimic the activity of a naturally occurring peptide molecule, such as by potentiating the effects of the naturally occurring molecule.

The protein of interest can be an antagonist peptide or inhibitor peptide. The term “-antagonist peptide,” “peptide antagonist,” and “inhibitor peptide” refer to a peptide or protein that blocks or in some way interferes with the biological activity of a receptor of interest, or has biological activity comparable to a known antagonist or inhibitor of a receptor of interest (such as, but not limited to, an ion channel or a G-Protein Coupled Receptor (GPCR)).

Examples of pharmacologically active proteins that can be manufactured with the present invention include, but are not limited to, an IL-6 binding peptide, a CD3 binding protein, a CD19 binding protein, a CD20 binding protein, a CD22 binding protein, a HER2 binding protein, a HER3 binding protein, a vascular endothelial growth factor-A (VEGF-A) binding protein, a TNF-α binding protein, an EGFR binding protein, a RANK ligand binding protein, an IL-1α binding protein, an IL-1β binding protein, an IL-17A binding protein, an EPCAM (CD326) binding protein, a CGRP peptide antagonist, a bradykinin B1 receptor peptide antagonist, a toxin peptide, a placental growth factor (PIGF) binding protein, a parathyroid hormone (PTH) agonist peptide, a parathyroid hormone (PTH) antagonist peptide, an ang-1 binding peptide, an ang-2 binding peptide, a myostatin binding peptide, an erythropoietin-mimetic (EPO-mimetic) peptide, a FGF21 peptide, a thrombopoietin-mimetic (TPO-mimetic) peptide (e.g., AMP2 or AMPS), a nerve growth factor (NGF) binding peptide, a B cell activating factor (BAFF) binding peptide, and a glucagon-like peptide (GLP)-1 or a peptide mimetic thereof or GLP-2 or a peptide mimetic thereof.

Protein and coding sequences for such proteins, some of which have already received regulatory approval, are well known in the art. However, the present invention can also be applied to the manufacture of drug substances yet to be innovated by methods of drug discovery, research and development, and clinical trials.

Cloning DNA

Cloning of DNA is carried out using standard techniques (see, e.g., Sambrook et al. (1989) Molecular Cloning: A Laboratory Guide, Vols 1-3, Cold Spring Harbor Press, which is incorporated herein by reference). For example, a cDNA library may be constructed by reverse transcription of polyA+mRNA, preferably membrane-associated mRNA, and the library screened using probes specific for human immunoglobulin polypeptide gene sequences. In one embodiment, however, the polymerase chain reaction (PCR) is used to amplify cDNAs (or portions of full-length cDNAs) encoding an immunoglobulin gene segment of interest (e.g., a light or heavy chain variable segment). The amplified sequences can be readily cloned into any suitable vector, e.g., expression vectors, minigene vectors, or phage display vectors. It will be appreciated that the particular method of cloning used is not critical, so long as it is possible to determine the sequence of some portion of the protein of interest.

One source for antibody nucleic acids is a hybridoma produced by obtaining a B cell from an animal immunized with the antigen of interest and fusing it to an immortal cell. Alternatively, nucleic acid can be isolated from B cells (or whole spleen) of the immunized animal. Yet another source of nucleic acids encoding antibodies is a library of such nucleic acids generated, for example, through phage display technology. Polynucleotides encoding peptides of interest, e.g., variable region peptides with desired binding characteristics, can be identified by standard techniques such as panning.

Sequencing of DNA is carried out using standard techniques (see, e.g., Sambrook et al. (1989) Molecular Cloning: A Laboratory Guide, Vols 1-3, Cold Spring Harbor Press, and Sanger, F. et al. (1977) Proc. Natl. Acad. Sci. USA 74: 5463-5467, which is incorporated herein by reference). By comparing the sequence of the cloned nucleic acid with published sequences of genes and cDNAs, one of skill will readily be able to determine, depending on the region sequenced. One source of gene sequence information is the National Center for Biotechnology Information, National Library of Medicine, National Institutes of Health, Bethesda, Md. Gene sequencing can also be done, for example, by standard methods or by so-called “Next-generation” sequencing of engineered DNA constructs prior to transfection. (See, e.g., Buermans, H. P. J., & den Dunnen, J. T., Next generation sequencing technology: Advances and applications, Biochimica et Biophysica Acta—Molecular Basis of Disease 1842(10): 1932-1941 (2014)).

Chemical synthesis of parts or the whole of a coding region containing codons reflecting desires protein changes can be cloned into an expression vector by either restriction digest and ligation of 5′ and 3′ ends of fragments or the entire open reading frame (ORF), containing nucleotide overhangs that are generated by restriction enzyme digestion and which are compatible to the destination vector. The fragments or inserts are typically ligated into the destination vector using a T4 ligase or other common enzyme. Other useful methods are similar to the above except that the cut site for the restriction enzyme is at location different from the recognition sequence. Alternatively, isothermal assembly (i.e., “Gibson Assembly”) can be employed, in which nucleotide overhangs are generated during synthesis of fragments or ORFs; digestion by exonucleases is employed. Alternatively, nucleotide overhangs can be ligated ex vivo by a ligase or polymerase or in vivo by intracellular processes.

Alternatively, homologous recombination can be employed, similar to isothermal assembly, except exonuclease activity of T4 DNA ligase can used on both insert and vector and ligation can be performed in vivo.

Another useful cloning method is the so-called “TOPO” method, in which a complete insert containing a 3′ adenosine overhang (generated by Taq polymerase) is present, and Topoisomerase I ligates the insert into a TOPO vector.

Another useful cloning method is degenerate or error-prone PCR exploiting degenerate primers and/or a thermally stable low-fidelity polymerase caused by the polymerase within certain reaction conditions. Fragments or inserts are then cloned into an expression vector.

The above are merely examples of known cloning techniques, and the skilled practitioner knows how to employ any other suitable cloning techniques.

Isolated DNA can be operably linked to control sequences or placed into expression vectors, which are then transfected into host cells that do not otherwise produce immunoglobulin protein, to direct the synthesis of monoclonal antibodies in the recombinant host cells. Recombinant production of antibodies is well known in the art.

Nucleic acid is operably linked when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, operably linked means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

Many vectors are known in the art. Vector components may include one or more of the following: a signal sequence (that may, for example, direct secretion of the expressed protein by the recombinant host cells); an origin of replication, one or more selective marker genes (that may, for example, confer antibiotic or other drug resistance, complement auxotrophic deficiencies, or supply critical nutrients not available in the media), an enhancer element, a promoter, and a transcription termination sequence, all of which are well known in the art.

Protein Expression

The inventive method for manufacturing a purified protein of interest (e.g., but not limited to, a protein drug substance) involves culturing protein-secreting mammalian cells. Such cultured mammalian cells are typically made by recombinant DNA technology involving transient or stable transfection, e.g., the pooled plasmid constructs (expression vectors) from the cloning step can be transfected into a plurality of host cells (e.g., mammalian, e.g., HEK 293 or CHO, bacterial, insect, yeast cells) for expression using a cationic lipid, polyethylenimine, Lipofectamine™, or ExpiFectamine™, or electroporation. The skilled practitioner is aware of numerous suitable means for transfecting to achieve expression of recombinant antibodies. Alternatively, methods for stable genomic integration of expressions cassettes encoding the protein of interest can be employed to make a production cell line of protein-secreting mammalian cells. (See, e.g., Zhang, Crispr-Cas Systems and Methods for Altering Expression Of Gene Products, WO2014093661 A2; Frendewey et al., Methods and Compositions for the Targeted Modification of a Genome, U.S. Pat. No. 9,228,208 B2; Church et al., Multiplex Automated Genome Engineering, WO2008052101A2, U.S. Pat. No. 8,153,432 B2; Bradley et al., Methods Cells and Organisms, US2015/0079680 A1; Begemann et al., Compositions and Methods for Modifying Genomes, WO2017141173A2; Gill et al., Nucleic acid-guided nucleases, U.S. Pat. No. 9,982,279 B1; Minshull et al., Enhanced nucleic acid constructs for eukaryotic gene expression, U.S. Pat. No. 9,428,767B2, U.S. Pat. No. 9,580,697B2, U.S. Pat. No. 9,574,209B2; Minshull et al., DNA Vectors, Transposons And Transposases For Eukaryotic Genome Modification, U.S. Ser. No. 10/041,077B2).

Optionally, the transfectant or transformant cells will be provided with a recombinant expression cassette for a selectable marker, for example, but not limited to, one or more of the following: glutamine synthase, dihydrofolate reductase, puromycin-N acetyl transferase, blasticidin-S deaminase, hygromycin phosphotransferase, aminoglycoside phosphotransferase, nourseothircin N-acetyl transferase, or a protein that binds to zeocin.

The protein of interest is typically obtained by culturing the transfected or transformed host cells under physiological conditions allowing the cells to express recombinant proteins. Most conveniently, the expressed recombinant proteins are directly secreted into the extracellular culture medium (by employing appropriate secretory-directing signal peptides) and are harvested therefrom; otherwise additional steps will be needed to isolate the expressed antibodies from a cell extract.

The desired scale of the recombinant expression will be dependent on the type of expression system and the desired quantity of protein production. Some expression systems such as ExpiCHO™ usually produce higher yields as compared to some earlier HEK293 technologies. A smaller scale ExpiCHO™ might then suffice as compared to an HEK293 system. Efficiency of transfection can also be a consideration in choosing an appropriate expression system. Electroporation can be a suitable method given its effectiveness, relative low cost and the fact that high-throughput during this step is not critical. Additionally, the ratio of immunoglobulin light chain to heavy chain can be varied during the co-transfection to improve expression of certain variants. The product yield for a given variant has to be sufficient to survive numerous handling steps and produce a signal high enough to be detected by the chosen fluorescence detector.

In general, the transfected or transformed host cells are typically cultured by any conventional type of culture, such as batch, fed-batch, intensified fed-batch, or continuous. Suitable continuous cultures included repeated batch, chemostat, turbidostat or perfusion culture with product and cell retention or solely cell retention. However, for purposes of the invention, culturing is carried out in one or more single-use perfusion bioreactors, each of which can contain a volume of liquid culture medium of about 50 L to about 4000 L (e.g., 50 L, 60 L, 75 L, 100 L, 250 L, 500 L, 650 L, 750 L, 1000 L, 1250 L, 1500 L, 1750 L, 2000 L, 2250 L, 2500 L, 2750 L, 3000 L, 3250 L, 3500 L, 3750 L, or 4000 L), as desired. The number of single-use bioreactors employed to culture the cells is one, two, three, four, five, or six single-use perfusion bioreactors of the desired volume(s).

The host cells used to produce the protein of interest or “POI” (e.g., non-glycosylated or glycosylated proteins) in the invention can be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58: 44 (1979), Barnes et al., Anal. Biochem. 102: 255 (1980), U.S. Pat. Nos. 4,767,704; 4,657,866; 4,927,762; 4,560,655; or 5,122,469; WO90103430; WO 87/00195; or U.S. Pat. Re. No. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source, such that the physiological conditions of the cell in, or on, the medium promote expression of the protein of interest by the host cell; any other necessary supplements may also be included at appropriate concentrations that would be known to those skilled in the art.

The culture conditions, to be predetermined, such as temperature (for mammalian cells, typically, but not necessarily, about 37°±1° C.), pH (typically, but not necessarily, the cell culture medium is maintained within the range of about pH 6.5-7.5), oxygenation, and the like, will be apparent to the ordinarily skilled artisan. By “culturing at” or “maintaining at” a predetermined culture condition, is meant that the process control systems are set at a particular value for that condition, in other words the intended, volume, target temperature, pH, oxygenation level, or the like, maintained at predetermined set points for each parameter, within a narrow range (i.e., “narrow deadband”) most optimal for the cell line and protein product of interest. Clearly, there will be small variations of the temperature, pH, or other culture condition over time, and from location to location through the culture vessel (i.e., the bioreactor). (See, also, e.g., Oguchi et al., pH Condition in temperature shift cultivation enhances cell longevity and specific hMab productivity in CHO culture, Cytotechnology. 52(3):199-207 (2006); Al-Fageeh et al., The cold-shock response in cultured mammalian cells: Harnessing the response for the improvement of recombinant protein production, Biotechnol. Bioeng. 93:829-835 (2006); Marchant, R. J. et al., Metabolic rates, growth phase, and mRNA levels influence cell-specific antibody production levels from in vitro cultured mammalian cells at sub physiological temperatures, Mol. Biotechnol. 39:69-77 (2008)).

Digital control units and sensory monitors are available commercially or can be constructed by the skilled artisan. Alternative digital control units (DCU) control and monitor the cell culture process are available commercially, made by companies such as B. Braun, New Brunswick, Sartorius, or Thermo Fisher Scientific. Table 1A (below) lists some examples of digital control and sensory equipment that can be used to monitor cell culture conditions. Other on-line or off-line analyses can include off-gas measurements by mass spectrometry, in-depth determination of media composition (amino acids, vitamins, trace minerals) and expanded examination of cellular metabolites other than CO₂ and lactic acid.

TABLE 1A
Examples of commercially available cell
culture control and sensory equipment.

| Equipment | Description |
|---|---|
| Digital | Vendor- Specific (examples include Applikon, Wonderware |
| Control | (Aveva), DeltaV (Emerson), APACS (Siemens), Allen- |
| Unit or | Bradley (Rockwell), etc. |
| PLC Logic | |
| Controllers | |
| pH Probe | Hamilton EasyFerm Plus (potentiometric) |
| Dissolved | Hamilton VisiFerm (optical) or Broadley James |
| Oxygen | OxyProbe ® (polarographic) |
| Probe | |
| Gas flow | Solenoid-controlled gas flow consoles and/or mass flow |
| controller | controllers (MFCs); multiple vendors |
| Blood gas | Siemens RapidLab ® 248 or Siemens Rapidpoint ® 500 |
| analyzer | |
| Cell counter | Beckman Coulter Vi-Cell ® XR or Bioprofile ® CDV |
| | (Nova Biomedical Corp.) |
| Glucose, | YSI 2700 SELECT ™ Biochemistry analyzer (YSI Life |
| lactate and | Sciences) or Bioprofile ® Basic 2 (Nova Biomedical Corp.) |
| metabolite | |
| analyzer | |
| Osmometer | Advanced Instruments Model 2020 |

The culture medium can include a suitable amount of serum such a fetal bovine serum (FBS), or preferably, the host cells can be adapted for culture in serum-free medium. In some embodiments, the aqueous medium is liquid, such that the host cells are cultured in a cell suspension within the liquid medium. The host cells can be usefully grown in continuous (perfusion) cell culture systems, preferably that are designed for single-use.

In accordance with the invention, fresh culture medium is mixed contemporaneously from a plurality of concentrated component solutions and an aqueous diluent. Cell culture media are complex mixtures that contain a wide range of concentrations of each component as well as unique ratios of one component to another. The factor by which any cell culture medium formulation can be concentrated is limited by the solubility, stability, or filterability of its least soluble, least stable, or least filterable component. By dissolving components as chemically compatible subgroups, increased concentration factors can be achieved that would otherwise not be possible if all the components were dissolved together. For example, some components are more soluble at acidic pH while others are more soluble at alkaline pH. In this example, components that are soluble at acidic pH can be grouped together in one solution while components that are soluble at alkaline pH can be grouped together in another solution in such a way that when they are recombined they make a complete medium. In addition to or instead of pH grouping to achieve higher concentrations, one can utilize other solvents such as alcohol or dimethyl sulfoxide (DMSO); or, one can create stock solutions of individual components that have specialized solubility or storage requirements that necessitate their exclusion from other components until they are added to the bioreactor. The exact grouping of compatible components and their and maximum concentration for any given cell culture medium formulation is easily determined by those skilled in the art.

Typically, a viable cell density can be used from about 1.0×10⁶ up to about 2×10⁸ cells/mL, for example, in the range of 1.0×10⁶ to 2.0×10⁷ cells/mL, or in the range of about 4×10⁷ cells/mL to about 5×10⁷ cells/mL, or in the range of about 1×10⁸ cells/mL to about 2×10⁸ cells/mL. It is known that increasing the concentration of cells toward the higher end of the preferred ranges can improve volumetric productivity. Nevertheless, ranges of cell density including any of the above point values as lower or higher ends of a range are envisaged. The desired scale of the recombinant expression and cell culture will be dependent on the type of expression system and quantities of drug substance desired.

For purposes of the claimed invention, upon culturing the transfected or transformed host cells, the recombinant polypeptide or protein is directly secreted into the medium. Harvesting the recombinant protein involves separating it from particulate matter that can include host cells, cell aggregates, and/or lysed cell fragments, into a cell-free fraction that is free of host cells and cellular debris, i.e., a cell-free “permeate.” Such cells and cellular debris is removed from the conditioned medium, for example, by centrifugation and/or microfiltration. For example, to make the permeate, one can employ hollow fiber membranes (pore size 0.2 μm) or a series of filtration steps such as depth filtration, which can be configured on a mobile, interchangeable and/or single use and “filtration cart.”

Some embodiments of the invention include a first single-use surge vessel (SUSV1) adapted to receive volumes of permeate removed from the perfusion bioreactor(s); the volumes of permeate are cell free. These permeate volumes are automatically and fluidly fed from the one or more single-use perfusion bioreactor(s) into the SUV1. In some embodiments, there is an automated controller comprising detectors to measure the fluid volume in SUSV1, and a processor to vary the pump speeds of the first chromatography system to maintain a pre-set volume range in the SUSV1.

In some embodiments of the invention, the facility for practicing the process further comprises a hollow fiber membrane, a series of depth filters, or a filtration cart, to make the permeate cell free before it is automatically and fluidly fed to the SUSV1.

Protein Purification and Viral Inactivation

In general, the purification of proteins (e.g., recombinant or naturally occurring proteins) is usually accomplished by an optional series of chromatographic steps such as anion exchange chromatography, cation exchange chromatography, affinity chromatography (using Protein A or Protein G or Protein L as an affinity ligand or another different affinity ligand), hydrophobic interaction chromatography (HIC), hydroxy apatite chromatography, Reverse Phase HPLC, and size exclusion chromatography. The preceding are non-limiting examples of chromatographic modalities that can be included in any of the first chromatography system, the second chromatography system, and/or the third chromatography system. Each of the first, second, or third chromatography system(s) can be configured as needed for the protein of interest, preferably with one, two, three or more different chromatographic matrices (e.g., chromatography columns) fluidly linked in succession, and which, optionally, can be arranged in a mobile, interchangeable, or disposable, single-use unit, skid or “cart.” Further, the purification process may comprise one or more ultra-, nano- or diafiltration steps.

Other optional known techniques for protein purification such as ethanol precipitation, chromatofocusing, SDS-PAGE, and ammonium sulfate precipitation are also possible depending on the protein to be recovered.

In the inventive process for manufacturing a purified protein of interest (e.g., protein drug substance), the protein of interest (e.g., but not limited to, a protein drug) in the cell-free permeate is captured by one or more chromatographic capture steps of a first chromatography system that can partially purify and/or concentrate the protein, such as, but not limited to, Protein A or Protein G or Protein L affinity chromatography, or affinity chromatography employing a different affinity ligand covalently bound to a solid matrix. (See, e.g., Frank, M. B., “Antibody Binding to Protein A and Protein G beads” 5. In: Frank, M. B., ed., Molecular Biology Protocols. Oklahoma City (1997)). The first chromatography system can optionally include anion exchange chromatography (AEX), cation exchange chromatography (CEX), affinity chromatography (using Protein A or Protein G or Protein L as an affinity ligand or another particular target moiety), hydrophobic interaction chromatography (HIC), hydroxy apatite (HA) chromatography and size exclusion chromatography (SEC). In some embodiments involving a surge vessel upstream and fluidly connected to the first chromatography system, e.g., a first single-use surge vessel (SUSV1), there is an automated controller comprising detectors to measure the fluid volume in the surge vessel, e.g., the SUSV1, and a processor to vary the pump speeds of the first chromatography system to maintain a pre-set volume range in the surge vessel, (e.g., SUSV1). The volume of the SUSV1 is typically about 200 L, but can be set smaller or larger depending on the flow rates of the process and the desired residence time (which impacts the time frame allowed to react to process upsets). The operation of the first chromatography system collects or captures the protein of interest in a protein isolate fraction.

The first, second, and/or optional third chromatography system(s) are configured as needed for the protein of interest, preferably with one, two, three or more different chromatographic matrices (e.g., chromatography columns) fluidly linked in succession, and which, optionally, can be arranged in a mobile, interchangeable, or disposable, single-use unit, skid or “cart.”

In some embodiments of the invention, the second chromatography system comprises a single-use membrane adsorber (MA), such as, a surface-functionalized membrane. Such membrane adsorbers can involve anion-exchange groups for mAb polishing operations in negative mode, in which trace impurities are removed without binding the protein of interest (so-called “flow-through chromatography”). Examples, include, but are not limited to, Sartobind® Q or Sartobind STIC® (Sartorius Stedim Biotech), or Mustang® Q (Pall Life Sciences), or NatriFlo® HD-Q (Natrix Separations). Alternatively, membrane adsorbers can involve cation-exchange groups, e.g., Sartobind® S (Sartorius Stedim Biotec), or Mustang® S (Pall Life Sciences), or Natrix® HD-Sb (Natrix Separations). In some embodiments, membranes with other functional groups can be used to perform hydrophobic-interaction chromatography (HIC).

Embodiments of the inventive processes (and automated facilities) subsequently involve switching the protein isolate fraction obtained or collected from the first chromatography system, into a low pH or detergent viral inactivation system, and a neutralization system (i.e., if neutralization is needed subsequent to viral inactivation by low pH), to obtain a virally inactivated product pool comprising the protein of interest (e.g., but not limited to, a protein drug). However, optionally, before the protein isolate fraction is fluidly fed into the low pH or detergent viral inactivation system, the protein isolate fraction can be fluidly fed from the first chromatography system into, either:

(i) a second single-use surge vessel; or
(ii) at least two automatically switchable alternate single-use collection vessels (SUCV1 and SUCV2). The (i) single-use surge vessel, or (ii) the SUCV1 and SUCV2, are adapted to receive the protein isolate fraction from the first chromatography system and to fluidly feed the protein isolate fraction to the low pH or detergent viral inactivation system.

In an alternative embodiment, the low pH or detergent viral inactivation system and, if needed, the neutralization system (i.e., if neutralization is needed subsequent to viral inactivation by low pH), comprise:

(i) a (third) single-use surge vessel; or
(ii) at least two automatically switchable alternate single-use collection vessels (SUCV1 and SUCV2). The (i) single-use surge vessel, or (ii) the SUCV1 and SUCV2, comprised in the low pH or detergent viral inactivation system and, if needed, the neutralization system, are adapted to receive the protein isolate fraction from the first chromatography system.

The volumes of the SUSV2 and the SUCV1 and SUCV2 are typically about 100 L in volume, respectively, but depending on the frequency of further processing the pools, this can be made smaller or larger. For example, with elution pools of about 20-25 L, 50-L vessels were effectively used as SUCV1 and SUCV2. A neutralization system is needed to restore the isolated protein in solution to about neutral pH, after a low pH viral inactivation system has been used. The term “low pH” means a pH value of about pH 3.7 or lower, at which the protein isolate fraction is held (typically for at least 30-90 minutes) to inactivate any contaminating virus particles. (See, e.g., Chinniah, S et al., Characterization of operating parameters for XMuLV inactivation by low pH treatment, Biotechnol Prog. 32(1):89-97 (2016). If a detergent (e.g., Triton-X-100 and/or tri(n-butyl)phosphate (“TNBP”)) viral inactivation system is used, treatment of the protein isolate fraction by a neutralization system is not typically needed, unless lower than neutral pH conditions were also employed that would interfere with further effective purification or stable storage of the virally inactivated product pool. (See, e.g., Dichtelmüller et al., Effective virus inactivation and removal by steps of Biotest Pharmaceuticals, Results in Immunology 2:19-24 (2012); Ellgard et al., Evaluation of the virus clearance capacity and robustness of the manufacturing process for the recombinant factor VIII protein, turoctocog alfa IGIV production process, Protein Expression and Purification 129:94-100 (2017)).

The resulting virally inactivated product pool is subsequently introduced into the second chromatography system (in some embodiments, after being stored for at least 10 days or at least 20 days or at least 30 days) in a temperature controlled or chilled holding vessel (HV1) to obtain a purified product pool comprising the protein of interest. The second chromatography system is configured as needed for further purification of the protein of interest, preferably with one, two, three or more different chromatographic matrices (e.g., chromatography columns) fluidly linked in succession, and which, optionally, can be arranged in a mobile, interchangeable, or disposable, single-use unit, skid or “cart.”

Introducing the virally inactivated product pool into the second chromatography system is optionally controlled according to a coordinated schedule with respect to the culturing and viral inactivation steps. The coordinated schedule is calculated to maximize the efficient routing of virally inactivated product pool into the second chromatography system. This loading of the virally inactivated product pool into the second chromatography system according to the coordinated schedule is by automatic (continuous format) or batch-wise manual control (semi-continuous format). (See, also, Garcia, F A and Vandiver, M W, Throughput Optimization of Continuous Biopharmaceutical Manufacturing Facilities, PDA J Pharm Sci Technol 71(3):189-205 (2017)).

From the second chromatography system the resulting purified product pool comprising the protein of interest is switched fluidly into an optional third chromatography system and/or a viral filtration system to obtain a virus-free filtrate comprising the protein. Switching of the purified product pool into the optional chromatography system and/or viral filtration system is by automatic or manual control. The optional third chromatography system is configured, as needed for further purification of the protein of interest, preferably with one, two, three or more different chromatographic matrices (e.g., chromatography columns) fluidly linked in succession, and which, optionally, can be arranged in a mobile, interchangeable, or disposable, single-use unit, skid or “cart.” If a third chromatography system is not employed in the inventive process (or facility), then the purified product pool is switched and flows fluidly directly to the viral filtration system. Useful viral systems are commercially available, including single-use viral filtration systems.

The resulting virus-free filtrate is subsequently switched fluidly into an ultrafiltration/diafiltration system to obtain a composition comprising the purified protein of interest (e.g., a purified protein drug substance). Switching of the virus-free filtrate into the ultrafiltration/diafiltration system is by automatic or manual control.

Useful examples of ultrafiltration/diafiltration systems include ultrafiltration cassettes, such as, but not limited to, Pellicon® 3 Ultracel 30-kDa membranes (Millipore Sigma); Sartocon® ECO Hydrosart® 30-kDa regenerated cellulose membranes (Sartorius); Delta 30-kDa regenerated cellulose membranes (Pall Biotech), or the like.

At the end of the process, purified protein (e.g., a protein drug substance) can be stored in a sterile container, such as, but not limited to, single use sterile container (e.g., Celsius®-FFT system, Sartorius), a ready-to-use carboy, or can be processed directly to drug product.

In some embodiments of the inventive processes (and automated facilities) one or more of the first chromatography system, the second chromatography system, the third chromatography system, the low pH or detergent viral inactivation system, the neutralization system, the viral filtration system, or the ultrafiltration/diafiltration system, comprise single-use components. Employing single use components lends efficiency, safety, and lowers ultimate cost of practicing the inventive process.

Additionally, in scenarios where multiple single-use perfusion bioreactors are utilized in a facility for the production of a purified protein of interest (e.g., but not limited to, a purified protein drug substance), multiple operations performed with respect to each bioreactor can be performed concurrently. For example, while an ultrafiltration/diafiltration operation is taking place with respect to the virus-free filtrate produced from a first perfusion bioreactor, a chromatography operation can be performed with respect to a virally inactivated product pool produced by the viral inactivation system (and, if needed, the neutralization system) processing a protein isolate fraction received after processing by the first chromatography system of cell-free permeate derived from culturing in a second single-use perfusion bioreactor. In another example, while an ultrafiltration/diafiltration operation is taking place with respect to the virus-free filtrate ultimately produced by the inventive method from culturing in a first single-use perfusion bioreactor, a viral filtration operation can be performed with respect to a virally inactivated product pool ultimately produced by the inventive method from culturing in a second perfusion bioreactor. In additional embodiments, at least one chromatography process and/or viral filtration process performed on virus-free filtrate produced from a first perfusion bioreactor can take place during continuous chromatography capture or viral inactivation processes performed on cell-free permeate volumes produced by a second single-use bioreactor in accordance with the inventive process.

Purity of Water and other Ingredients. The water and all other ingredients that are used in the steps of the inventive process to express, purify and make formulations of the purified drug substance are preferably of a level of purity meeting the applicable legal or pharmacopoeial standards required for such pharmaceutical compositions and medicaments in the jurisdiction of interest, e.g., United States Pharmacopeia (USP), European Pharmacopeia, Japanese Pharmacopeia, or Chinese Pharmacopeia, etc. For example, according to the USP, Water for Injection is used as an excipient in the production of parenteral and other preparations where product endotoxin content must be controlled, and in other pharmaceutical applications, such as cleaning of certain equipment and parenteral product-contact components; and the minimum quality of source or feed water for the generation of Water for Injection is Drinking Water as defined by the U.S. Environmental Protection Agency (EPA), EU, Japan, or WHO.

Automation and Control Systems

Conventional production facility control systems are typically designed to control a preset configuration of equipment. In these scenarios, the logical and hardware couplings between pieces of equipment do not change. Thus, the identifiers and control operations that can be performed with respect to each piece of equipment are static. The implementations of production facility control systems described herein, with respect to the inventive automated facilities and processes for manufacturing a purified protein of interest (e.g., but not limited to, a protein drug substance), support variable configurations of equipment in a production line. In these situations, a piece of equipment can have different functionality, perform different operations, and/or be controlled using different sets of control commands and/or variables based on the location of the piece of equipment within a production line. Thus, the production lines and control systems described herein include software configurations and physical hardware that are different from conventional systems. The ability to configure a production line within an automated facility using a same group of control modules with different arrangements of pieces of equipment on the production line can be an implementation of so-called “FlexTrain” automation.

The implementations described herein can be performed by one or more systems that can automatically control the flow of material through each step of the process to produce a protein of interest, such as but not limited to, a protein drug substance. Alternatively, at least a portion of the control functions can be performed by operator intervention, and there may be circumstances (especially process disruptions) that may require operator intervention. The control functions can be performed using process data obtained from sensors coupled to various pieces of equipment used in the production of the purified protein of interest. The sensors can include temperature sensors, pH sensors, flow rate sensors, weight sensors (e.g., load cells), volume sensors (e.g., guided wave radar sensors), pressure sensors, timers, capacitance sensors, optical density sensors, or combinations thereof. The data generated by the sensors can be collected locally by the pieces of equipment. In certain embodiments, the pieces of equipment can forward the sensor data to a production facility control system. The production facility control system can collect data from sensors of a number of pieces of equipment being used to manufacture the purified protein of interest (e.g., a purified protein drug substance). The production facility control system can include one or more computing devices and/or one or more data stores that are in electronic communication with each other. At least a portion of the one or more computing devices and/or one or more data stores can be located in a same location, in some scenarios. Additionally, at least a portion of the one or more computing devices and/or the one or more data stores can be located remotely from the equipment included in a production facility. In this situation, at least a portion of the operations performed by the production facility control system can be implemented in a cloud computing architecture.

The data collected from the sensors can be stored in electronic data stores that can be referred to herein as “data historians.” In various implementations, a first data historian can collect and store data for at least a subset of the pieces of equipment operating in the purified protein production facility (e.g., for the production of a purified protein drug substance or other protein of interest). The first historian can store data for a period of time and then forward the data to a second data historian that is a repository for data collected regarding the operation of pieces of equipment coupled to the production facility control system. DeltaV historian and/or Pi historian are examples of commonly used redundant data historian systems in a commercial manufacturing plant for protein drug substances. In certain situations, the first data historian can then be reset and begin collecting and storing additional data from the purified protein production facility (e.g., for production of a purified protein drug substance) for an additional period of time. The production facility control system can also include one or more batch historians that collect and store data related to the operation of pieces of equipment included in the production facility for the production of particular batches of the purified protein of interest (e.g., but not limited to, a protein drug substance). The data historians can be accessed by the production facility control system and analyzed to determine parameters for the operation of pieces of equipment included under the controlof the production facility control system.

The production facility control system can analyze the data obtained from the sensors and determine operating conditions for one or more pieces of equipment. In some cases, the set points and acceptable operating parameters, and/or run recipe for the operation of a piece of equipment can be entered into the system by an operator. In other situations, the set points and acceptable operating parameters, and/or run recipe for the operation of a piece of equipment can be automatically sent to one or more pieces of equipment utilized in a purified protein production line (e.g., for the production of a purified protein drug substance). Alerts and alarm notifications can also be generated based on the sensor data. For example, in situations where sensor data indicates that an operating condition for a piece of equipment in a purified protein production line is outside of a threshold range, the system can trigger an alarm and send notification to an operator.

Various pieces of equipment used to produce the purified protein of interest (e.g., a purified protein drug substance) can include one or more communication interfaces that enable communications between the pieces of equipment and/or with the production facility control system. In some implementations the production facility control system can operate as a process automation system (PAS). The communication interfaces can include hardware devices, firmware devices, and/or software implemented systems that enable communication of data between pieces of equipment used in a purified protein production line and/or with the production facility control system. The communication interfaces can enable communication of data over a number of networks, such as local area wired networks, local area wireless networks, wide area wireless networks, and/or wide area wired networks. In particular examples, the communication interfaces can include Ethernet network communication interfaces, Internet Protocol network communication interfaces, Institute of Electrical and Electronics Engineers (IEEE) 802.11 wireless network communication interfaces, Bluetooth communication interfaces, or combinations thereof.

The pieces of equipment used to produce the purified protein of interest (e.g., but not limited to, a protein drug substance) can include one or more processors and one or more memory devices. The one or more processors can be central processing units, such as standard programmable processors that perform arithmetic and logical operations necessary for the operation of computing systems. The one or more memory devices can include volatile and nonvolatile memory and/or removable and non-removable media implemented in any type of technology for storage of information, such as computer-readable instructions, data structures, program modules, or other data. Such computer-readable storage media can include, but is not limited to, RAM, ROM, EEPROM, flash memory or other memory technology, CD-ROM, digital versatile disks (DVD) or other optical storage, magnetic cassettes, magnetic tape, solid state storage, magnetic disk storage, RAID storage systems, storage arrays, network attached storage, storage area networks, cloud storage, removable storage media, or any other medium that can be used to store the desired information and that can be accessed by the production facility control system or by the individual pieces of equipment included in a purified protein production line.

In accordance with the inventive automated facilities and processes for manufacturing a purified protein of interest (e.g., but not limited to, a protein drug substance), at least a portion of the pieces of equipment included in the purified protein production line, and the production facility control system can store one or more modules that can be executed to control the operation of the pieces of equipment included in the purified protein production line. The modules can include computer-readable instructions that can be executed to cause the pieces of equipment included in the purified protein production line to take one or more actions. The modules can be part of a framework that enables the pieces of equipment included in the purified protein production line to produce the purified protein (e.g., the purified protein drug substance) in a continuous or semi-continuous manner. The actions performed by various pieces of equipment included in the purified protein production line can be related to start up processes, hold processes, shutdown processes, feed processes, or end of production processes.

In particular embodiments, the control systems described herein can be used to control production lines that have flexible configurations. That is, the control systems described herein can accommodate multiple configurations that utilize portable equipment that can be coupled to other components of the production line. In various embodiments, the production line can include one or more skids that include original manufacturer's equipment, such as a single-use bioreactor system, a perfusion system, or a continuous chromatography system. The skids can also include flow control devices, such as pumps. Additionally, the skids can include one or more communication interfaces, also referred to herein as “drops,” that enable the physical coupling of portable pieces of equipment to the skid. The physical coupling between the portable pieces of equipment and the skid can be achieved using electrical cabling. The electrical cabling can be configured to enable ethernet communications. In certain examples, the electrical cabling can be Recommended Standard 232 (RS-232) cabling.

The portable pieces of equipment can include or otherwise be coupled to a network gateway hardware device that enables communication between the respective portable pieces of equipment and the production facility control system. The network gateway hardware device for each portable piece of equipment can be coupled to a communication interface of a respective skid. In addition, at least some of the skids can be logically configured to be coupled to various pieces of portable equipment. In this way, the pieces of portable equipment can be physical connected to a particular skid based on the configuration of a particular production line and the skids can be configured to operate in different configurations based on the different pieces of equipment coupled to the skid.

Additionally, the portable pieces of equipment can be coupled to at least one information communication and/or storage device, such as a dongle. The information communication and/or storage device can store information that is provided to the respective piece of equipment to which it is coupled that enables control of the respective piece of equipment via the production facility control system. The information communication and/or storage device can store information that includes one or more identifiers of a respective piece of equipment, one or more functions of the respective piece of equipment, one or more control signals corresponding to the respective piece of equipment, one or more status flags related to the respective piece of equipment, or combinations thereof. In some examples, the data stored by the information communication and/or storage device can be based at least partly on the functions, or a type, of the respective piece of equipment. In situations where a portable piece of equipment is placed in a different location along a production line and/or has a different function, the information communication and/or storage device of the portable piece of equipment can be switched to an additional information communication and/or storage device that indicates a different function and a different identifier for the portable piece of equipment.

Further, the control systems described herein can include an additional logical layer that can be used on top of conventional control software and systems. In particular implementations, the control systems described herein can include an additional abstraction layer that enables the assignment, also referred to as “binding,” of the portable pieces of equipment to various identifiers, tags, operating conditions, and flags that correspond to a specified set of functions for a specific piece of equipment at a particular location along the production line. In this way, a piece of equipment is not logically represented in the control system until the location and function of the piece of equipment is known. Thus, portable pieces of equipment can be coupled with skids in a variety of combinations without having to change the underlying control software that is being utilized to control the components of the skids and also control the portable pieces of equipment.

In illustrative examples, a production line in accordance with the inventive automated facility for manufacturing a purified protein of interest (e.g., but not limited to, a purified protein drug substance) can include a first skid that includes a single use bioreactor system, a second skid that includes a perfusion system, and a third skid that includes a continuous first chromatography system. The skids can be configured to couple to multiple portable pieces of portable equipment. For example, the skids can include interfaces and physical hardware to couple to portable mix tanks, filter banks, storage containers, surge vessels, holding vessels, diverter valve systems (for switching automatically switchable alternate dual flow path or multi-flow path unit operations, e.g., SUCV1 and SUCV2), and/or other flow control devices. Some of the mix tanks (or interchangeably, “mixing vessels”) or storage containers can serve as feed tanks or collection vessels, which can function as surge vessels in a continuous or semi-continuous format manufacturing process, or function as holding vessels in a batch mode format manufacturing process.

After coupling a piece of portable equipment to a skid, the piece of portable equipment can be registered with the production facility control system. The piece of portable equipment can have a unique address that the piece of portable equipment can communicate to the production facility control system. The unique address can indicate a type of the piece of portable equipment and a unit identifier to the production facility control system. A dongle coupled to the piece of portable equipment can store an additional identifier that corresponds to a location of the skid to which the portable piece of equipment is coupled and one or more functional roles of the portable piece of equipment. For example, a mix tank can be identified as a feed tank, or a collection tank based on the location of the portable piece of equipment and the logical association of the drop to which the portable piece of equipment is coupled. In another example, a filter bank can be identified as a viral filtration device in a first configuration of a production line and then identified as a diafiltration device in a second configuration of a production line. In these situations, a first dongle can be coupled to the filter bank in the first configuration of the production line and a second dongle can be coupled to the filter bank in the second configuration of the production line. Additionally, the type of filter used in the filter bank can be changed when the filter bank is used in different locations of a production line.

In response to obtaining the information from the portable piece of equipment after being coupled to the skid, the production facility control system can determine the location and functions of the portable piece of equipment and assign the corresponding control templates to the portable piece of equipment. For example, in situations where a mix tank is functioning as a collection tank, the production facility control system can assign a first set of tags, flags, identifiers, and set points to the mix tank and in situations where a mix tank is functioning as a feed tank, the production facility control system can assign a second set of tags, flags, identifiers, and set points to the mix tank. The production facility control system can then assign a particular set of control modules to the portable piece of equipment based on the information obtained from the portable piece of equipment after being coupled to the skid.

In various embodiments, pieces of equipment that are not considered portable, such as large collection tanks can also be coupled to the skid. In these scenarios, the non-portable pieces of equipment may not include the hardware and/or communication and storage devices that enable dynamic configuration of the non-portable piece of equipment with respect to the production facility control system. If the non-portable piece of equipment is not configured for a dynamic configuration, an operator of the production facility control system can manually establish the template and/or control module used to control the operation of the non-portable piece of equipment.

In addition to the control of the pieces of equipment included in a production line, the production facility control system can also track the decay rate of a batch during production of a purified protein of interest (e.g., but not limited to, a purified protein drug substance). The “decay rate” is a period of time in which materials used for the production of sub-lots can be identified and tracked. For example, the materials used (e.g., buffers, cell culture medium, etc.) in a resulting chromatography step eluate pool collection, of which there may be many, can be identified and tracked in a dynamic fashion by way of the “decay rate.” In a continuous batch production process, the production facility control system can estimate the decay rate for the purified protein production process. In various implementations, the production facility control system can assign batch identifiers to certain portions of the production of the batch and initiate a decay monitor until the current batch identifier is changed to a new batch identifier and a new decay monitor is implemented for the new batch identifier.

In an illustrative example, the production facility control system can determine that a filter bank is coupled between a perfusion bioreactor and a first chromatography system based on information obtained from a dongle coupled to the filter bank. In these situations, the filter bank can operate as a depth filter. The production facility control system can identify one or more control modules, flags, and/or status identifiers for a depth filter and execute the one or more control modules while the filter bank is being used in a production line. The production facility control system can monitor pressure within the filter assemblies of the filter bank based on pressure values obtained from pressure sensors included in the filter assemblies. The production facility control system can determine that the pressure within a first filter assembly through which material is flowing has reached at least a threshold level. The threshold level of pressure can indicate that a filter included in the first assembly needs to be replaced due to a decrease in the amount of material that can be processed by the filter. The production facility control system can then send a signal to control a diverter valve coupled to the filter bank to cause the material to flow through a second filter assembly of the filter bank. The filter included in first filter assembly can then be replaced.

By way of further illustration, the following embodiments of the present invention are enumerated:

Embodiment 1: A process for manufacturing a purified protein of interest, the process comprising the step of:

(a) culturing mammalian cells in one or more single-use perfusion bioreactors comprising a liquid culture medium under conditions that allow the cells to secrete the protein into the liquid culture medium for a production cultivation period of at least 10 days, wherein, periodically or continuously, during the production cultivation period, fresh sterile liquid culture medium is added into the one or more perfusion bioreactors, to maintain a constant culture volume in each of the perfusion bioreactor(s), in direct relation to volumes of the culture that are continuously or periodically removed from each of the perfusion bioreactor(s) as volumes of permeate or cell bleed, and wherein the removed volumes of permeate are automatically and fluidly fed from the one or more single-use perfusion bioreactor(s) into a single-use surge vessel and thence into a first chromatography system, whereby the protein is collected in a protein isolate fraction.

Embodiment 2: A process for manufacturing a purified protein of interest, the process comprising the step of:

(a) culturing mammalian cells in one or more single-use perfusion bioreactors comprising a liquid culture medium under conditions that allow the cells to secrete the protein into the liquid culture medium for a production cultivation period of at least 10 days, wherein, periodically or continuously, during the production cultivation period, fresh sterile liquid culture medium is added into the one or more perfusion bioreactors, being mixed contemporaneously from a plurality of different concentrated medium component solutions and an aqueous diluent, to maintain a constant culture volume in each of the perfusion bioreactor(s), in direct relation to volumes of the culture that are continuously or periodically removed from each of the perfusion bioreactor(s) as volumes of permeate or cell bleed, and wherein the removed volumes of permeate are automatically and fluidly fed from the one or more single-use perfusion bioreactor(s) into a single-use surge vessel and thence into a first chromatography system, whereby the protein is collected in a protein isolate fraction.

Embodiment 3: The process of Embodiments 1-2, further comprising the step of:

(b) switching the protein isolate fraction into a low pH or detergent viral inactivation system and, if needed, a neutralization system, to obtain a virally inactivated product pool comprising the protein.

Embodiment 4: The process of any of Embodiments 1-3, further comprising the steps of:

(c) introducing the virally inactivated product pool into a second chromatography system to obtain a purified product pool comprising the protein;

(d) switching the purified product pool comprising the protein into an optional third chromatography system and/or a viral filtration system to obtain a virus-free filtrate comprising the protein; and

(e) switching the virus-free filtrate into an ultrafiltration/diafiltration system to obtain a composition comprising the purified protein of interest.

Embodiment 5: The process of any of Embodiments 1-4, wherein the protein of interest is a recombinant protein.

Embodiment 6: The process of any of Embodiments 1-5, wherein the protein of interest is a therapeutic protein (or other medically useful protein).

Embodiment 7: The process of any of Embodiments 1-6, wherein one or more of the first chromatography system, the second chromatography system, the third chromatography system, the low pH or detergent viral inactivation system, the neutralization system, the viral filtration system, or the ultrafiltration/diafiltration system, comprise a single-use component(s).

Embodiment 8: The process of any of Embodiments 1-7, wherein the mammalian cells are cultured in two, three, four, five, or six single-use perfusion bioreactors.

Embodiment 9: The process of any of Embodiments 1-8, wherein the one or more single-use bioreactor(s) can contain a volume of liquid culture medium about 50 L to about 4000 L.

Embodiment 10: The process of any of Embodiments 2-9, wherein the fresh sterile liquid culture medium is added to the one or more perfusion bioreactors, by injecting the plurality of different concentrated medium component solutions at fixed ratios relative to one another, directly into the perfusion bioreactor(s), while an aqueous diluent is also added at varied ratio(s) relative to the plurality of different concentrated component solutions, to maintain a constant culture volume in each perfusion bioreactor(s).

Embodiment 11: The process of any of Embodiments 2-9, wherein the fresh sterile liquid culture medium is added to the one or more perfusion bioreactors, by injecting the plurality of different concentrated medium component solutions and the aqueous diluent at fixed ratios relative to one another, directly into the perfusion bioreactor(s), to maintain a constant culture volume in each perfusion bioreactor(s).

Embodiment 12: The process of any of Embodiments 2-9, wherein the fresh sterile liquid culture medium is added to the one or more perfusion bioreactors, by injecting the plurality of different concentrated medium component solutions and the aqueous diluent, at fixed ratios relative to one another, into a mixing chamber wherein fresh sterile liquid culture medium is mixed contemporaneously before being added to each perfusion bioreactor(s) to maintain a constant culture volume.

Embodiment 13: The process of any of Embodiments 1-12, wherein an automated controller comprising a detector is used to measure the fluid volume in the single-use surge vessel, and a processor varies the pump speeds of the first chromatography system to maintain a pre-set volume range in the single-use surge vessel.

Embodiment 14: The process of any of Embodiments 3-13, wherein one or more of steps (b), (c), (d), or (e) is performed automatically and fluidly in an uninterrupted flow from the previous step, and wherein a surge vessel is employed between one or more steps, and a processor varies the pump speed in a subsequent step to regulate the pre-set volume range of the surge vessel preceding the subsequent step.

Embodiment 15: The process of any of Embodiments 3-14, wherein in-line or in-vessel conditioning of pH and/or conductivity load, is performed between the one or more of steps (b), (c), (d), or (e).

Embodiment 16: The process of any of Embodiments 1-15, wherein:

(i) a process automation system is in electronic communication with at least the one or more single-use perfusion bioreactors, the single-use surge vessel, and the first chromatography system;

(ii) the process automation system stores a first set of control modules to control operation of at least one single-use perfusion bioreactor of the one or more single-use perfusion bioreactors;

(iii) the process automation system stores a second set of control modules to control operation of feed tanks;

(iv) the process automation system stores a third set of control modules to control operation of collection tanks; and

(v) the at least one single-use perfusion bioreactor is logically configured to be coupled to one or more feed tanks, one or more collection tanks, or a filter bank.

Embodiment 17: The process of Embodiment 16, wherein the at least one single-use perfusion bioreactor is disposed on a skid and the skid includes a plurality of communication interfaces to electronically couple the at least one single-use perfusion bioreactor to a plurality of pieces of portable equipment.

Embodiment 18: The process of Embodiment 17, further comprising:

(vi) determining, by the process automation system, that the single-use surge vessel has been coupled to a communication interface of the plurality of communication interfaces based on data received via the communication interface, the data indicating an identifier of the single-use surge vessel and a function of the single-use surge vessel; and

(vii) determining, based at least partly on the identifier and the function of the single-use surge vessel, that the single-use surge vessel is a collection tank and that the third set of control modules is to control operation of the single-use surge vessel.

Embodiment 19: The process of Embodiment 18, further comprising:

(viii) determining, by the process automation system, that a mixing vessel has been coupled to an additional communication interface of the plurality of communication interfaces based on additional data received via the additional communication interface, the additional data indicating an additional identifier of the mixing vessel and an additional function of the mixing vessel; and

(ix) determining, based at least partly on the additional identifier and the additional function of the mixing vessel, that the mixing vessel is a feed tank and that the second set of control modules is to control operation of the mixing vessel.

Embodiment 20: The process of any of Embodiments 18-19, wherein the identifier of the single-use surge vessel and the function of the single-use surge vessel are stored on a dongle coupled to the single-use surge vessel.

Embodiment 21: The process of any of Embodiments 1-20, wherein the production cultivation period is at least 20 days.

Embodiment 22: An automated facility for manufacturing a purified protein of interest, the facility comprising:

(a) one or more single-use perfusion bioreactors capable of containing a liquid culture medium under conditions that allow cultured cells to secrete the protein into the liquid culture medium for a production cultivation period of at least 10 days; wherein the single-use perfusion bioreactor(s) are adapted to receive fresh sterile liquid culture medium fluidly into each of the perfusion bioreactor(s) in direct relation to volumes of conditioned culture medium that are continuously or periodically removed from each of the perfusion bioreactor(s) as volumes of permeate or cell bleed during the production cultivation period;

(b) a first single-use surge vessel (SUSV1) into which said removed volumes of permeate are automatically and fluidly fed from the one or more single-use perfusion bioreactor(s); and

(c) a first chromatography system, adapted to automatically and fluidly receive cell-free permeate from the SUSV1, whereby the protein is captured in a protein isolate fraction; and

wherein the automated facility is controlled by a process automation system (PAS).

Embodiment 23: The automated facility of Embodiment 22, further comprising: a plurality of reservoirs, each adapted for containing a concentrated medium component solution or aqueous diluent, the plurality of reservoirs being fluidly connected to the perfusion bioreactor(s) directly, or indirectly via an optional mixing vessel adapted for receiving from the plurality of reservoirs the concentrated culture medium component solutions and aqueous diluent at predetermined ratios and contemporaneously mixing them, the optional mixing vessel being fluidly connected directly to the perfusion bioreactor(s).

Embodiment 24: The automated facility of Embodiment 22-23, further comprising:

(d) a low pH or detergent viral inactivation system and, if needed, a neutralization system, adapted to automatically and fluidly receive the protein isolate fraction from the first chromatography system, whereby a virally inactivated product pool comprising the protein is obtained; and

(e) a holding vessel or a second single-use surge vessel, adapted for receiving the virally inactivated product pool.

Embodiment 25: The automated facility of Embodiment 22-24, further comprising:

(f) a second chromatography system adapted to fluidly receive from the holding vessel or the second single-use surge vessel the virally inactivated product pool, whereby a purified product pool comprising the protein is obtained;

(g) an optional third chromatography system and/or a viral filtration system adapted to fluidly receive the purified product pool comprising the protein from the second chromatography system, whereby a virus-free filtrate comprising the protein is obtained; and

(h) an ultrafiltration/diafiltration system adapted to fluidly receive the virus-free filtrate from the second chromatography system or from the third chromatography system and/or the viral filtration system, whereby the purified protein of interest is obtained.

Embodiment 26: The automated facility of any of Embodiments 22-25, wherein one or more single-use perfusion bioreactors can contain a volume of liquid culture medium of about 50 L to about 4000 L.

Embodiment 27: The automated facility of any of Embodiments 22-26, further comprising an automated controller comprising a detector to measure the fluid volume in SUSV1, and a processor to vary the pump speeds of the first chromatography system to maintain a pre-set volume range in SUSV1.

Embodiment 28: The automated facility of any of Embodiments 24-27, wherein one or more of the first chromatography system, the second chromatography system, the third chromatography system, the low pH or detergent viral inactivation system, the neutralization system, the viral filtration system, or the ultrafiltration/diafiltration system, comprise a single-use component(s).

Embodiment 29: The automated facility of any of Embodiments 24-28, further comprising, and fluidly connected directly downstream from the first chromatography system:

(i) a second single-use surge vessel; or

(ii) at least two automatically switchable alternate single-use collection vessels (SUCV1 and SUCV2) adapted for receiving the protein isolate fraction;

wherein (i) and (ii) are adapted to receive the protein isolate fraction from the first chromatography system and to fluidly feed the protein isolate fraction to the low pH or detergent viral inactivation system.

Embodiment 30: The automated facility of any of Embodiments 24-28, wherein the low pH or detergent viral inactivation system and, if needed, the neutralization system, comprises:

(i) a second single-use surge vessel adapted for receiving the protein isolate fraction; or

(ii) at least two automatically switchable alternate single-use collection vessels (SUCV1 and SUCV2) adapted for receiving the protein isolate fraction;

wherein viral inactivation, and if needed neutralization, is conducted within the second single-use surge vessel, or within SUCV1 and SUCV2.

Embodiment 31: The automated facility of any of Embodiments 22-30, further comprising a hollow fiber membrane, a series of depth filters, or a filtration cart, before the permeate is automatically and fluidly fed to the SUSV1.

Embodiment 32: The automated facility of any of Embodiments 22-31, comprising in (e) a single-use surge vessel adapted for receiving the virally inactivated product pool.

Embodiment 33: The automated facility of any of Embodiments 22-32, further comprising a heat exchanger upstream of the SUSV1.

Embodiment 34: The automated facility of any of Embodiments 22-33, further comprising a filtration system upstream of the SUSV1.

Embodiment 35: The automated facility of any of Embodiments 24-34, wherein one or more of:

(i) the second chromatography system;

(ii) the optional third chromatography system;

(iii) the viral filtration system; and

(iv) the ultrafiltration/diafiltration system,

is automatically and fluidly connected to the previous system, and wherein a surge vessel is optionally employed to regulate the uninterrupted flow of material between the connected systems.

Embodiment 36: The automated facility of any of Embodiments 22-35, wherein:

at least the one or more single-use perfusion bioreactors, SUSV1, the first chromatography system, the low pH or detergent viral inactivation system, the holding vessel or single-use surge vessel, the second chromatography system, the optional third chromatography system and/or the viral filtration system, and the ultrafiltration/diafiltration system comprise first pieces of equipment that are arranged in a first configuration of a production line for the purified protein of interest; and

a first plurality of control modules are implemented to control operation of the first pieces of equipment.

Embodiment 37: The automated facility of Embodiment 36, wherein:

second pieces of equipment are arranged in a second configuration of a production line for an additional purified protein of interest, the second configuration of the production line including at least the one or more single-use perfusion bioreactors, the first chromatography system, the low pH or detergent viral inactivation system, the second chromatography system, the ultrafiltration/diafiltration system, and a plurality of mixing vessels;

the second configuration of the production line being different from the first configuration of the production line;

a second plurality of control modules are implemented to control operation of the second pieces of equipment;

at least one mixing vessel of the plurality of mixing vessels is included in both the first configuration and the second configuration; and

the at least one mixing vessel has a first function in the first configuration and a second function in the second configuration, the second function being different from the first function.

Embodiment 38: The automated facility of any of Embodiments 22-37, further comprising a portable filter bank, the portable filter bank including a plurality of filter assemblies, wherein:

a first filter assembly of the plurality of filter assemblies includes a first filter and a second filter assembly of the plurality of filter assemblies includes a second filter; and

a production facility control system:

monitors a pressure within the first filter assembly as material flows through the first filter assembly;

determines that the pressure within the first filter assembly is at least a threshold value; and

sends a signal to cause a diverter valve coupled to the first filter assembly and the second filter assembly to operate to cause the material to flow into second filter assembly.

Embodiment 39: The automated facility of Embodiment 38, wherein, during a first period of time, the filter bank is coupled between the first single-use surge vessel (SUSV1) and one or more single-use perfusion bioreactors, the material includes the permeate, and the filter bank is coupled to a first dongle indicating a first identifier for the filter bank and a first function for the filter bank.

Embodiment 40: The automated facility of Embodiment 39, wherein, during a second period of time, the filter bank is included in the low pH or detergent viral inactivation system, the material is the virus free filtrate, and the filter bank is coupled to a second dongle indicating a second identifier of the filter bank and a second function for the filter bank.

Embodiment 41: The automated facility of any of Embodiments 22-40, wherein the one or more single-use perfusion bioreactors is capable of containing a liquid culture medium under conditions that allow the cultured cells to secrete the protein into the medium for a production cultivation period of at least 20 days.

Embodiment 42: The process of any of Embodiments 1-21 or the automated facility of any of Embodiments 22-41, wherein the protein of interest is a recombinant protein and/or a therapeutic protein.

Embodiment 43: A process for manufacturing a purified protein drug substance comprising a protein of interest, the process comprising the steps of:

(a) culturing mammalian cells in one or more single-use perfusion bioreactors comprising a liquid culture medium under conditions that allow the cells to secrete the protein into the medium for a production cultivation period of at least 10 days, wherein, periodically or continuously, during the production cultivation period, fresh sterile liquid culture medium is added into the one or more perfusion bioreactors, to maintain a constant culture volume in each of the perfusion bioreactor(s), in direct relation to volumes of the culture that are continuously or periodically removed from each of the perfusion bioreactor(s) as volumes of permeate or cell bleed, and wherein the removed volumes of permeate are automatically and fluidly fed from the one or more single-use perfusion bioreactor(s) into a single-use surge vessel and thence into a first chromatography system, whereby the protein is collected in a protein isolate fraction;

(b) switching the protein isolate fraction into a low pH or detergent viral inactivation system and, if needed, a neutralization system, to obtain a virally inactivated product pool comprising the protein;

(c) introducing the virally inactivated product pool into a second chromatography system to obtain a purified product pool comprising the protein;

(d) switching the purified product pool comprising the protein into an optional third chromatography system and/or a viral filtration system to obtain a virus-free filtrate comprising the protein; and

(e) switching the virus-free filtrate into an ultrafiltration/diafiltration system to obtain the purified protein drug substance comprising the protein of interest.

Embodiment 44: The process of any of Embodiments 42-43, wherein the fresh sterile liquid culture medium is mixed contemporaneously from a plurality of different concentrated medium component solutions and an aqueous diluent, before being added into the one or more perfusion bioreactors to maintain a constant culture volume in each of the perfusion bioreactor(s).

Embodiment 45: An automated facility for manufacturing a purified protein drug substance, the facility comprising:

(a) one or more single-use perfusion bioreactors capable of containing a liquid culture medium under conditions that allow cultured mammalian cells to secrete a protein of interest into the medium for a production cultivation period of at least 10 days; wherein the single-use perfusion bioreactor(s) are adapted to receive fresh sterile liquid culture medium fluidly into each of the perfusion bioreactor(s) in direct relation to volumes of conditioned culture medium that are continuously or periodically removed from each of the perfusion bioreactor(s) as volumes of permeate or cell bleed during the production cultivation period;

(b) a first single-use surge vessel (SUSV1) into which said removed volumes of permeate are automatically and fluidly fed from the one or more single-use perfusion bioreactor(s);

(c) a first chromatography system, adapted to automatically and fluidly receive permeate from the SUSV1, whereby the protein is captured in a protein isolate fraction;

(d) a low pH or detergent viral inactivation system and, if needed, a neutralization system, adapted to automatically and fluidly receive the protein isolate fraction from the first chromatography system, whereby a virally inactivated product pool comprising the protein is obtained;

(e) a holding vessel or a single-use surge vessel, adapted for receiving the virally inactivated product pool;

(f) a second chromatography system adapted to fluidly receive from the holding vessel or single-use surge vessel the virally inactivated product pool, whereby a purified product pool comprising the protein is obtained;

(g) an optional third chromatography system and/or a viral filtration system adapted to fluidly receive the purified product pool comprising the protein from the second chromatography system, whereby a virus-free filtrate comprising the protein is obtained; and

(h) an ultrafiltration/diafiltration system adapted to fluidly receive the virus-free filtrate from the second chromatography system or from the third chromatography system and/or the viral filtration system, whereby the purified protein drug substance is obtained; and

wherein the automated facility is controlled by a process automation system (PAS).

Embodiment 46: The automated facility of Embodiment 45, wherein a plurality of reservoirs, each adapted for containing a concentrated medium component solution or aqueous diluent, are fluidly connected to the perfusion bioreactor(s) directly, or indirectly via an optional mixing vessel adapted for receiving from the plurality of reservoirs the concentrated culture medium component solutions and aqueous diluent at predetermined ratios and contemporaneously mixing them, the optional mixing vessel being fluidly connected directly to the perfusion bioreactor(s).

Embodiment 47: The process of any of Embodiments 42-44 or the automated facility of any of Embodiments 45-46, wherein the protein of interest is a recombinant protein and/or a therapeutic protein.

Embodiment 48: The automated facility of any of Embodiments 45-46, wherein the protein of interest is a recombinant protein and/or a therapeutic protein.

Embodiment 49: The automated facility of any of Embodiments 22-42 or any of Embodiments 45-46 or any of Embodiments 48-49, wherein the facility is configured for operation in a continuous format.

Embodiment 50: The process of any of Embodiments 1-21 or any of Embodiments 42-44, wherein the process is conducted in a continuous format.

Embodiment 51: The process of any of Embodiments 1-21 or any of Embodiments 42-44, wherein the first chromatography system is sanitized with a chemical sanitant solution comprising peracetic acid before use.

Embodiment 52: The process of any of Embodiment 4 or Embodiments 43-44, wherein the ultrafiltration/diafiltration system comprises a single pass tangential flow filtration (SPTFF), and the operating pressure of the SPTFF is controlled in a range of about 0.25 psi to about 60 psi.

Embodiment 53: The process of any of Embodiment 4 or Embodiments 43-44, wherein the ultrafiltration/diafiltration system comprises inline depth filtration (ILDF), and the operating pressure of the ILDF is controlled in a range of about 0.25 psi to about 60 psi.

Embodiment 54: The process of any of Embodiments 52-53, wherein the operating pressure of the SPTFF and/or the ILDF is controlled in a range selected from the group consisting of about 0.25 psi to about 45 psi, about 0.25 psi to about 30 psi, about 0.25 psi to about 15 psi, and about 0.25 psi to about 5 psi.

The following working examples are illustrative and not to be construed in any way as limiting the scope of the invention.

### EXAMPLES

### Example 1. Demonstration of Continuous Perfusion Culture and Protein Product Capture Chromatography for Extended Production Cultivation Period

Materials and Methods

A set of three engineering runs were performed at 500-L bioreactor scale to demonstrate the inventive process for manufacturing a purified protein (in this example, a recombinant therapeutic protein drug substance), encompassing the use of contemporaneously mixed concentrated medium components. Corresponding 2-L satellite bioreactors were operated to generate data using 1× delivered medium at small-scale by way of comparison.

Protein of interest, host cells, culture medium. The recombinant therapeutic protein of interest that was produced and isolated for demonstration purposes was an IgG1κ isotype monoclonal antibody, produced by a recombinant CHO-K1 cell line, cultured in a chemically defined cell culture medium.

Perfusion bioreactor and first chromatography system. A perfusion bioreactor employed a Xcellerex® XDR 500-L single-use (stirred-tank) bioreactor (SUB; GE Healthcare Life Sciences), which was connected to a Spectrum Krosflo® KPS-600 perfusion system (Repligen Corporation). The Xcellerex® XDR 500-L SUB had blend time(s) from 30-55 seconds at agitation rates of 95-150 rpm. Shorter blend times are also possible by increasing agitation; however, these were not characterized. The perfusion system was installed with a hollow fiber filter 0.2 μm pore size that retains cells on the retentate side while allowing high product passage on the permeate side. During the initial startup of the perfusion culture, the permeate fluid waste was sent directly to drain via a single-use air break assembly. When the Protein A chromatography product capture operation was initiated, the permeate stream was diverted to a filter cart, and the single-use air break assembly was stored in Minncare Sterilant peracetic acid solution (Mar Cor Purification). The filter cart, with DeltaV automation, included a primary and backup sterilizing grade filter (Express® SHC, 0.2 μm; MilliporeSigma) acting as a guard filter for the primary capture chromatography columns. Other 0.2-μm filters that can be used in the filter cart include Sartopore® 2 (Sartorius), Pall Fluorodyne® EX grade EDF filters, or the like. An optional heat exchanger with single-use bag assembly can effectively control the temperature of the chromatography load material, but was not used for these runs. However, in other embodiments of the process and automated facility, a single-use heat exchanger is used (Thermo Scientific™ DHX™ Heat Exchanger with a Thermo Scientific™ ThermoFlex™ Recirculating Chiller, and Thermo Scientific™ DHX™ Bag Assembly).

A 200-L portable mixer served as a single-use surge vessel (SUSV), which was employed as a pressure break between the pumps of the perfusion system and the first chromatography system and to manage discrepant flow rates between these two fluidly connected and continuous unit operations. The multi-column capture chromatography system employed a continuous single-use, multi-column chromatography system (Cadence™ BioSMB® PD, hereinafter abbreviated, “BioSMB”; Pall Life Sciences), which is a multi-column continuous chromatography (MCC) system designed with a fully disposable flow path, and for this process operates three 14 cm-D×5 cm-H acrylic columns packed with Protein A resin. The elution outlet of the BioSMB system was connected to two alternating elution pool collection vessels to allow simultaneous collection of the elution pool while further processing the low pH viral inactivation and neutralization step. A schematic partial process flow diagram of the system is shown in FIG. 1B. In FIG. 1B, a filter cart sits upstream to the SUSV (labeled “Non-Batch Unit (B1)” or “200 L portable mixer” in FIG. 1B) and contains a 0.2 μm filter (e.g., Millipore Express SHC; Sartorius Sartopore 2; Pall Fluorodyne EDF) to filter the perfusion permeate before it is loaded on to a Protein A affinity chromatography column in the first chromatography system; the filter acts as a guard filter, protecting the first chromatography system from particulates. Also shown in FIG. 1B is an optional heat exchanger of single-use plate and frame design, which can be used to chill the warmer perfusion permeate fluid to room temperature or to a different desired target temperature for the SUSV and first chromatography system.

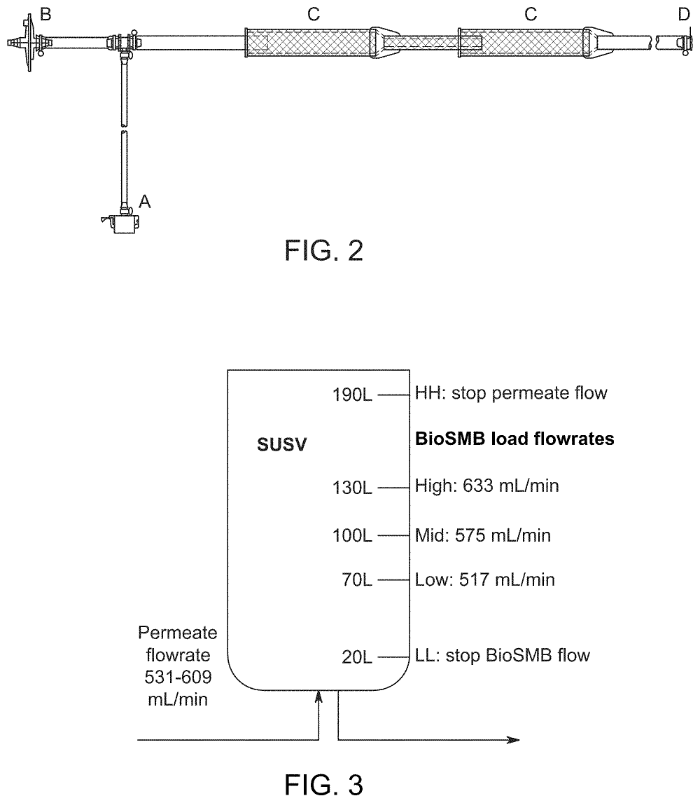

Aseptic operation of the inventive process was ensured by the use of either gamma-irradiated single-use components or pre-assembled autoclaved components throughout the entire connected flow path to provide bioburden control. Examples of gamma-irradiated components include: the Xcellerex® SUB bag, assemblies associated with the SUB and perfusion system, air break assembly (FIG. 2), sterilizing grade filter installed in the filter cart, the SUSV mixer bag, elution collection bags, the BioSMB manifold, and all the associated tote bags for media and buffer solutions. Examples of pre-assembled autoclaved components include hollow fiber filters and valve blocks connected to the chromatography columns to perform the resin sanitization procedures. The entire system boundary was maintained as a fully closed system through the use of disposable aseptic connectors, or by rendering the system functionally closed through the use of chemical cold sterilants.

Operation and Monitoring of the 500-L Single-Use Bioreactor (SUB). The control parameters, target setpoints, and allowable operating ranges of the 500-L SUB culture are listed below in Table 1B below.

TABLE 1B
General Production Operating and Performance Parameters.

| Control Parameter | Setpoint | | Operating Range | |
|---|---|---|---|---|
| Target Seed Density | 0.7 × 10⁶ | | ±0.2 × 10⁶ | |
| | cells/mL | | cells/mL | |
| Target Working | 450 | L | 400-500 | L |
| Volume | | | | |
| Initial Temperature | 36.8 | | ±0.5 | |
| Agitation | 152 | rpm | 142-162 | rpm |
| pH | 6.82 | | ±0.05 | |
| Dissolved Oxygen | 60% | | 20-90% | |
| Air Overlay | 5 | SLPM | ±1.0 | SLPM |
| Perfusion Start | 48 | hours | ±4 | hours |
| Perfusion End | 600 | hours | ±24 | hours |
| Temperature Shift | 144 | hours | ±24 | hours |
| Timing | | | | |
| Final Temperature | 36° | C. | ±0.5° | C. |

| | | |
|---|---|---|
| Cell Bleed Rate | On demand according to | N/A |
| | expected growth and | |
| | density target | |
| Glucose addition | On demand to 6 g/L | N/A |
| (50% w/v) | if bioreactor | |
| | glucose concentration | |
| | measurement ≤2 g/L. | |
| Sodium Carbonate | On demand to maintain | N/A |
| (1M) | pH at 6.82 | |

SLPM = standard liters per meter; rpm = revolutions per minute.

The production cultivation dissolved oxygen (DO) control and pCO₂ stripping strategy is represented in Table 2 below. Overlay was reduced to 0 SLPM when the Air to Tee Sparger increased to 10 SLPM. Additional air was added to tee sparge in 5 SLPM increments (no more than 10 SLPM total), when offline pCO2 was 152 mmHg. The 500-L single-use bioreactor (SUB) sparger Specifications were the following:

Tee sparger: 2-mm drilled hole; and agitator base: 2-μm sintered disc.

Table 2. Parameters for dissolved oxygen (DO) control strategy using one air mass flow controller (MFC) and two distinct oxygen (O₂) MFCs. SLPM=standard liters per meter.

TABLE 2
Parameters for dissolved oxygen (DO) control strategy using one air
mass flow controller (MFC) and two distinct oxygen (O₂) MFCs.
SLPM = standard liters per meter.

| 500-L DO | Air to Tee | O₂ to Tee | O₂ to Agitator |
|---|---|---|---|
| Output (%) | Sparger (SLPM) | Sparger (SLPM) | Base (SLPM) |
| 0 | 8.75 | 0 | 0 |
| 10 | 5 | 12.5 | 0 |
| 15 | 20 | 12.5 | 1.4 |
| 100 | 20 | 12.5 | 25 |

The 500-L (450-L working volume) culture was minimally sampled daily. Viable cell density, culture viability, offline pH, offline pCO₂, glucose concentration, lactate concentration, and osmolality were measured and recorded. Online agitation, temperature, pH, dissolved oxygen and backpressure were recorded, as were antifoam addition, air and oxygen gassing rates, cell bleed, and perfusion rates. Aseptic samples were also taken from the bioreactor and perfusion permeate for titer determination. The cell bleed was adjusted to maintain a viable cell density of about 50 million viable cells (MVC) per mL. This was done using a cell bleed calculator tool developed using Excel™ software (Microsoft). As the cell bleed rate was adjusted, the perfusion rate was also adjusted to maintain a total outflow rate not greater than 625 mL/min when perfusing at 2.0 working volumes/day.

Delivery of culture medium concentrates to the single-use bioreactor (SUB). The sterile perfusion culture medium was designed to be a concentrated stock solution that is room temperature stable. To meet these design requirements, the culture medium was separated in one embodiment into three sterile concentrated medium component solutions and an aqueous diluent component, each of which was stored in a single-use reservoir:

(concentrated medium component solution #1) 7.5×(w/w) concentrated medium solution (2× to 10× is typically useful, but the high end is determined by the composition of the medium and the quantity of dry ingredients that are to be added, so this depends on the media formulation);

(concentrated medium component solution #2) 20×(w/w) concentrated supplemental stock solution (CSSS; 20× to 100× concentrated supplemental stock solution is typically useful), containing cystine, tyrosine, and a surfactant;

(concentrated medium component solution #3) 50% (w/v) glucose; and

(4) water for injection (WFI) as aqueous diluent.

A schematic partial process flow diagram of the media concentrate delivery strategy at the 500-L scale is shown in FIG. 1A. The four components enumerated above were delivered directly to the bioreactor, relying on the agitation inside the bioreactor to mix the four components. The flow rates for the 7.5× medium, 20×CSSS, and 50% glucose concentrates were manually set using a calibrated peristaltic pump, and an in-line Sonotec IL. 52 flowmeter was used to monitor the flow rates and ensure accurate delivery. The aqueous diluent (water for injection (WFI)) was delivered on demand to the bioreactor to maintain the bioreactor level set point. The 7.5× media and 50% glucose solutions were delivered to separate ports at the top of the bioreactor. The WFI and 20×CSSS solutions were tied together to another port to minimize precipitation of the CSSS solution. In accordance with the invention, sub-surface addition of the different concentrated medium component solutions and aqueous diluent is preferably avoided. Delivery of all medium component solutions and aqueous diluent (e.g., WFI, 7.5× (w/w) concentrated medium solution, 20× (w/w) cystine/tyrosine/surfactant (CSSS) stock solution, and 50% (w/v) glucose) on demand, through separate ports, can also be accomplished using a ratio-controlled pumping skid and automation to maintain the culture volume in the perfusion bioreactor.

For the demonstration runs, perfusion was initiated on day 2 of production at 0.5 vessel volumes per day (vvd), ramped to 1 vvd at day 4, and 2 vvd at day 6. The inlet flow rates of the concentrated medium component solutions are shown in Table 3, below, along with estimated flow rates for WFI trim (average expected inlet flow rate). In Table 3, flow rates are also shown for the permeate flow rate prior to initiating cell bleed and at a couple of example cell bleed rates.

TABLE 3
Media inlet flow rates and cell bleed and permeate outlet flow rates at 500-L SUB scale.

| | Perf. | Cell | Inlet Flow Rates (mL/min) | | | | | Outlet Flow Rates (mL/min) | |
|---|---|---|---|---|---|---|---|---|---|
| Step | rate | bleed rate | Total | 7.5x | 20x | 50% | Est. | Est. | Est. |
| Change | (vvd) | (vvd) | inlet | media | CSSS | glucose | WFI | cell bleed | permeate |
| Day 2 | 0.5 | | 156 | 19 | 7.7 | 2.5 | 127 | | 156 |
| Day 4 | 1 | | 312 | 39 | 15 | 5 | 253 | | 312 |
| Day 6 | 2 | | 625 | 77 | 31 | 10 | 507 | | 625 |
| Example | 2 | 0.3 | 625 | 77 | 31 | 10 | 507 | 94 | 531 |
| cell bleed | | | | | | | | | |
| Example | 2 | 0.05 | 625 | 77 | 31 | 10 | 507 | 16 | 609 |
| cell bleed | | | | | | | | | |

Perfusion (Perf.) rates and cell bleed rates are expressed in vessel volumes per day (vvd);
WFI = water for injection

Operation of a first chromatography system and chromatography column sanitization. A first chromatography system was configured for capture of the recombinant therapeutic protein of interest into a protein isolate fraction in three exemplary demonstration runs. The first chromatography system included three Protein A affinity capture columns (14-cm diameter×5-cm height) in a Cadence™ BioSMB PD continuous single-use, multi-column chromatography system (herein also, “BioSMB”; Pall Life Sciences). MabSelect™ SuRe™ Protein A affinity matrix of highly cross-linked agarose resin (GE Healthcare Life Sciences) was used for the first two runs and Amsphere™ A3 Protein A chromatography resin (JSR Life Sciences) was used for the third run. The titer in the permeate was anticipated to be around 0.6 g/L, hence the loading was set at 83 column volumes (CVs) at 50 g/Lr loading for MabSelect™ SuRe™ and 108 CVs at 65 g/Lr loading for Amsphere™ A3. The method parameters are summarized in Table 4 below. The elution collection used a dynamic peak collection based on baseline to baseline absorbance at 280 nm wavelength (peak collection starting 0.1 absorbance units (AU) through peak and ending at 0.1 AU).

The BioSMB method was designed to allow the load flow rate to switch between a high, mid, and low flow rate. This toggling of flow rates helps manage the discrepant flows between connected unit operations, i.e. the permeate flow rate from the perfusion system, and the load flow rate of the BioSMB system. These demonstration runs were operated with fixed flow rate additions of the inlet media component solutions into the bioreactor, while the outlet flow rates for the cell bleed and permeate were modified on a daily basis. The range of potential cell bleed rates is shown in Table 3, thereby setting the range of permeate flow rates into the SUSV1 between 531-609 mL/min. The mid load flow rate for the BioSMB varied slightly between demonstration runs, but the high and low flow rates were set to ±10% of the mid flow rate and were set wider than the expected range of permeate flow rates. A schematic for the SUSV volume control is shown in Table 4, below, and FIG. 3, with exemplary flow rates used in one of the demonstration runs. A description of the automation used to toggle between the flow rates is in the next section.

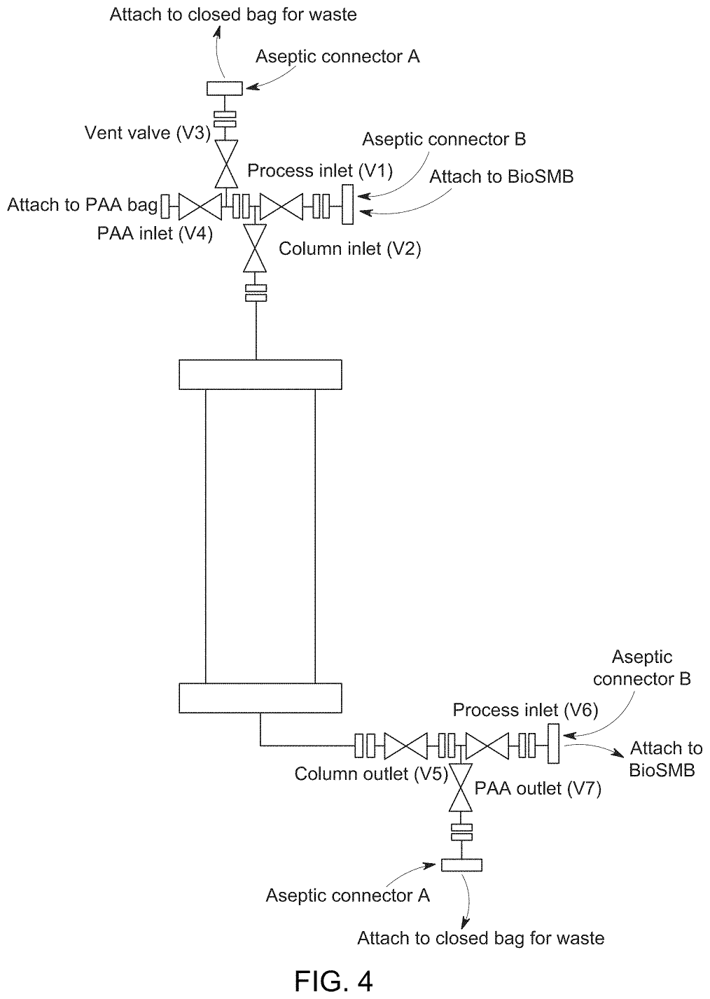

Prior to the start of the BioSMB capture step for each run, the resin was packed in glass column housings, and the resin and housings were chemically sanitized in an aseptic manner to render the BioSMB flowpath functionally closed. An autoclaved valve block assembly was attached to the inlet and outlet of each column housing, and aseptic connectors were used to attach the column to the BioSMB manifold, the sanitization solution bags, and the waste bags. A 30 mM peracetic acid (PAA) solution was used as the chemical sanitant, selected for its effectiveness as a sporicidal agent, but also mild enough to minimize any damage to resin function. (See, e.g., Jungbauer et al., Method for sterilizing liquid chromatography resins highly resistant to oxidation and a sterilization solution for use therein, U.S. Pat. No. 5,676,837). A schematic of the chemical sanitization setup for the column housing for one embodiment is shown in FIG. 4.

Briefly, column housings were packed with affinity chromatography resin (open to air). Valve blocks were autoclaved. In reference to FIG. 4, each column off-line of the BioSMB skid was treated in the following manner: PAA was primed into a single-use bag attached to vent valve (in FIG. 4, V4 to V3) using a stand-alone peristaltic pump. PAA was flushed through PAA Inlet and Outlet valves into a single-use collection bag attached to aseptic connector A for 3 column volumes (CVs) (in FIG. 4, V4 to V2 to V5 to V7). PAA was held in each column for ≥15 minutes, then the columns were flushed again with 3 CVs of equilibration buffer (EQ) or storage buffer (in FIG. 4, V4 to V2 to V5 to V7). Then each column was attached to the chromatography skid through the Process Inlet (in FIG. 4, V1) and Process Outlet (in FIG. 4, V6) via aseptic connector B connectors. After this the skid lines were primed through the Vent Valve into a a single-use bag (in FIG. 4, V1 to V3), and the columns were ready for a BioSMB run, and rendered functionally closed closed with this sanitization procedure.

Alternatively, PAA sanitization of packed chromatography columns can be performed off-line as described above, but with a flush of EQ or storage buffer performed on the simulated moving bed (SMB) skid (in FIG. 4, flush from V1 to V2 to V5 to V6). A single-use bag containing the PAA solution can be attached to the column inlets (in FIG. 4, V2), and the sanitization and flush procedure can all be done with the columns on the SMB skid. Sanitants other than PAA can be used instead, but these must be sporicidal. (See, e.g., Jungbauer et al., Method for sanitizing liquid chromatography resins highly resistant to oxidation and a sterilization solution for use therein, U.S. Pat. No. 5,676,837; Monie et al., Sanitization method for affinity chromatography matrices, WO2016/139128A1 and US2018/036445A1).

The chemical sanitization procedure during the demonstration runs was only performed once at the beginning of each run. The Protein A affinity chromatography method itself used a 0.1M NaOH regeneration cleaning procedure, but this sanitant was not expected to be strong enough to have bacteriocidal and sporicidal capabilities. For the demonstration runs, the chemical sanitization procedure was performed offline of the BioSMB skid, however, the sanitization procedure can be performed on the skid with the BioSMB manifold.

TABLE 4
Exemplary first chromatography system parameters for Protein
A capture of a recombinant therapeutic protein of interest
(shown for Run 2). In this example, the load flow rate
was 585 mL/min for an 83 CV load, with a 2.6 minute total
residence time, and a cycle time of 5.5 hours.

| | | | Approx. | |
|---|---|---|---|---|
| | | Volume | Flow rate | Switch |
| Step | Solution | (CV) | (mL/min) | Time |
| Loopback | Flowthrough from | 83 | 585 | 1.0 |
| | 1^{st} column load | | | |
| Feed | Harvest fluid | 83 | 585 | 1.0 |
| Wash 1 | EQ | 2 | 156 | 0.09 |
| Wash 2 | High salt pH 7.5 | 2 | 156 | 0.09 |
| Wash 3 | EQ | 3 | 156 | 0.14 |
| Elution | Acetate pH 3.6 | 4 | 156 | 0.18 |
| Strip | Acetic acid | 3 | 156 | 0.14 |
| Flush | EQ | 1 | 156 | 0.05 |
| Regeneration | Sodium hydroxide | 3 | 156 | 0.14 |
| Equilibration (EQ) | EQ | 4 | 156 | 0.17 |

Automation and Single-Use Surge Vessel Volume Control. A process automation system (PAS) was employed that provides flexible process control and management of the skid-based and portable production equipment in support of scalable continuous capture biologic production campaigns. The automation also provides for autonomous batch reporting, data collection, and materials tracking. It was a reusable class-based design and architecture that can be rapidly deployed across production facilities of the same class and configuration. A high-level process automation overview, depicting communication between equipment types, is shown in a schematic representation of an embodiment of the invention (FIG. 5).

FIG. 5 shows a schematic representation of various hardware and software components of an exemplary embodiment of the inventive automated facility for manufacturing a purified therapeutic protein drug substance that enable communication of data between the different components of the system. In particular, FIG. 5 illustrates a number of connection interfaces (e.g., Profibus drops) included in the skids of the single-use bioreactor system, the perfusion system, and a continuous first chromatography system. The connection interfaces can provide logical connections and/or physical connections between components of the system. In situations where the interfaces enable physical connections, the connection interfaces can be connected to hardware components, such as ethernet/Internet Protocol (IP) gateways. One or more dongles can be coupled to the portable pieces of equipment, such as the filter bank, the first mix tank, and the second mix tank. The dongles can store and/or communicate information related to the control of the portable pieces of equipment to the production facility control system. In certain situations, one or more of the portable pieces of equipment can internally store the information stored on the dongles and can function as an internal dongle.

In the illustrative example of FIG. 5, the various devices can communicate using one or more Profibus communication protocols. In various implementations, the control of the perfusion system and the continuous first chromatography system can be configured to be set and/or adjusted based on information related to the operation of at least one optional unit from among a Filter Bank, a Feed Tank A, a Feed Tank B, a Collection Tank A, and a Collection Tank B. In the illustrative example of FIG. 5, Collection Tank A can have a logically derived software connection with Dongle 1 coupled to a first portable mix tank. Additionally, the skid of the continuous chromatography system can have physical connections via gateway devices to the filter bank and the first portable mix tank. Dongle 2 can be coupled to the filter bank and provide information regarding the operation and identifiers of the filter bank. In certain situations, data related to the operation of the filter bank can be used in the control of the continuous chromatography system. Further, Dongle 3 can be coupled to the second mix tank and provide information related to the operation and identifiers of the second mix tank. For example, Dongle 1 can indicate that the first mix tank functions as a collection tank for the perfusion system, while Dongle 3 can indicate that the second mix tank functions as a collection tank for the continuous chromatography system.

While the illustrative example of FIG. 5 indicates various software connections and physical connections between components of the inventive automated facility for manufacturing a purified therapeutic protein drug substance, it should be understood that the physical connections can be replaced by software connections in particular additional implementations of the purified therapeutic protein drug substance production line, while some of the software connections can be implemented as hardware connections in some additional implementations of the purified therapeutic protein drug substance production line.

Briefly, the automation for the SUSV1 level control relies on the specification of pre-set volume range limits upon which a control action is taken. For example, in FIG. 3, when the volume in SUSV1, or any other SUSV in the continuous or semi-continuous process flow, e.g., SUSV2 or SUSV3 or SUSV4 or SUSV5, etc. (“SUSV” in FIG. 3), reaches the low and high volume alarms, the SMB (or other process skid, e.g., viral filtration or UF/DF skid) automatically switches to its low and high flow methods, respectively. Since the low and high flow rates for the SMB are chosen to be outside of the range of expected permeate flow rates going into the SUSV1, the result is that the SUSV1 volume is driven back to the center point volume. Once the center point volume is attained, the SMB flow rate reverts to its mid flow rate method. When the SUSV1 reaches the low low (“LL”) alarm, the SMB flow rate is stopped; conversely, when the SUSV reaches the high high (“HH”) alarm, the perfusion permeate flow rate is stopped.

Operation of the 2-L Bioreactor Satellites for Comparison to 500-L Bioreactor. Bioreactor satellites were conducted in 2-L autoclavable glass bioreactors (Applikon) using a BPS-i100 perfusion system (Levitronix). A sterile bag was used to transfer cell culture from the 500-L SUB to the 2-L bioreactor targeting the inoculation cell density. All concentrated medium component solutions and WFI were sourced from the manufacturing facility and reconstituted to the 1× formulation. Two different 1× formulations were made at 8 g/L and 12 g/L glucose to accommodate the range of cell density during production. Satellites were controlled to the same target setpoints as the SUB or scaled down accordingly. O₂, CO₂, and air flow were controlled using rotameters, and overlay and air sparge flow rates for CO₂ stripping were scaled by vessel volumes per minute (VVM). Agitation was scaled by power per unit volume (P/V). The pH was controlled using a ±0.02 deadband. Two 0.02 m² hollow fiber perfusion filters (0.2 μm pore size) were used in parallel and Run 1 matched the permeate flux through the filter by recycling permeate back into the bioreactor. The permeate recycle was abandoned for subsequent runs for ease of operation. Cell density at both large and small scale was controlled using a cell bleed calculator. This equation used the current and previous day's offline cell counts to calculate the apparent growth rate and the required cell bleed rate needed to control at a specified target viable cell density.

Results and Discussion

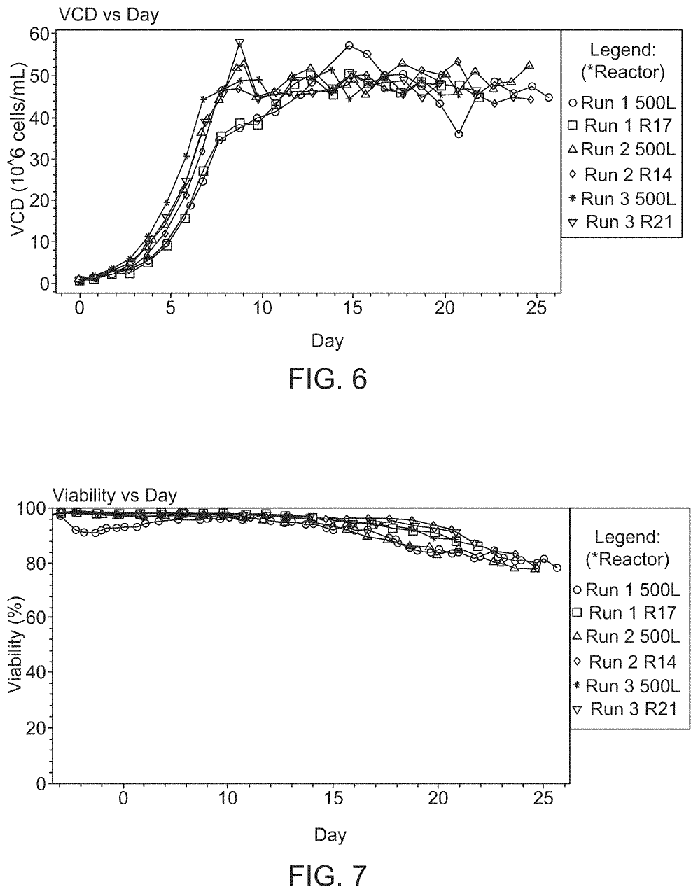

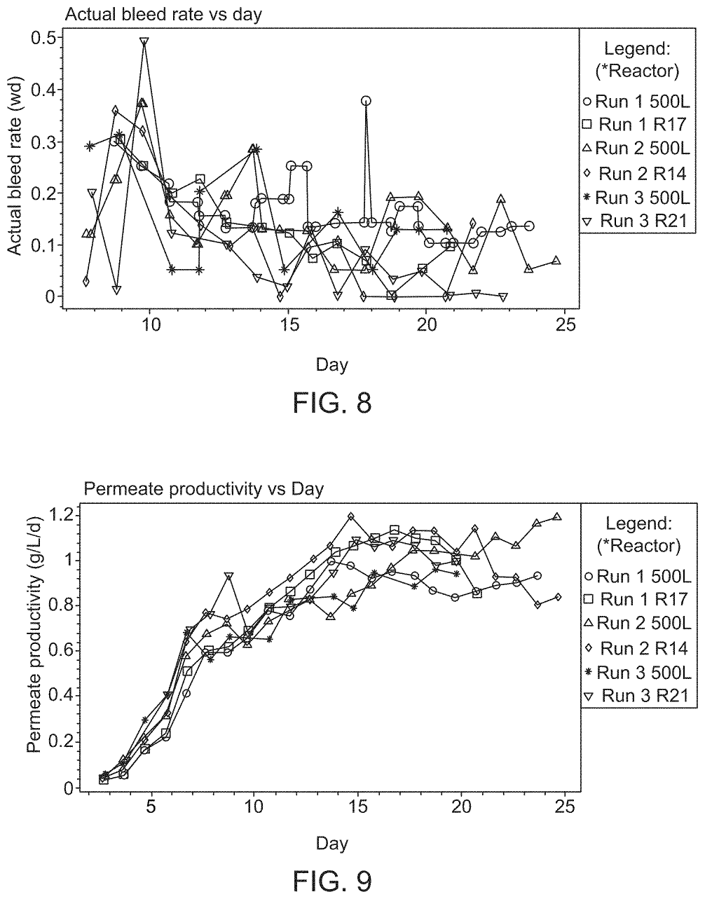

Performance of Continuous Perfusion Culture. Cell culture performance results are presented for the three 500-L demonstration runs and a corresponding 2-L satellite run: viable cell density (VCD) is shown in FIG. 6; viability is shown in FIG. 7; cell bleed rate is shown in FIG. 8; and permeate productivity is shown in FIG. 9.

The cell density was successfully controlled to a target of approximately 50 million viable cells/mL (MVC/mL), with higher bleed rates used at the beginning of the culture and tapering down to a lower bleed rate over the culture duration. A slightly different cell bleed strategy was used in Run 1, which resulted in a more variable growth profile. Later runs moved to a cell bleed strategy based on the previous day's growth rate, which resulted in a more tightly controlled VCD. Viability was maintained above 70% for the duration of the cell culture up to 26 days. The permeate productivity achieved around 1 g/L/day for this cell line and process, and the perfusion filter was able to maintain high product passage for the entire duration of the run (data not shown).

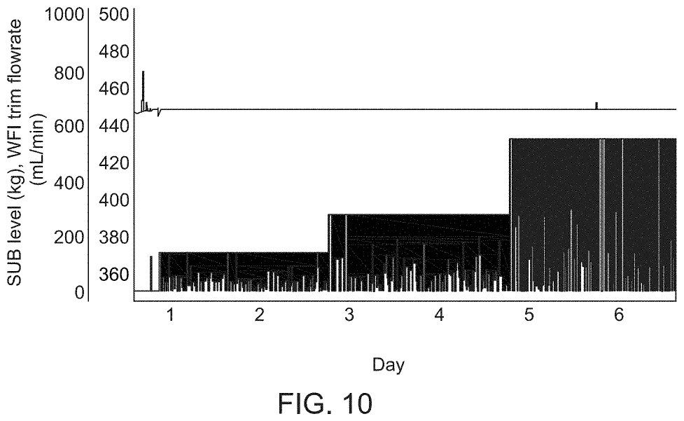

Performance of Media Concentrate Delivery. Multiple performance markers were assessed to evaluate the accuracy of the media concentrates delivery at 500 L scale. First, flow rate verifications were performed for the individual media component solutions to ensure that the in-line flowmeter was providing accurate readings. Second, FIG. 10 shows that the SUB level control operated as intended, with the culture volume in the bioreactor maintained at 450 L, and the WFI trim flow rate turning on as needed to maintain the culture volume. In addition to these operational checks, the cell culture data shown in FIG. 6, FIG. 7, FIG. 8 and FIG. 9 indicate similar performance with respect to cell growth, viability, and productivity between the 500-L scale operated with concentrated culture medium component solutions and the 2-L comparator satellite operated with 1× delivered culture medium.

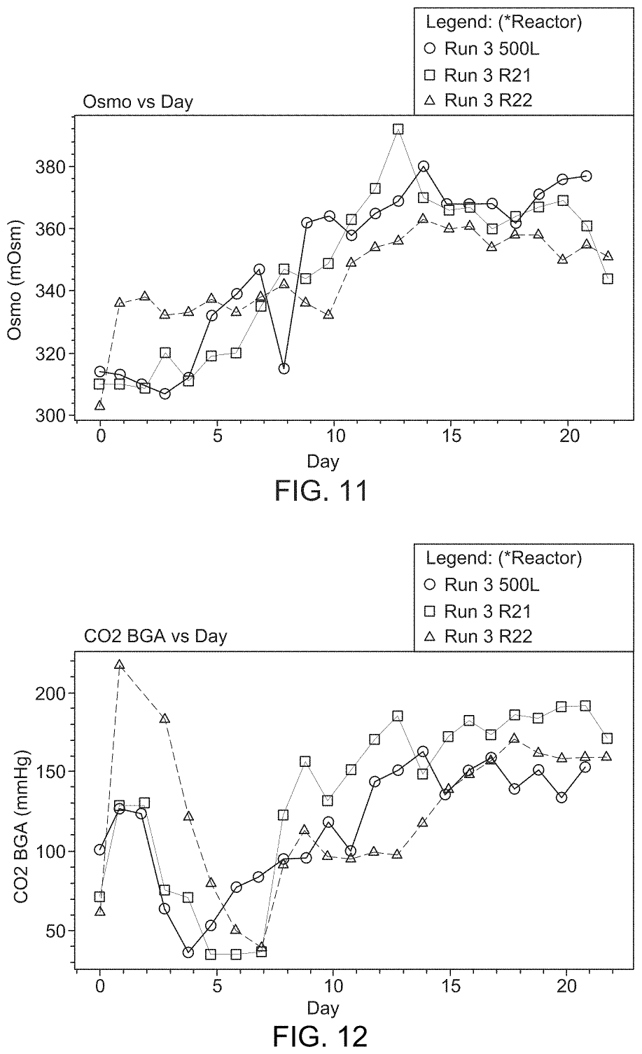

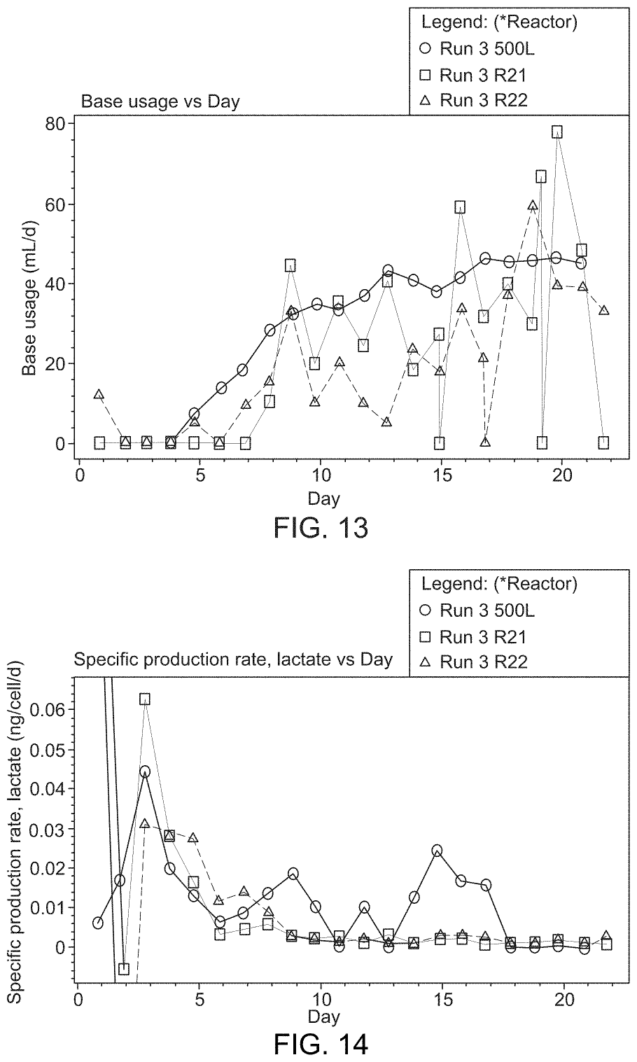

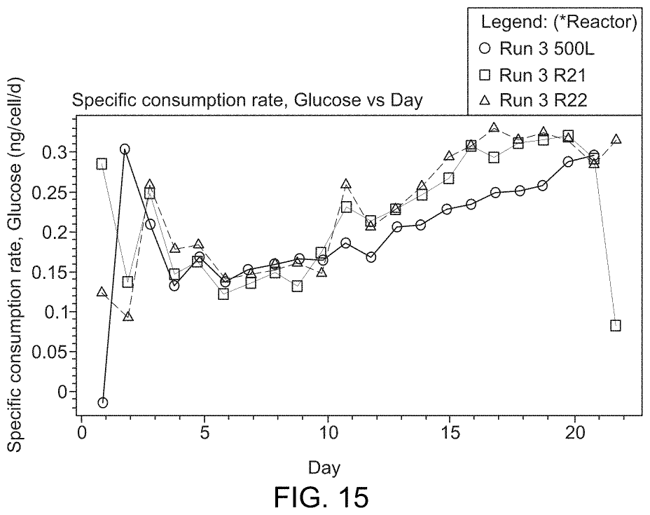

Additional metabolic data are presented, comparing the 500-L Demonstration Run 3 to its 2-L comparator satellites: osmolality in FIG. 11, CO₂ levels in FIG. 12, base usage for pH control in FIG. 13, specific lactate production in FIG. 14, and specific glucose consumption in FIG. 15. All of these trends show similar performance between the 500-L scale operated with concentrated culture medium component solutions and the 2-L satellites operated with 1× delivered culture medium, with particular emphasis on the osmolality profile (see, FIG. 11), which is an indicator of the addition of medium component concentrates to the bioreactor and consumption by the cells.

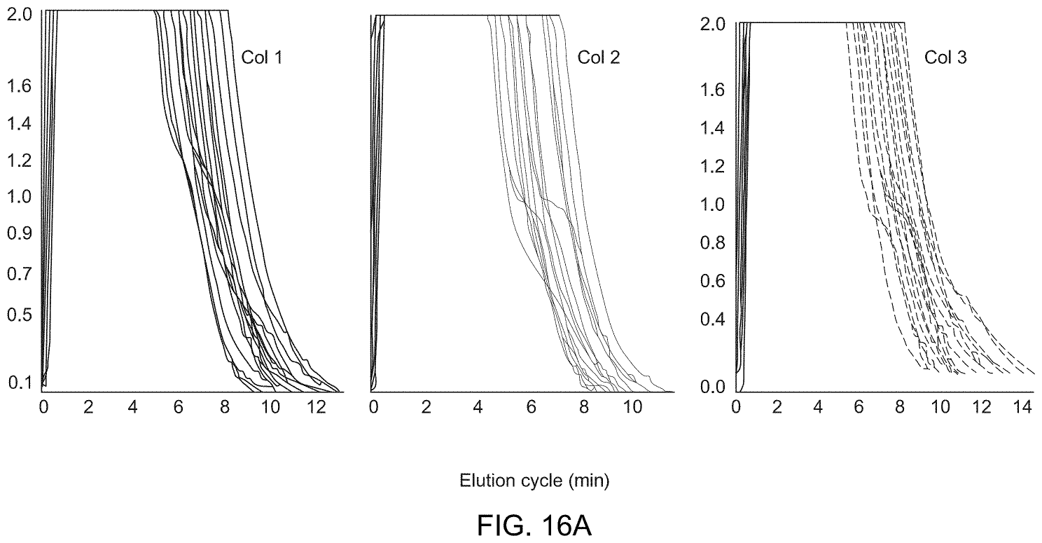

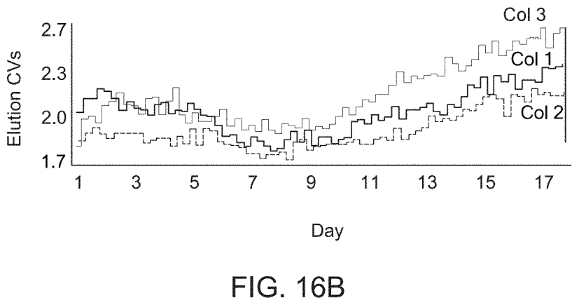

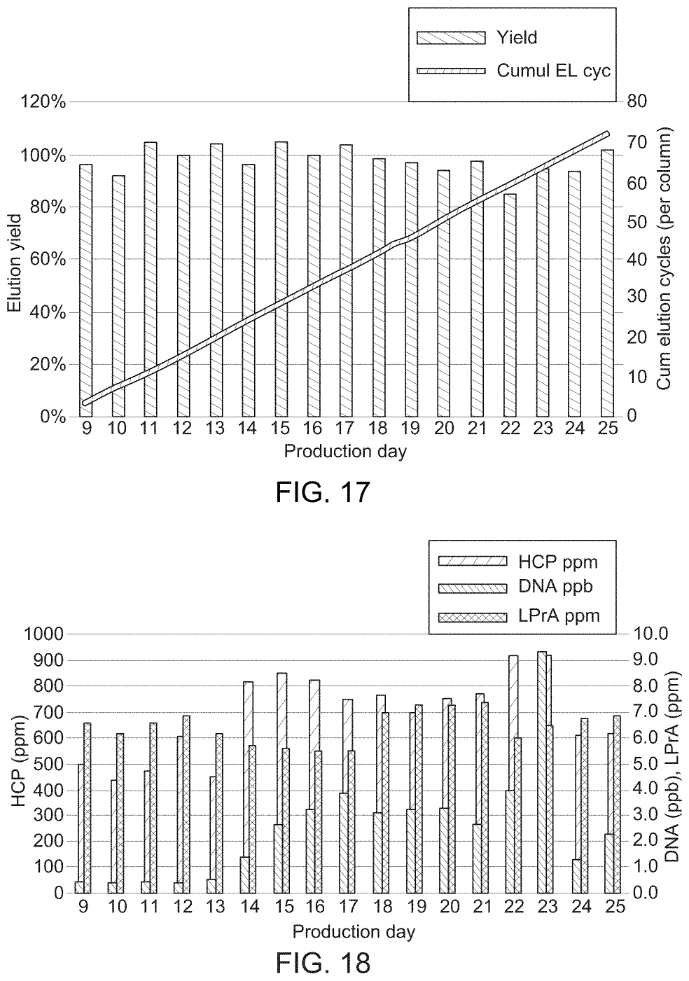

Performance of Continuous Capture Simulated Moving Bed (SMB) first chromatography system. Simulated Moving Bed (SMB) first chromatography system BioSMB continuous capture performance results are presented for Demonstration Run 2, which had the longest operating duration of the three runs, completing a total of 72 cycles per column (216 total completed elution cycles) over 17 days of continuous operation. The Protein A elution ultraviolet (UV) absorbance (A280) profiles are shown as a daily snapshot of each column (FIG. 16A), along with the elution column volumes (CVs) for every elution cycle (FIG. 16B). The elution peak width was similar between the three columns, but all columns showed some peak broadening towards the later part of the run. This could be due to increased permeate titer, and therefore elution mass, over the course of the run duration, but also could be attributed to changes in column performance with resin age. The resin in this run had previously seen 60 cycles, so the total cycles at the end of Run 2 was at 132. The Protein A step yields, shown as the combined daily pool of elution cycles in FIG. 17, were similar over the course of the 72 cycles. Process related impurities of the combined daily elution pools (neutralized to pH 5, and 0.2-μm filtered) are shown in FIG. 18. The level of impurities was relatively consistent between daily elution pools, indicating consistent performance of the resin over its lifetime.

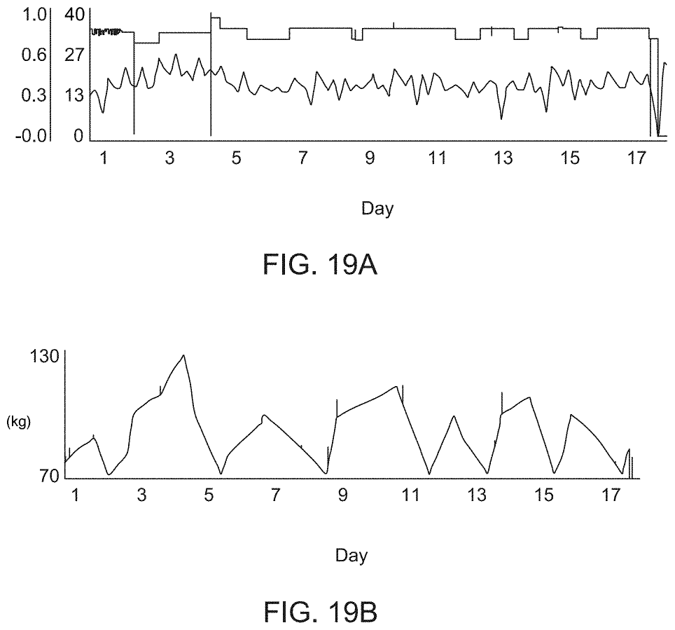

The level control of the SUSV and corresponding changes in load flow rate on the BioSMB are shown in FIG. 19A-B. Following the trends of the SUSV1 volume and the BioSMB load flow rate, when the SUSV1 reached the low volume of 70 L, the BioSMB flow rate shifted to the low flow rate set point, and at the high volume of 130 L, the BioSMB flow rate shifted to the high flow rate set point. After the lower or higher BioSMB flow rate drove the SUSV1 volume back to 100 L, the flow reverted to the center point.

Bioburden Control. Bioburden and endotoxin testing were performed at multiple sample locations over the duration of the runs. Results from Demonstration Run 2 are shown in Table 5. For this run, the production bioreactor was operated for 25 days, and the first chromatography system (here, BioSMB) was operated for a total of 17 days. Perfusion was started on day 2, the Protein A columns were sanitized on day 7, the BioSMB was started on day 8, and the first neutralized Protein A pool was sampled on day 9. The bioreactor sample was whole broth from the bioreactor, the PERM sample was taken from the permeate side of the perfusion filter, the SUSV1 was sampled directly from the vessel, the NPRA was sampled directly from the daily neutralized elution collection vessel, and the 2ndP was sampled from the BioSMB outlet for the second pass flow-through. All bioburden results were negative (0 CFU/10 mL). All endotoxin results were negative, except for the day 18 second pass flow-through which showed some gel clotting at the lowest dilution. The system had clean endotoxin results in the days preceding and following that sample, and the corresponding bioburden sample was clean, so this could have been a sampling issue.

These results confirm that a closed aseptic system boundary was maintained from the single-use bioreactor—and its associated plurality of different concentrated medium component solutions and an aqueous diluent, to the BioSMB and its associated columns, buffer solutions, and elution vessels. Furthermore, the results validate using a single-use air break drain for the permeate and chromatography system waste lines, and the sporicidal sanitization of the columns and resin using a peracetic acid sanitant.

To summarize, the demonstration runs described above showed consistent performance of a 500-L continuous perfusion process, demonstrating ability to maintain target viable cell density (VCD) out to at least 26 days with high viability and high product passage through the perfusion filter. Accurate delivery of media concentrates was demonstrated, i.e., there was comparable performance between 500-L SUB operating with a plurality of different concentrated medium component solutions and an aqueous diluent and 2-L comparator satellite bioreactors operating with 1× culture medium delivery. There was consistent performance of the first chromatography system, including Protein A affinity chromatography capture step, with respect to elution yields and impurity levels. The operation of the automated SUSV1 volume control via flow rate toggling of the first chromatography system load was demonstrated, as was the single-use air break drain.

Employing the inventive process and automated facility enables one to maintain an aseptic closed system. The practice of the invention is facilitated by the use of single-use components, e.g., single-use bioreactors, single-use mixer bags, single-use assemblies, single-use simulated moving bed flow path—and the demonstrated ability to render the system functionally closed and aseptic via the use of a sporicidal sanitization method for the chromatography resin and column housings of the chromatography systems.

TABLE 5
Bioburden and endotoxin results from Run 2. The production
cultivation period of the SUB was 25 days, with continuous
capture chromatography by BioSMB for 17 days starting on day
8; bioburden. Perfusion started on day 2, Protein A columns
were sanitized on day 7; first NPrA pool was collected on day 9.
Endotoxin (EU/mL)/Bioburden (CFU/10 mL)

| Day | BRX | PERM | SUSV | NPRA | 2ndP |
|---|---|---|---|---|---|
| 8/9 | <3/0 | <0.6/0 | <0.6/0 | <0.6/0 | <0.6/0 |
| 14 | <3/0 | <0.6/0 | <0.6/0 | <0.6/0 | <0.6*/0 |
| 18 | <0.6/0 | <0.6/0 | <0.6/0 | <0.6/0 | <4.8/0 |
| 21 | <3/0 | <0.6/0 | <0.6/0 | <0.6/0 | <0.6/0 |
| 25 | <3/0 | <0.6/0 | <0.6/0 | <0.6/0 | <0.6/0 |

BRX = bioreactor; PERM = permeate; SUSV = single-use surge vessel (SUSV1); NPRA = neutralized Protein A; 2ndP = second pass flow-through.
*= 2^{nd} pass flow-through was tested separately for columns 1, 2, and 3; all values were <0.6 EU/mL.

### Example 2. Viral Inactivation and Neutralization Systems and Further Downstream Processing

After Protein A affinity chromatography via the first chromatography system, the downstream unit operations can be run in a batch mode or continuously or semi-continuously.

In one exemplary embodiment. a two-tank automated viral inactivation system was connected to the elution outlet of the BioSMB system to evaluate a batch low pH viral inactivation step operated with a continuous inlet flow. The elution collection from the BioSMB Protein A affinity chromatography (first chromatography system) alternated between two 50-L single-use mixer vessels. When the volume in the one vessel reached its predetermined value, in this case a target of around 20 L, the acid titration for the low pH viral inactivation process was initiated in that vessel, and elution collection was switched to the other vessel. For the batch viral inactivation operation, 2 M acetic acid was added to a target pH 3.5 in a stepwise fashion, with both mixing of the vessel with a bottom agitator and a recirculating pump. The pool was incubated for 60 minutes, then 2 M tris(hydroxymethyl)aminomethane (“Tris”) base was added to a target of pH 5.0 in a similar manner to the acid titration. The neutralized virally inactivated product pool was subsequently transferred out of the system. A total of six cycles were performed, three in each tank, and both the low pH and neutralization setpoints were achieved within the target pH range of ±0.1. The titration duration for acid and base addition was each about 10 minutes.

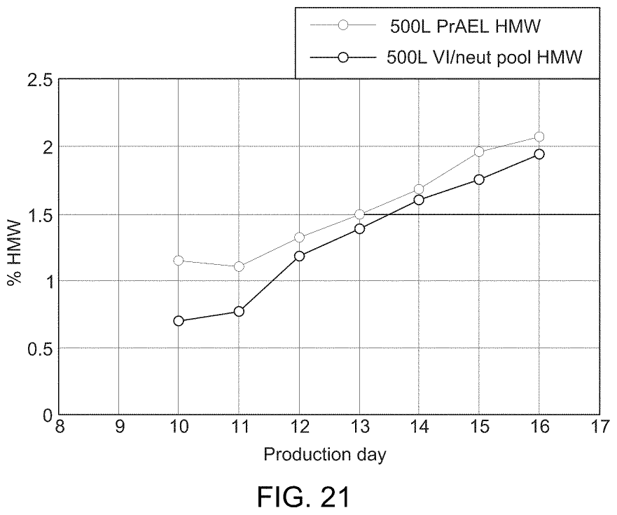

FIG. 21 shows comparison of high molecular weight (HMW), as measured by SE-HPLC, between the post-Protein A chromatography protein isolate fraction and the low pH viral inactivated and neutralized (VI/Neut) virally inactivated product pool. The viral inactivation step was performed manually on production day 10, whereas days 11-16 were performed on the automated system. The results in FIG. 21 show that the high molecular weight (HMW) between the post-Protein A chromatography protein isolate fraction and the post-VI/Neut virally inactivated product pool is comparable, indicating that the acid and base titration operation of the viral inactivation system did not impact the product quality.

The following is another non-limiting example of downstream batch mode operations, wherein all steps use single-use (disposable) components:

Several days of BioSMB (first chromatography system) operations, involving Protein A affinity chromatography, are pooled in a protein isolate fraction for viral inactivation (VI). Viral inactivation is performed by lowering the pool to pH 3.5 with 2 M acetic acid. The acidified pool is held for 1 hour. After the low pH hold, the virally inactivated product pool is neutralized to pH 5.0 with 2 M Tris base. The neutralized virally inactivated product pool is filtered through Millistak+® HC Pod A1HC depth filters (MilliporeSigma), and a subsequent sterilizing grade filter to clarify the virally inactivated product pool prior to introducing it into the second chromatography system.

The second chromatography system comprises a cation exchange (CEX) column in flow-through mode. This step utilizes Fractogel® EMD (M)COO— resin (MilliporeSigma). CEX is followed by further chromatographic purification via a third chromatography system, such as, but not limited to Mixed Mode Chromatography (MM) with Capto™ Adhere (GE Healthcare Life Sciences) resin. A pH and/or conductivity load conditioning can be performed prior to loading on the MM column (in-line or batch). This step is performed in a flow-through mode.

Viral Filtration (VF) is performed on the MM flow-through pool as an orthogonal method for removing virus particles by size to obtain a virus-free filtrate comprising the recombinant therapeutic protein. Viral filtration can employ, for example, but not limited to, a Planova™ 20N filter (Asahi Kasei Corporation). The virus-free filtrate pool is concentrated and diafiltered using, for example, but not limited to, 30-kD regenerated cellulose filters (MilliporeSigma) to place the purified therapeutic protein drug substance in the formulation buffer at the target product concentration.

### Example 3. Peracetic Acid (PAA) Sanitization of Protein A Matrix

A successful continuous process can be facilitated by extending the sterile envelope from the perfusion bioreactor to the Protein A affinity chromatography capture step (or first chromatography system) and into the downstream process through use of sterile, single-use components and equipment. However, it is difficult to assure the sterility of chromatography columns that are packed in-house, and gamma irradiated columns are expensive and not widely available commercially. To address the need for sterile chromatography columns, we developed a chemical column sanitization process that allows the capture step to run continuously in a sterile manner, assuming the use of aseptic connectors and with thorough inspection of the integrity of any welds in the system(s).

Materials and Methods. The materials listed in Table 6 were employed in the packing and sanitization procedures described in this Example 3; rehydrated inoculants of individual bacterial species listed in Table 6 were prepared according to manufacturer's instructions from BioBall® Multishot 10E8 kits (bioMérieux SA), having a mean of between 0.7 and 1.5×10⁸ cfu with a standard deviation of ≤20% of the mean. Re-hydration was into 1.1 mL of BioBall® Re-Hydration Fluid to provide 10×100 μL doses of 10⁷ cfu.

TABLE 6
Materials.

| | |
|---|---|
| Protein A: | MabSelect ™ SuRe ™ Protein A affinity matrix |
| | (GE Healthcare Life Sciences) |
| Columns/equipment: | BPG 140/500 Column Housing |
| | (GE Healthcare Life Sciences) |
| | 2-L Applikon Bioreactor |
| Buffers/Media: | 25 mM Tris, 100 mM NaCL, pH 7.4 |
| | Peracetic Acid, 35% v/v (Pfaltz & Bauer) |
| | Water (Milli-Q ®-purified) |
| | 0.1M NaCl |
| | 1M NaOH |
| | 2% Benzyl Alcohol, 50 mM Citrate, pH 5.0 (an |
| | exemplary storage buffer) |
| | BAK004-067 medium |
| Bacterial inoculants: | Bacillus subtilis spores |
| | Aspergillus brasiliensis |
| | Candida albicans |
| | Escherichia coli |
| | Pseudomonas aeruginosa |
| | Staphylococcus aureus |
| | BioBall ® Rehydration Fluid (1.1 mL) |

Primary column packing and sanitization procedure. Ethanol (70% v/v) was added to the empty column housing. Air was removed from under the bottom frit by sucking it through the column outlet with a peristaltic pump and by manually directing air bubbles with a small paddle. Once all air was removed, the appropriate amount of MabSelect® SuRe Protein A resin to pack either a 5-cm or 10-cm resin bed was added to the BPG 140/500 glass chromatography column housing. A peristaltic pump was used to remove the storage buffer through the outlet of the column. The storage buffer shown in Table 6 was only an example; alternatively, the storage buffer can be whatever buffer the resin was shipped in, e.g., a buffer containing 20% ethanol, or it can be EQ or other buffer or deionized water, if settling/decanting of the resin was previously employed to remove fine particles or get an accurate slurry percentage.

Approximately 3 column volumes (CV, based on the settled bed height) of either Milli-Q®-purified water or Protein A equilibration buffer (EQ) were added to the top of the settled bed. This volume was drawn through the settled bed with a peristaltic pump to remove residual storage buffer. Approximately 2CV of Milli-Q®-purified water or EQ buffer was added to the settled bed and the bed was slurried slowly with a small paddle. To ensure a level of bioburden and demonstrate the effectiveness of the sanitization Procedure, B. subtillis or a cocktail of organisms (see, Table 6) was added to the slurry at approximately 100 CFU/mL of slurry volume. A peristaltic pump was used to settle the bed and remove excess volume.

Approximately 3 CV of 0.7% (v/v) PAA was added on top of the settled bed; this volume was drawn through the settled bed with a peristaltic pump to remove residual water (or Protein A EQ). Peracetic acid (0.7% v/v) was added to the column to produce an approximately 50% slurry of resin and PAA. The resin was slurried with a paddle. The resin slurry was allowed to settle long enough to produce a layer of liquid on top of the bed large enough to cover the column top adapter to above the adapter O-ring. The top column adapter was put in place and lowered until the O-ring was covered by the PAA solution. The top adapter was manipulated to remove residual air from the frit and O-ring. The top adapter was allowed to soak in the 0.7% PAA solution for approximately 20 minutes and then the o-ring was tightened. The top adapter was lowered into the PAA solution to force PAA solution up through the central column tube. The central column tube was then connected to the 0.7% PAA packing solution. Columns were packed at between 380 and 550 cm/hour with 0.7% PAA solution, although higher rates of packing are also possible, e.g., 600 cm/hour.

The following procedure was used to mimic steps a packed column would undergo in a good manufacturing practices (GMP) production run. All steps were performed at a flow rate of approximately 150 cm/hour. Ten CV of deionized water was flushed through the column to remove the PAA solution. Three CV of sterile 0.1M NaCl was flushed through the column to simulate Height Equivalent to the Theoretical Plate (HETP) testing. Three CV of storage buffer was flushed through the column and the column was stored overnight before sanitization with PAA.

The column was sanitized with 0.2% (v/v) PAA. Five CV of 0.2% PAA was flushed through the column in the down flow direction, and 5 CV of 0.2% (v/v) PAA was flushed through the column in the up-flow direction. The column was held in 0.2% PAA for 60 minutes, after which Protein A EQ buffer was flushed through the column to remove the PAA solution. A sample was taken at 5 CV and 7 CV and submitted for bioburden analysis. The column was then connected to a 2-L bioreactor containing BAK media. The media was recirculated through the column at approximately 50 mL/min with a peristaltic pump. Inlet pressure of the column was monitored with a SciLog® (Parker Hannifin Corp.) pressure transducer. The pump was set to turn off, if a maximum pressure differential of 20 pounds per square inch differential (psid) was reached. Columns were left connected to the reactor for 10 to 14 days. A post-recirculation sample was pulled off the column upon completion of the experiment and submitted for bioburden analysis.

Alternative Packing and Sanitization Procedure 1. An alternative packing and sanitization procedure was also used, following the primary packing and sanitization procedure described above, except that 0.7% (v/v) PAA was used for slurrying, packing, and sanitization.

Alternative Packing and Sanitization Procedure 2. A second alternative packing and sanitization procedure was also tried, which used 0.2% (v/v) PAA with 0.1 M NaCl for slurrying and packing the column. The column was sanitized with 0.2% PAA after packing, as in the primary packing and sanitization procedure described above, and the sodium chloride was added in an effort to improve the packing performance. However, the sodium chloride interacted with the PAA solution causing air on the column. The addition of sodium chloride together with PAA is therefore not recommended.

Alternative Packing and Sanitization Procedure 3. In a third alternative packing and sanitization procedure, the resin was slurried and packed in 0.1 M NaCl without PAA. The resin was spiked with the bioburden cocktail (see Table 6) to approximately 120 CFU/mL during the initial slurry step, prior to packing. The 0.1 M NaCl was removed by flushing approximately 3 CV of water (or ProA EQ) through the column, then the column was sanitized with 0.2% (v/v) PAA (5 CV down, 5 CV up, 60-minute hold) and flushed with Protein A EQ buffer prior to being connected to the 2-L bioreactor. Flush samples and post-bioreactor recirculation samples were pulled off the column upon completion of the experiment and submitted for bioburden analysis, as in the previous experiments that employed the primary sanitization procedure (above). For bioburden testing about 100 to 200 mL of sample were pulled into sterile sample bags, and bioburden was analyzed as described below.

Process scale confirmation of packing and sanitization procedure. Two 500-L engineering runs that utilized the BioSMB to perform continuous capture of an immunoglobulin of interest were performed in a manufacturing plant. This production process used four, 14-cm diameter by 10-cm bed height MabSelect™ SuRe™ Protein A affinity matrix columns for the capture of the bioreactor permeate. The first run used the primary packing and sanitization procedure of slurrying the resin in 0.7% (v/v) PAA and packing the column with 0.7% (v/v) PAA followed by a 0.2% (v/v) PAA sanitization step. The columns were not repacked for the second run. The second run used only the 0.2% PAA sanitization step prior to putting the columns in service.

Bioburden analysis. Bioburden testing was performed as per USP<61>. Briefly, a ten (10) mL of sample was aseptically withdrawn from a sample bag containing 100 mL of total drawn sample and was added to at least 90 mL of sterile phosphate buffered saline (PBS) or sterile water (or such volume of sample and diluent so the product was not diluted greater than 1:10). The total aliquot volume was funneled into a Milliflex® filtration system (MilliporeSigma), filtered, and then incubated on Milliflex® agar plates at 30-35° C. for tryptic soy agar (TSA) and 20-25° C. for Sabouraud Dextrose agar (SabDex; SDA) for greater than or equal to 3 days and greater than or equal to 5 days, respectively.

Results. Table 7 (below) contains the results for each of the test conditions employed in column sanitization experiments. All conditions tested resulted in a fully sanitized column. No bioburden was seen in any flush sample, and all conditions resulted in at least 10 days of sterile operation when connected to the 2-L bioreactor. Ten days was the minimum time that a column was connected to the 2-L bioreactor. The 10-day incubation period was selected because it would allow detectable growth on the chromatography column and in the bioreactor for even slow growing microorganisms.

The 0.7% (v/v) PAA slurry and packing procedure (i.e., the primary column packing and sanitization procedure described above) was seen as the most stringent option for sanitization at process scale and was chosen for use in the engineering runs. A post-column performance test of the 0.2% (v/v) PAA sanitization step was done after connecting the columns to the BioSMB valve block, because 0.2% PAA has been shown to be effective against spore forming bacteria and all resin and column parts had been in contact with 0.7% PAA during the packing process. A full assessment of the impact to Protein A lifetime with 0.7% PAA exposure had not been performed at the time of these experiments, so the lower concentration was also chosen to limit any unforeseen consequences due to the higher concentration of PAA sanitant.

Table 8 (below) contains the bioburden results from the Protein A affinity chromatographic step of the first engineering run. All samples tested were negative for bioburden as described hereinabove, indicating that the 0.7% (v/v) slurry/packing procedure was effective at eliminating bioburden which allowed for sterile downstream processing for 14 days of continuous downstream processing.

Table 9 (below) contains the bioburden results from the Protein A affinity chromatographic step of the second engineering run. All samples tested were negative for bioburden and the system successfully ran for 14 days of continuous downstream processing. It should be noted however, that these columns may have remained in a sterile state from the previous process.

The 0.7% (v/v) PAA resin slurry and packing procedure followed by a 0.2% (v/v) PAA sanitization that was used in the two engineering runs effectively sanitized the columns and allowed for the continuous Protein A affinity chromatography capture of protein product over a 14 day period. No bioburden was detected in the Protein A step or downstream process for either engineering run (see, Table 8 and Table 9, below), and the BioSMB skid ran as designed over the 14-day period.

TABLE 7
Sanitization Conditions and Results for Column Sanitization Experiments.

| Packing and | | | | Flush | |
|---|---|---|---|---|---|
| Sanitization | | | Sanitization | sample | Bioreactor |
| Procedure | Sanitant | Condition | procedure | testing | recirculation |
| Primary | 0.7% | MabSelect | 5 CV down | No | No |
| | PAA | SuRe resin | 5 CV up | bioburden | bioburden |
| | and | spiked w/ | 1-hour hold | detected | detected |
| | 0.2% | multiple | | | |
| | PAA | organisms; | | | |
| | | slurried, | | | |
| | | packed | | | |
| | | w/0.7% PAA; | | | |
| | | Sanitized with | | | |
| | | 0.2% PAA | | | |
| Alternative 1 | 0.7% | MabSelect | 5 CV down | No | No |
| | PAA | SuRe resin | 5 CV up | bioburden | bioburden |
| | | slurried, | 1-hour hold | detected | detected |
| | | packed and | | | |
| | | sanitized w/ | | | |
| | | 0.7% PAA | | | |
| Alternative 2 | 0.2% | MabSelect | 5 CV down | No | No |
| | PAA/ | SuRe resin | 5 CV up | bioburden | bioburden |
| | 0.1M | spiked w/B. | 1-hour hold | detected | detected |
| | NaCl | subtillis; | | | |
| | | slurried, | | | |
| | | packed w/ | | | |
| | | 0.2% PAA/ | | | |
| | | 0.1M NaCl; | | | |
| | | sanitized with | | | |
| | | 0.2% PAA | | | |
| Alternative 3 | 0.2% | MabSelect | 5 CV down | No | No |
| | PAA | SuRe resin | 5 CV up | bioburden | bioburden |
| | | spiked w/ | 1-hour hold | detected | detected |
| | | multiple | | | |
| | | organisms; | | | |
| | | slurried, | | | |
| | | packed in | | | |
| | | 0.1M NaCl; | | | |
| | | sanitized with | | | |
| | | 0.2% PAA | | | |

TABLE 8
Run 1 Bioburden Results. Samples were pulled from the Protein A
Load, the Protein A Flowthrough, and from the Protein A Eluate.

Production Day

| Sampled Fraction | 8 | 10 | 12 | 15 | 18 | 22 |
|---|---|---|---|---|---|---|
| ProA Load | 0 | 0 | 0 | 0 | 0 | 0 |
| ProA Flowthrough | 0 | 0 | 0 | 0 | 0 | 0 |
| ProA Eluate | ND | ND | 0 | 0 | 0 | 0 |

Samples designated “ND” in Table 8 were not submitted for bioburden analysis. ProA = Protein A affinity chromatography matrix column; ND = no data.

TABLE 9
Run 2 Bioburden Results. Samples were pulled from the Protein A
Load, the Protein A Flowthrough, and from the Protein A Eluate.

Production Day

| Sampled Fraction | 8 | 10 | 12 | 15 | 18 | 22 |
|---|---|---|---|---|---|---|
| ProA Load | 0 | 0 | 0 | 0 | 0 | 0 |
| ProA Flowthrough | 0 | 0 | 0 | 0 | 0 | 0 |
| ProA Eluate | ND | ND | 0 | 0 | 0 | 0 |

Samples designated “ND” in Table 9 were not submitted for bioburden analysis. ProA = Protein A affinity chromatography matrix column; ND = no data.

### Example 4. Continuous Process from Single-Use Bioreactor (SUB) Through Final Tangential Flow Filtration (TFF)

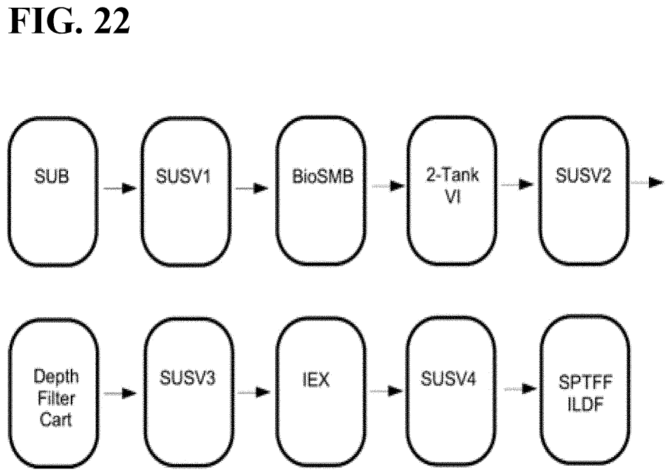

A set of two runs was performed at the 500-L perfusion bioreactor scale to demonstrate a continuous embodiment of the inventive process for manufacturing a purified protein of interest, or a purified protein drug substance—from the single-use perfusion bioreactor to final formulation step to obtain the purified protein drug substance comprising the protein of interest. A flow diagram of the exemplary process is shown in FIG. 22. Downstream of the perfusion bioreactor, the following steps were fluidly connected and operated in a continuous mode: Protein A affinity chromatography capture step on a Cadence™ BioSMB® PD system (Pall), low pH viral inactivation (VI) step on a two-tank Pall Cadence™ Viral Inactivation system, depth filtration step, ion exchange polishing step, and a continuous final formulation step comprised of two-stages of single-pass tangential flow filtration (SPTFF) and in-line diafiltration (ILDF) modules. The viral filter was excluded from these runs, given the additional cost of the filters and the fact that the resulting material was not destined for non-clinical or clinical studies, however in the manufacture of a protein of interest intended for clinical use viral filtration can be included. In between each unit operation, a single-use surge vessel (SUSV) was used to manage flow discrepancies in the process and to react to process upsets. Additionally, the surge vessel before the IEX step was titrated with base to the pH setpoint, and the surge vessel after the IEX step was titrated with acid to the pH setpoint. Results are presented for the overall continuous process performance but focused on the depth filtration and IEX steps as new added steps to the continuous train. The SPTFF-ILDF process and results are discussed in Example 5 hereinbelow.

Materials and Methods. The 500-L single-use perfusion bioreactor was operated in a manner similar to the methods described in Example 1 hereinabove. The downstream process operated continuously for 14 days. The first chromatography system employed was Protein A affinity chromatography, which was performed on the Cadence™ BioSMB® PD system (Pall; “BioSMB”) continuous chromatography system and used four columns. The columns were packed by slurrying the resin in 0.7% (v/v) PAA and packing the resin with 0.7% (v/v) PAA. The packing performance was tested by performing a Height Equivalent Theoretical Plate (HETP) and asymmetry analysis. The packing procedure is a non-sterile step so the columns were sanitized with 0.2% (v/v) PAA after they were attached to the BioSMB immediately prior to starting the process (see, Example 3 herein). The Protein A affinity chromatography step was operated in a manner similar to the methods described in Example 1, including the use of a surge vessel (SUSV1) between the bioreactor and the Protein A system.

Low pH viral inactivation (VI) was performed with a Cadence™ Viral Inactivation system (Pall), which contained two single-use mix tanks for collection, acidification and neutralization. The viral inactivation step was operated in a manner similar to the methods described in Example 2 hereinabove. Multiple Protein A elutions were collected in one of the two VI single-use mix tanks. The Protein A elution pool was adjusted with acid to a low pH and maintained at this pH for a target incubation time to inactivate viruses that might be present. After viral inactivation, the pool was adjusted with base to a neutral pH. This neutralized virally inactivated product pool (NVIP) was then pumped out of the mix tank to SUSV2 which is located prior to the depth filter cart. During acidification, hold, and neutralization, the next series of Protein A elutions were collected in the second VI single-use mix tank of the viral inactivation system. The cycle of alternating collection tanks was repeated for the duration of the Protein A process.

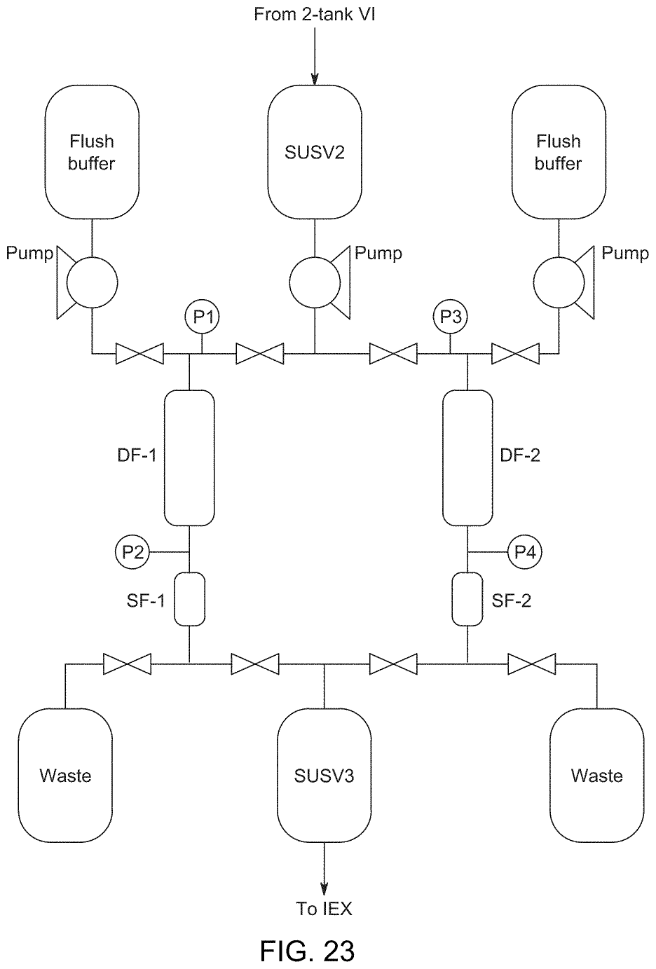

The neutralized virally inactivated product pool (NVIP) was filtered through a depth filter and 0.22 μm filter using a depth filtration cart (see, FIG. 22). A more detailed schematic rendering of the depth filtration cart between the SUSV2 and SUSV3 illustrated in FIG. 22 is found in FIG. 23. Prior to use, the depth filters were autoclaved at 123.1° C. for 60 minutes to reduce any potential bioburden. The depth filters were then installed into filter holders, flushed, sanitized with 0.5N NaOH, and equilibrated with buffer. The filter holders were then installed on the depth filtration cart. Two depth filter/final filter assemblies can be installed on the cart. The NVIP material was filtered through one side (DF-1) until the maximum depth filter throughput was reached. At this point the second depth filter/final filter assembly was put online to receive load material. After reaching the loading target, the first depth filter assembly was flushed with buffer to recover residual product from the depth filter. This occurred in-line and simultaneously with processing with a second pump connected to the depth filter assembly and a buffer bag. Once the flush was completed, a new depth filter assembly was installed. Each set of depth filters was autoclaved and flushed prior to installation in the system. This process is repeated at the throughput limit until the Protein A cycles were complete.

As represented in FIG. 22, the filtered neutralized virally inactivated product pool (FNVIP) was collected in SUSV3 to an appropriate volume prior to loading on to a second chromatography system, which was an ion exchange (IEX) column. The IEX flow-through step was performed on the ÄKTA™ Ready (GE Healthcare Life Sciences) single-use chromatography system. The column and resin were sanitized with 0.5N NaOH prior to use. Prior to the start of the IEX chromatography step, the virally inactivated product pool was pH adjusted by continually adding titrant into SUSV3, as needed. The IEX column effluent absorbance was monitored online at a wavelength of 280 nm and used to collect the IEX pool; this purified product pool comprising the protein was pH adjusted by continually adding titrant into in SUSV4, as needed, for continued processing and was filtered (0.22 μm) to obtain a filtrate prior to the final UF/DF, which would have been a virus-free filtrate in an embodiment including a viral filtration system, e.g., for obtaining clinically usable protein drug substance. (See, FIG. 22). For this continuous IEX step, a single column was used. Since the non-load steps (equilibration, wash, strip, regeneration) require the column to be taken out of load, this resulted in an increase in the SUSV3 volume during the non-load phases of the step. To maintain level control in SUSV3, once the load phase was reinitiated, a higher flow rate than the incoming surge vessel volume flow rate was used to drive down the surge vessel volume. Level control was also maintained in SUSV3 by using automation to vary the pump speed of the IEX chromatography system to maintain a pre-set volume range in the single-use surge vessel.

As described herein, the inventive process leverages SUSVs between continuous unit operations to manage differences in flow, to provide a pressure break between unit operations, and to provide time to react to disturbances in the system. The automation control strategy for these surge vessels was operated as described in Example 1. Two runs were conducted, Run #1 and Run #2, with upstream unit operation conducted in the same manner. For downstream processing, Run #1 was slightly different from Run #2 in that the unit operations of Run #1 were connected and run continuously through the IEX step. In Run #1, the SPTFF/ILDF was not connected to the unit operations upstream, but was run separately in a semi-continuous format. While in Run #2, all the unit operations were connected and run in a continuous format from the perfusion bioreactor through to the SPTFF/ILDF step to obtain the purified protein drug substance comprising the protein of interest. Some control aspects (like level control for SUSV1) were also not working in Run #1, but, otherwise, the process steps and operating parameters were configured in a similar manner.

Results. The perfusion culture process was operated for 22-23 days of production. The continuous downstream process was connected to the 500-L perfusion bioreactor and operated for a total duration of 14 days. Results in Run #1 and Run #2 were similar. As an overall summary of Run #2, there were 39 Protein A cycles per column (total of 156 elution peaks), 53 VI cycles alternating between two tanks, 4 depth filter cycles (with a changeout to a new filter between cycles), and 70 IEX cycles.

All of the upstream and downstream steps were operated as fully/functionally closed systems. A “fully closed” system is defined as a process system that does not expose the product to the room environment, and addition of material to the closed system avoids exposure of the product to the room environment. For example, the upstream bioreactor is a fully closed system, which is never opened to the environment. A “functionally closed” system is defined as a process system that may be opened (e.g., to install a filter or a column) but is rendered back to the closed state by sanitizing the system prior to product introduction, for example, the downstream systems are functionally closed by being rendered closed through the use of a sanitant. (Palberg et al., Challenging the Cleanroom Paradigm for Biopharmaceutical Manufacturing of Bulk Drug, Substances, BioPharm International Volume 24, Issue 8 (2011)). All of the systems were set up with gamma-irradiated and autoclaved components, aseptic connectors or weldable tubing for connections, and a single-use air break assembly for waste lines to establish a closed system boundary for each step. The systems were rendered functionally closed by sanitizing the components that could not be gamma-irradiated or autoclaved. As described in the methods section in this Example 4, the Protein A columns and resin were sanitized with peracetic acid (PAA), and the depth filter membrane, IEX column and resin, and SPTFF-ILDF membranes were sanitized with 0.5N NaOH. Bioburden and endotoxin data, sampled from multiple points in the process on different days are summarized below in Table 10 and Table 11. The results demonstrate that using the proper procedures to operate a continuous process as a fully/functionally closed system can successfully achieve a state of low bioburden control.

TABLE 10
Summary of bioburden results (CFU/10 mL) for 500-L Run #2 in
the embodiment schematically illustrated in FIG. 22.

| | Production Day | | | | | |
|---|---|---|---|---|---|---|
| Step (Sampling Point) | 8 | 10 | 12 | 15/16 | 19 | 22/23 |
| Bioreactor | 0 | | 0 | 0 | 0 | 0 |
| ProA Load (SUSV1) | 0 | 0 | 0 | 0 | 0 | 0 |
| ProA Flowthrough | 0 | 0 | 0 | 0 | 0 | 0 |
| ProA Elution (VI Mixer) | | | 0 | 0 | 0 | 0 |
| Neutralized VI Pool (SUSV2) | | 0 | 0 | 0 | 0 | 0 |
| IEX Load (SUSV3) | | 0 | 0 | 0 | 0 | 0 |
| IEX Pool (SUSV4) | | 0 | 0 | 0 | 0 | 0 |
| ILDF Retentate/Final Pool | | | 0 | 0 | 0 | 0 |

ProA = Protein A affinity chromatography matrix column.

TABLE 11
Summary of endotoxin results (EU/mL) for 500-L Run #2 in
embodiment schematically illustrated in FIG. 22.

| | Production Day | | | | |
|---|---|---|---|---|---|
| Step (Sampling Point) | 8 | 12 | 15/16 | 19 | 22/23 |
| Bioreactor | <3 | <3 | | <3 | <3 |
| ProA Load (SUSV1) | <0.6 | <0.6 | <0.6 | <0.6 | <0.6 |
| ProA Flowthrough | <0.6 | <0.6 | <0.6 | <0.6 | <0.6 |
| ProA Elution (VI Mixer) | | | <0.6 | <0.6 | <0.6 |
| Neutralized VI Pool (SUSV2) | | <0.6 | <0.6 | <0.6 | <0.6 |
| IEX Load (SUSV3) | | <0.6 | <0.6 | <0.6 | <0.6 |
| IEX Pool (SUSV4) | | <0.6 | <0.6 | <0.6 | <0.6 |
| ILDF Retentate/Final | | <0.6 | <0.6 | <0.6 | <0.6 |
| Pool | | | | | |

ProA = Protein A affinity chromatography matrix column.

### Example 5. Single Pass Tangential Flow Filtration/In-Line Diafiltration (SPTFF/ILDF)

In this example a set of two runs of the inventive process described in Example 4 was performed in a fully continuous mode from the bioreactor to the final formulation step, including single pass tangential flow filtration (SPTFF) and in-line diafiltration (ILDF). The benefits of SPTFF and ILDF include minimizing the need for large in-process holding vessels or tanks, as well reducing the time a product pool need be held in a potentially less stable condition than the final formulation condition. Also, potentially less filter area can be used than in a traditional batch process. There were challenges to implementation of a continuous process format, however, which included: long duration operation for both the SPTFF and ILDF devices and previously unknown fouling characteristics over long durations; the previously unknown impact of varying incoming feed flow rates due to adjustments made by automation to maintain surge tank volumes, as well as matching flow rates between different unit operations; and maintaining clean processing over long durations. As described further hereinbelow, the present invention met all these challenges.

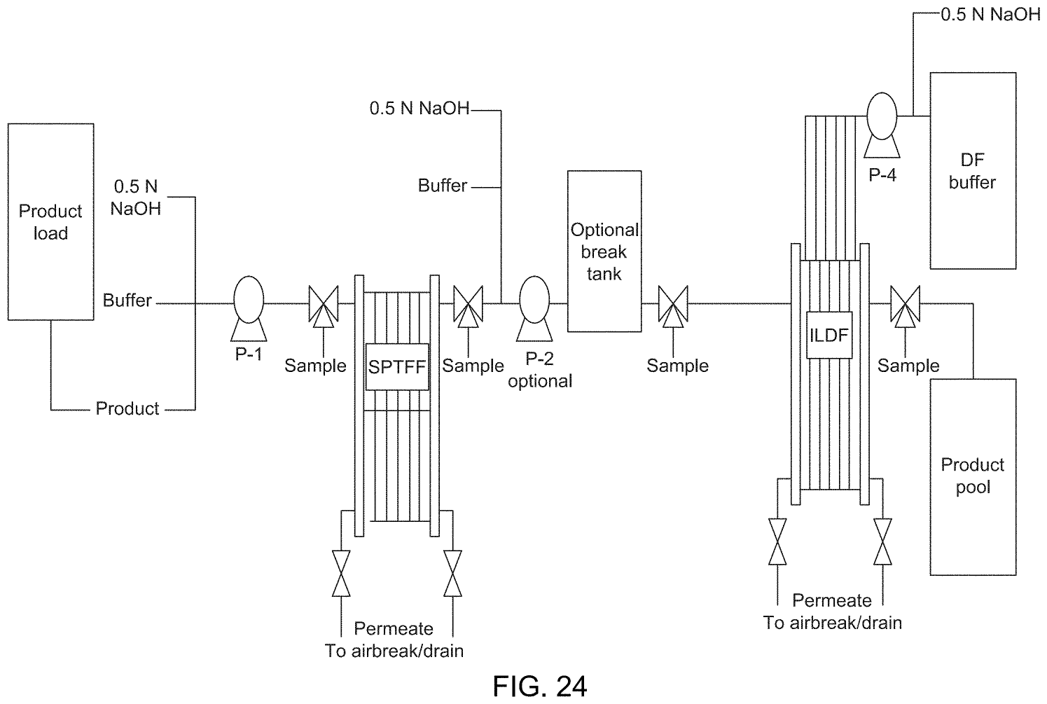

Methods and Materials. The preceding manufacturing process steps and sanitization prior to the SPTFF/ILDF step were described in Example 3 and Example 4 hereinabove. For the downstream single pass tangential flow filtration (SPTFF) and inline diafiltration unit operations, a Cadence™ Single Pass TFF Module (SPTFF; Pall) and a Cadence™ Inline Diafiltration (ILDF; Pall) device were used for both runs. Both devices were multi-staged tangential flow filtration (TFF) cassettes, and new membranes were used for each run. The SPTFF and ILDF devices and the flow path are illustrated schematically in FIG. 24. For the setup in Run #1, all of the tubing assemblies were autoclaved for the SPTFF portion of the system, whereas for the ILDF portion of the system, some sections of the system were only sanitized with 0.5N NaOH and not autoclaved; this resulted in observed bioburden in the ILDF pool. In order to mitigate this issue for Run #2, all lines, flow meters and pressure sensors were autoclaved, except conductivity sensors which were not autoclavable. The conductivity sensors were sprayed with sanitant (Minncare®) and attached to the lines in a sterile hood. After attaching the process lines to the SPTFF and ILDF devices, the membranes and flowpath were sanitized prior to use with 0.5N NaOH. Pre-use flushing was performed at faster flow rates while keeping pressures <20 psi. Post-sanitization, the system was operated as functionally closed, with all connections being made by weldable tubing or commercially obtained AseptiQuik® connections (Colder Products Company). The feed flow rate for the SPTFF (from SUSV4) matched the product effluent flow rate from the preceding column step. A retentate pump controlled the retentate flow rate, thus controlling the product concentration factor in the SPTFF device. For the SPTFF step, product is concentrated in the filter. A set retentate flow rate (e.g., 5-10 times lower than the feed flow rate depending on the desired concentration factor) determines the amount of buffer removed through the permeate, and concentrates the retained product. The ILDF feed was connected to the retentate of the SPTFF with an optional break tank, i.e., a surge vessel, between the steps. In this example, in Run #1 an autoclaved 2-L bioreactor with mixer served as the optional surge vessel between the SPTFF and ILDF steps, however another type of surge vessel, e.g., single-use surge vessel (SUSV), can also be employed. The schematic diagram of the SPTFF/ILDF set-up (FIG. 24) shows the Run #1 set-up in which an autoclaved 2-L surge vessel with mixer (designated “Break Tank” in FIG. 24) between the SPTFF and the ILDF was used for the majority of the run. The surge vessel was removed for the last few days of the run as shown in the data below. In Run #2, no surge vessel was placed between SPTFF and ILDF unit operations. Run #2 had the SPTFF retentate line connected directly to the ILDF feed line. This connection not only eliminated the need for the surge vessel, but it also eliminated pump 2 from the set-up. The feed flow rate of the ILDF matched the retentate flow rate of the SPTFF. The retentate of the ILDF was controlled at the same rate as the feed flow. A separate diafiltration pump fed formulation buffer into multiple channels of the ILDF device. Conversion to formulation buffer was controlled by the ratio of the diafiltration feed flow to the ILDF feed/retentate flows. The permeates for both devices were sent to drain through an air break as described in previous examples.

Results. The SPTFF and ILDF modules performed consistently in fully continuous mode over the duration of both runs, with no apparent signs of fouling or reduced performance over 12 days, as evidenced by a consistent pressure of less than 5 psi for the SPTFF and consistent pressure less than 15 psi for the ILDF throughout the run duration. Both systems recovered quickly after interruptions that stopped all pumps. The concentration factor of the product was maintained throughout the run duration, at a predetermined value of about 5-10 times lower than the feed flow rate, based on the desired concentration factor, and the final conductivity of the pool was matched to the starting diafiltration buffer. A total of 4.8 kg mass output of product was processed for Run 1 and 7.3 kg mass output of product was processed for Run #2. Overall yield for Run 1 was 98%; overall yield for Run 2 was not calculated.

The foregoing are merely exemplary, and the skilled practitioner of the present invention can easily vary the components and operating parameters as needed for a particular recombinant therapeutic protein drug substance of interest.

1. A process for manufacturing a purified protein of interest, the process comprising the step of:
(a) culturing mammalian cells in one or more single-use perfusion bioreactors comprising a liquid culture medium under conditions that allow the cells to secrete the protein into the liquid culture medium for a production cultivation period of at least 10 days, wherein, periodically or continuously, during the production cultivation period, fresh sterile liquid culture medium is added into the one or more perfusion bioreactors, being mixed contemporaneously from a plurality of different concentrated medium component solutions and an aqueous diluent, to maintain a constant culture volume in each of the perfusion bioreactor(s), in direct relation to volumes of the culture that are continuously or periodically removed from each of the perfusion bioreactor(s) as volumes of permeate or cell bleed, and wherein the removed volumes of permeate are automatically and fluidly fed from the one or more single-use perfusion bioreactor(s) into a single-use surge vessel and thence into a first chromatography system, whereby the protein is collected in a protein isolate fraction.

2. The process of claim 1, further comprising the step of:
(b) switching the protein isolate fraction into a low pH or detergent viral inactivation system and, if needed, a neutralization system, to obtain a virally inactivated product pool comprising the protein.

3. The process of claim 2, further comprising the steps of:
(c) introducing the virally inactivated product pool into a second chromatography system to obtain a purified product pool comprising the protein;
(d) switching the purified product pool comprising the protein into an optional third chromatography system and/or a viral filtration system to obtain a virus-free filtrate comprising the protein; and
(e) switching the virus-free filtrate into an ultrafiltration/diafiltration system to obtain a composition comprising the purified protein of interest.

4. The process of claim 1, wherein the protein of interest is a recombinant protein.

5. The process of claim 1, wherein the protein of interest is a therapeutic protein.

6. The process of claim 1, claim 2, or claim 3, wherein one or more of the first chromatography system, the second chromatography system, the third chromatography system, the low pH or detergent viral inactivation system, the neutralization system, the viral filtration system, or the ultrafiltration/diafiltration system, comprise a single-use component(s).

7. The process of claim 1, wherein the mammalian cells are cultured in two, three, four, five, or six single-use perfusion bioreactors.

8. The process of claim 1, wherein the one or more single-use bioreactor(s) can contain a volume of liquid culture medium about 50 L to about 4000 L.

9. The process of claim 1, wherein the fresh sterile liquid culture medium is added to the one or more perfusion bioreactors, by injecting the plurality of different concentrated medium component solutions at fixed ratios relative to one another, directly into the perfusion bioreactor(s), while an aqueous diluent is also added at varied ratio(s) relative to the plurality of different concentrated component solutions, to maintain a constant culture volume in each perfusion bioreactor(s).

10. The process of claim 1, wherein the fresh sterile liquid culture medium is added to the one or more perfusion bioreactors, by injecting the plurality of different concentrated medium component solutions and the aqueous diluent at fixed ratios relative to one another, directly into the perfusion bioreactor(s), to maintain a constant culture volume in each perfusion bioreactor(s).

11. The process of claim 1, wherein the fresh sterile liquid culture medium is added to the one or more perfusion bioreactors, by injecting the plurality of different concentrated medium component solutions and the aqueous diluent, at fixed ratios relative to one another, into a mixing chamber wherein fresh sterile liquid culture medium is mixed contemporaneously before being added to each perfusion bioreactor(s) to maintain a constant culture volume.

12. The process of claim 1, wherein an automated controller comprising a detector is used to measure the fluid volume in the single-use surge vessel, and a processor varies the pump speeds of the first chromatography system to maintain a pre-set volume range in the single-use surge vessel.

13. The process of claim 2 or claim 3, wherein one or more of steps (b), (c), (d), or (e) is performed automatically and fluidly in an uninterrupted flow from the previous step, and wherein a surge vessel is employed between one or more steps, and a processor varies the pump speed in a subsequent step to regulate the pre-set volume range of the surge vessel preceding the subsequent step.

14. The process of claim 13, wherein in-line or in-vessel conditioning of pH and/or conductivity load is performed between the one or more of steps (b), (c), (d), or (e).

15. The process of claim 1, wherein:
(i) a process automation system is in electronic communication with at least the one or more single-use perfusion bioreactors, the single-use surge vessel, and the first chromatography system;
(ii) the process automation system stores a first set of control modules to control operation of at least one single-use perfusion bioreactor of the one or more single-use perfusion bioreactors;
(iii) the process automation system stores a second set of control modules to control operation of feed tanks;
(iv) the process automation system stores a third set of control modules to control operation of collection tanks; and
(v) the at least one single-use perfusion bioreactor is logically configured to be coupled to one or more feed tanks, one or more collection tanks, or a filter bank.

16. The process of claim 15, wherein the at least one single-use perfusion bioreactor is disposed on a skid and the skid includes a plurality of communication interfaces to electronically couple the at least one single-use perfusion bioreactor to a plurality of pieces of portable equipment.

17. The process of claim 16, further comprising:
determining, by the process automation system, that the single-use surge vessel has been coupled to a communication interface of the plurality of communication interfaces based on data received via the communication interface, the data indicating an identifier of the single-use surge vessel and a function of the single-use surge vessel; and
determining, based at least partly on the identifier and the function of the single-use surge vessel, that the single-use surge vessel is a collection tank and that the third set of control modules is to control operation of the single-use surge vessel.

18. The process of claim 17, further comprising:
determining, by the process automation system, that a mixing vessel has been coupled to an additional communication interface of the plurality of communication interfaces based on additional data received via the additional communication interface, the additional data indicating an additional identifier of the mixing vessel and an additional function of the mixing vessel; and
determining, based at least partly on the additional identifier and the additional function of the mixing vessel, that the mixing vessel is a feed tank and that the second set of control modules is to control operation of the mixing vessel.

19. The process of claim 1, wherein the production cultivation period is at least 20 days.

20-47. (canceled)

48. The process of claim 1, wherein the process is conducted in a continuous format.

49. The process of claim 1, wherein the first chromatography system is sanitized with a chemical sanitant solution comprising peracetic acid before use.

50. The process of claim 3, wherein the ultrafiltration/diafiltration system comprises a single pass tangential flow filtration (SPTFF), and the operating pressure of the SPTFF is controlled in a range of about 0.25 psi to about 60 psi.

51. The process of claim 3, wherein the ultrafiltration/diafiltration system comprises inline depth filtration (ILDF), and the operating pressure of the ILDF is controlled in a range of about 0.25 psi to about 60 psi.

52. The process of claim 2, wherein the process is conducted in a continuous format.

53. The process of claim 3, wherein the process is conducted in a continuous format.

54. The process of claim 6, wherein the process is conducted in a continuous format.

55. The process of claim 2, wherein the first chromatography system is sanitized with a chemical sanitant solution comprising peracetic acid before use.

56. The process of claim 3, wherein the first chromatography system is sanitized with a chemical sanitant solution comprising peracetic acid before use.

57. The process of claim 6, wherein the first chromatography system is sanitized with a chemical sanitant solution comprising peracetic acid before use.
